SEQUENCE LISTING (6 sequences)

SEQ ID NO: 1 - DNA, 20 bp, synthetic construct
  misc_feature 1..20: note = Description of Artificial Sequence: Synthetic oligonucleotide
  source 1..20: mol_type = other DNA
tgctgtgtgtttgctcaaac

SEQ ID NO: 2 - DNA, 20 bp, synthetic construct
  misc_feature 1..20: note = Description of Artificial Sequence: Synthetic oligonucleotide
  source 1..20: mol_type = other DNA
atctccagcaaagtgaaacc

SEQ ID NO: 3 - DNA, 20 bp, synthetic construct
  misc_feature 1..20: note = Description of Artificial Sequence: Synthetic oligonucleotide
  source 1..20: mol_type = other DNA
gggcgaggagctgttcaccg

SEQ ID NO: 4 - DNA, 58 bp, synthetic construct
  misc_feature 1..58: note = Description of Artificial Sequence: Synthetic oligonucleotide
  source 1..58: mol_type = other DNA
ccgggctcaccaagacttacacattctcgagaatgtgtaagtcttggtgagctttttg

SEQ ID NO: 5 - DNA, 58 bp, synthetic construct
  misc_feature 1..58: note = Description of Artificial Sequence: Synthetic oligonucleotide
  source 1..58: mol_type = other DNA
ccggcctgggattgatggagttaaactcgagtttaactccatcaatcccaggtttttg

SEQ ID NO: 6 - DNA, 57 bp, synthetic construct
  misc_feature 1..57: note = Description of Artificial Sequence: Synthetic oligonucleotide
  source 1..57: mol_type = other DNA
ccgggcgcgatagcgctaataatttctcgagaaattattagcgctatcgcgcttttt

(19) United States
(12) Patent Application Publication
(10) Pub. No.: US 2026/0338763 A1
(43) Pub. Date: Oct. 29, 2026

(54) METHODS AND KITS FOR PREDICTING CANCER PROGNOSIS AND METASTASIS

(71) Applicant: The Regents of the University of California, Oakland, CA (US)

(72) Inventors: Stephanie Fraley, La Jolla, CA (US); Hannah Carter, La Jolla, CA (US); Daniel Ortiz Velez, La Jolla, CA (US); Brian Tsui, La Jolla, CA (US)

(21) Appl. No.: 19/202,401

(22) Filed: May 8, 2025

Related U.S. Application Data
(63) Continuation of application No. 18/110,341, filed on Feb. 15, 2023, now abandoned.
Application No. 18/110,341 is a continuation of application No. 16/465,991, filed on Dec. 1, 2017, now abandoned.
(60) Provisional application No. 62/429,581, filed on Dec. 2, 2016.

(51) Int. Cl. G01N 33/50; C12N 5/09; C12Q 1/6886; G01N 33/575

(52) U.S. Cl. CPC G01N 33/5011; C12N 5/0693; C12Q 1/6886; G01N 33/5023; G01N 33/5758; C12N 2533/54; C12N 2533/90; C12Q 2600/118; C12Q 2600/158; G01N 2800/52

(57) ABSTRACT

Disclosed herein is a novel gene signature of metastatic cancer cells and a novel three-dimensional (3D) culture system for use in improved methods of predicting metastasis or prognosis in cancer. Accordingly, described herein are methods of determining a risk of metastasis, methods of predicting prognosis for cancer patients, methods of treating a cancer patient identified at high risk of metastasis, methods of treating a cancer patient identified as having poorer prognosis, methods for determining the migration capacity of a tumor, methods of screening a tumor for sensitivity to a drug, and kits for use in performing these methods.

### CROSS-REFERENCE TO RELATED APPLICATION

This application is a continuation of U.S. Ser. No. 18/110,341, filed Feb. 15, 2023, which is a continuation of U.S. Ser. No. 16/465,991, filed May 31, 2019, which is a national stage application under 35 U.S.C. § 371 of International Application No. PCT/US2017/064348, filed Dec. 1, 2017, which claims the benefit under 35 U.S.C. 119 (e) of U.S. Provisional Ser. No. 62/429,581, filed Dec. 2, 2016, the contents of each of which are hereby incorporated by reference in their entireties.

### SEQUENCE LISTING

The instant application contains a Sequence Listing which has been submitted electronically in XML format and is hereby incorporated by reference in its entirety. Said XML copy, created on Jul. 12, 2023, is named 114198-0921.xml and is 8.85 kilobytes in size.

### TECHNICAL FIELD

The methods, kits, and systems disclosed herein relate to the field of gene expression and phenotypic analysis for determining the risk of metastasis or poorer prognosis for a cancer patient.

### BACKGROUND

The following discussion of the background of the invention is merely provided to aid the reader in understanding the invention and is not admitted to describe or constitute prior art to the present invention.

An initial step in cancer metastasis is the migration of tumor cells through the extracellular matrix (ECM) and into the lymphatic or vascular systems. Several features of the tumor ECM have been associated with progression to metastasis. In particular, regions of dense collagen are co-localized with aggressive tumor cell phenotypes in numerous solid tumors, including breast, ovarian, pancreatic, and brain cancers. However, sparse and aligned collagen fibers at the edges of tumors have also been reported to correlate with aggressive disease. Cancer cells migrating through densely packed collagen within the tumor use invadopodia and matrix metalloproteinase (MMP) activity to move, whereas cells in regions with less dense collagen with long, aligned fibers migrate rapidly using larger pseudopodial protrusions or MMP-independent amoeboid blebbing. It remains unclear whether and how these local migration behaviors contribute to the formation of distant metastases and whether collagen architecture functionally contributes to metastatic migration or is only a correlative hallmark of the process.

This question is further complicated by significant heterogeneity in the intrinsic ability of tumor cells to migrate and metastasize. Current methods of predicting metastasis or prognosis in cancer (e.g., MammaPrint and Oncotype DX) leave a great deal to be desired in terms of predictive power. Thus, there is a need in the art of better predicting metastasis and survival in cancer patients. The present disclosure satisfies this need.

### SUMMARY OF THE DISCLOSURE

Described herein is a novel gene signature of metastatic cancer cells and a novel three-dimensional (3D) culture system for use in improved methods of predicting metastasis or prognosis in cancer. Accordingly, described herein are novel methods of determining a risk of metastasis, novel methods of predicting prognosis for cancer patients, novel methods of treating a cancer patient identified at high risk of metastasis, novel methods of treating a cancer patient identified as having poorer prognosis, novel methods for determining the migration capacity of a tumor, novel methods of screening a tumor for sensitivity to a drug, and novel kits and culture systems for use in performing these methods.

Accordingly, in one aspect provided herein is a method of determining gene expression level of one or more genes of a vascular mimicry (VM) gene module in a sample isolated from a subject, comprising, or consisting of, or consisting essentially of, analyzing the expression of the one or more genes listed in the VM gene module. In some embodiments, the method further comprises, or alternatively consisting essentially of, or yet further consisting of, determining a risk of tumor metastasis in the subject by comparing a change in expression of the one or more genes in the VM gene module compared to a predetermined reference level. In one aspect, the predetermined reference level is the gene expression of level in a normal, non-diseased counterpart tissue.

In some embodiments, the one or more genes of the VM gene module comprise, consist of, or consist essentially of genes selected from COL5A1, FRMD6, TANC2, THBS1, PEAK1, ITGAV, DAAM1, RASEF, JAG1, LAMC2, ZNF532, SKIL, NAV1, ARHGAP32, SYNE1, GALNT10, LHFPL2, ABL2, LTBP1, COL4A1, DPY19L1, LPCAT2, TBC1D2B, LAMB1, AMIGO2, NREP, SNX30, TPM1, COL4A2, ARNTL, MRC2, TGFBI, TVP23C, BHLHE40, SMAD7, ABLIM3, ZNF224, PODXL, TAGLN, VHL, EPHB2, EDN1, GTF2IP4, HPS4, SIPA1L1, PID1, NLGN2, LTBP4, TRMT13, IGF2BP3, RBPJ, MKL1, ZMYM5, EFCAB11, WDR66, NKX3-1, HMOX1, TYRO3, SDHAP1, FURIN, FAM43A, AGTRAP, KCTD11, ID2, FERMT1, MTND2P28, H2BFS, LFNG, HES1, or KIN, or an equivalent of each thereof.

In some embodiments, the VM gene module comprises, consists of, or consists essentially of at least one, at least two, at least three, or four genes selected from ITGB1, LAMC2, COL4A1, and DAAM1, or an equivalent of each thereof.

In some embodiments, the gene expression level is determined by a method comprising, or consisting essentially of, or yet consisting of, determining the amount of an mRNA transcribed from the one or more genes of the VM gene module. In some embodiments, the gene expression level is determined by a method comprising, consisting of, or consisting essentially of one or more of in situ hybridization, northern blot, PCR, quantitative PCR, RNA-seq, or microarray. In some embodiments, the change in expression of the genes in the VM gene module is increased as compared to the predetermined reference level. In one aspect, the predetermined reference level is the gene expression of level in a normal, non-diseased counterpart tissue.

In some embodiments, the sample is a tumor sample. In some embodiments, the tumor sample is at least one of a fixed tissue, a frozen tissue, a biopsy tissue, a circulating tumor cell liquid biopsy, a resection tissue, a microdissected tissue, or a combination thereof. In particular embodiments, the sample is a biopsy tissue sample or a circulating tumor cell liquid biopsy sample.

In some embodiments, the subject has been diagnosed with cancer. In some embodiments, the cancer is a stage I or stage II cancer. In some embodiments, the cancer is selected from breast cancer, glioma, cervical squamous cell carcinoma, endocervical adenocarcinoma, lung adenocarcinoma, kidney renal clear cell carcinoma, and pancreatic adenocarcinoma.

In some embodiments, the method further comprises, or consisting essentially of, or yet consisting of, the step of culturing the sample in a high density 3D collagen culture system and determining the sample's migration capacity. In some embodiments, the method further comprises, or alternatively consisting essentially of, or yet further consists of, administering a cancer treatment comprising, or alternatively consisting essentially of, or yet further consisting of chemotherapy, that is optionally an aggressive treatment, and/or radiation therapy.

In some embodiments, the subject is a mammal. In some embodiments, the subject is an equine, bovine, canine, feline, murine, or a human. In a particular embodiment, the subject is a human.

In another aspect, disclosed herein is a method of predicting prognosis for a cancer patient, the method comprising, consisting of, or consisting essentially of, determining a gene expression level of one or more genes of a vascular mimicry (VM) gene module in a sample isolated from the cancer subject, wherein an increase in expression of the one or more genes in the VM gene module compared to a predetermined reference level is indicative of poor prognosis. In one aspect, increased expression intends an expression level of the gene over and above the expression of the gene in a counterpart, normal tissue not having a phenotype of the disease.

In some embodiments, the one or more genes of the VM gene module comprise, consist of, or consist essentially of genes selected from COL5A1, FRMD6, TANC2, THBS1, PEAK1, ITGAV, DAAM1, RASEF, JAG1, LAMC2, ZNF532, SKIL, NAV1, ARHGAP32, SYNE1, GALNT10, LHFPL2, ABL2, LTBP1, COL4A1, DPY19L1, LPCAT2, TBC1D2B, LAMB1, AMIGO2, NREP, SNX30, TPM1, COL4A2, ARNTL, MRC2, TGFBI, TVP23C, BHLHE40, SMAD7, ABLIM3, ZNF224, PODXL, TAGLN, VHL, EPHB2, EDN1, GTF2IP4, HPS4, SIPA1L1, PID1, NLGN2, LTBP4, TRMT13, IGF2BP3, RBPJ, MKL1, ZMYM5, EFCAB11, WDR66, NKX3-1, HMOX1, TYRO3, SDHAP1, FURIN, FAM43A, AGTRAP, KCTD11, ID2, FERMT1, MTND2P28, H2BFS, LFNG, HES1, or KIN, and equivalents of each thereof.

In some embodiments, the VM gene module comprises, consists of, or consists essentially of at least one, at least two, at least three, or four genes selected from ITGB1, LAMC2, COL4A1, and DAAM1, and equivalents of each thereof.

In some embodiments, the gene expression level is determined by a method comprising determining the amount of an mRNA transcribed from the one or more genes of the VM gene module. In some embodiments, the gene expression level is determined by a method comprising, consisting of, or consisting essentially of one or more of in situ hybridization, northern blot, PCR, quantitative PCR, RNA-seq, or microarray. In some embodiments, the change in expression of the genes in the VM gene module is increased compared to the predetermined reference level. In one aspect, the predetermined reference level is the gene expression of level in a normal, non-diseased counterpart tissue.

In some embodiments, the sample is a tumor sample. In some embodiments, the tumor sample is at least one of a fixed tissue, a frozen tissue, a biopsy tissue, a circulating tumor cell liquid biopsy, a resection tissue, a microdissected tissue, or a combination thereof. In particular embodiments, the sample is a biopsy tissue sample or a circulating tumor cell liquid biopsy sample.

In some embodiments, the subject has been diagnosed with cancer. In some embodiments, the cancer is a stage I or stage II cancer. In some embodiments, the cancer is selected from breast cancer, glioma, cervical squamous cell carcinoma, endocervical adenocarcinoma, lung adenocarcinoma, kidney renal clear cell carcinoma, and pancreatic adenocarcinoma.

In some embodiments, the method further comprises the step of culturing the sample in a high density 3D collagen culture system and determining the sample's migration capacity. In some embodiments, cell migration is indicative of poorer or poor prognosis. In some embodiments, the method further comprises, or alternatively consists essentially of, or yet further consists of administering to the subject a cancer treatment comprising chemotherapy, that is optionally an aggressive treatment, and/or radiation therapy.

In some embodiments, the subject is a mammal. In some embodiments, the subject is an equine, bovine, canine, feline, murine, or a human. In a particular embodiment, the subject is a human.

In another aspect, provided herein is a method of treating a cancer patient, the method comprising, consisting of, or consisting essentially of administering a cancer treatment that is optionally an aggressive cancer treatment to the cancer patient, wherein a sample isolated from the cancer patient has previously been determined to have increased expression of one or more VM module genes compared to a predetermined reference level. In one aspect, increased expression intends an expression level of the gene over and above the expression of the gene in a counterpart, normal tissue not having a phenotype of the disease.

In some embodiments, the one or more genes of the VM gene module comprise, consist of, or consist essentially of genes selected from COL5A1, FRMD6, TANC2, THBS1, PEAK1, ITGAV, DAAM1, RASEF, JAG1, LAMC2, ZNF532, SKIL, NAV1, ARHGAP32, SYNE1, GALNT10, LHFPL2, ABL2, LTBP1, COL4A1, DPY19L1, LPCAT2, TBC1D2B, LAMB1, AMIGO2, NREP, SNX30, TPM1, COL4A2, ARNTL, MRC2, TGFBI, TVP23C, BHLHE40, SMAD7, ABLIM3, ZNF224, PODXL, TAGLN, VHL, EPHB2, EDN1, GTF2IP4, HPS4, SIPA1L1, PID1, NLGN2, LTBP4, TRMT13, IGF2BP3, RBPJ, MKL1, ZMYM5, EFCAB11, WDR66, NKX3-1, HMOX1, TYRO3, SDHAP1, FURIN, FAM43A, AGTRAP, KCTD11, ID2, FERMT1, MTND2P28, H2BFS, LFNG, HES1, or KIN, and equivalents of each thereof.

In some embodiments, the VM gene module comprises, consists of, or consists essentially of at least one, at least two, at least three, or four genes selected from ITGB1, LAMC2, COL4A1, and DAAM1, and equivalents of each thereof.

In some embodiments, the gene expression level is determined by a method comprising determining the amount of an mRNA transcribed from the one or more genes of the VM gene module. In some embodiments, the gene expression level is determined by a method comprising, consisting of, or consisting essentially of one or more of in situ hybridization, northern blot, PCR, quantitative PCR, RNA-seq, or microarray. In some embodiments, the change in expression of the genes in the VM gene module is increased as compared to expression of the gene in a counterpart, normal tissue not having a phenotype of the disease.

In some embodiments, the sample is a tumor sample. In some embodiments, the tumor sample is at least one of a fixed tissue, a frozen tissue, a biopsy tissue, a circulating tumor cell liquid biopsy, a resection tissue, a microdissected tissue, or a combination thereof. In particular embodiments, the sample is a biopsy tissue sample or a circulating tumor cell liquid biopsy sample.

In some embodiments, the cancer is a stage I or stage II cancer. In some embodiments, the cancer is selected from breast cancer, glioma, cervical squamous cell carcinoma, endocervical adenocarcinoma, lung adenocarcinoma, kidney renal clear cell carcinoma, and pancreatic adenocarcinoma.

In some embodiments, the method further comprises the step of culturing the sample in a high density 3D collagen culture system and determining the sample's migration capacity. In some embodiments, cell migration as compared to a normal counterpart cell, is indicative of poorer or poor prognosis.

In some embodiments, the subject is a mammal. In some embodiments, the cancer patient is an equine, bovine, canine, feline, murine, or a human. In a particular embodiment, the cancer patient is a human.

In some embodiments, the sample has previously been determined to migrate in a high density 3D collagen culture system.

In some embodiments, the cancer treatment and optional aggressive cancer treatment comprises chemotherapy and/or radiation therapy.

In another aspect, provided herein is a kit for determining the gene expression level and/or a risk of tumor metastasis, the kit comprising, consisting of, or consisting essentially of reagents for determining the gene expression level of at least one VM module gene in a sample isolated from a subject, and instructions for use.

In another aspect, provided herein is a method of determining the migration capacity of a tumor comprising tumor cells, the method comprising, consisting of, or consisting essentially of, culturing a tumor sample embedded in a 3D collagen matrix, wherein the tumor sample was isolated from a subject; and determining the migration capacity of the tumor sample by tracking motility of the tumor cells in the 3D collagen matrix.

In some embodiments, the 3D collagen matrix comprises a high density of collagen. In some embodiments, the collagen density is selected from the group of, from about 4 mg/mL to about 10 mg/mL, from about 4 mg/mL to about 8 mg/mL, or from about 4 mg/mL to about 6 mg/mL. In a particular embodiment, the collagen density is about 6 mg/mL.

In some embodiments, the 3D collagen matrix comprises, consists of, or consists essentially of a median fiber length less than or equal to 9.5 μm. In some embodiments, the 3D collagen matrix comprises, consists of, or consists essentially of a median pore size less than or equal to 10 μm.

In some embodiments, the 3D collagen matrix further comprises a molecular crowding agent. In some aspects it is selected from polyethylene glycol (PEG), polyvinyl alcohol, dextran and ficoll. In a particular embodiment, the molecular crowding agent is selected from polyethylene glycol (PEG), polyvinyl alcohol, dextran and ficoll. In one aspect it is PEG.

In some embodiments, motility is tracked by imaging the embedded tumor sample. In some embodiments, the embedded tumor sample is imaged at least once per day. In other embodiments, the embedded tumor sample is imaged at least once every two days. In other embodiments, the embedded tumor sample is imaged at least once every three days. In some embodiments, at least one image of the embedded tumor sample is analyzed to characterize tumor cell migration and/or motility. In some embodiments, the image is analyzed using an image processing algorithm.

In some embodiments, the method further comprises determining an invasion distance of a tumor cell, quantifying network structures formed by the tumor cells, determining the length of network structures formed by the tumor cells, and or/determining the shape of a tumor cell. These can be noted as the staging of the tumor and/or tumor cells, as known to those of skill in the art.

In particular embodiments, the 3D collagen matrix comprises, consists of, or consists essentially of about 2 mg/mL to about 6 mg/mL collagen and at least 4 mg/mL PEG.

In some embodiments, the method further comprises determining a gene expression level of one or more genes of a VM gene module in the tumor sample.

In some embodiments, the tumor sample is a biopsy tissue sample or a circulating tumor cell liquid biopsy sample.

In another aspect, provided herein is a method of screening a tumor for sensitivity to a drug, the method comprising, consisting of, or consisting essentially of, culturing a tumor sample embedded in a 3D collagen matrix comprising one or more drugs; and screening the tumor sample for sensitivity to the drug by determining the viability of the tumor sample.

In some embodiments, the 3D collagen matrix comprises a high density of collagen. In some embodiments, the collagen density is selected from the group of, from about 4 mg/mL to about 10 mg/mL, from about 4 mg/mL to about 8 mg/mL, or from about 4 mg/mL to about 6 mg/mL. In a particular embodiment, the collagen density is about 6 mg/mL.

In some embodiments, the 3D collagen matrix comprises, consists of, or consists essentially of a median fiber length less than or equal to 9.5 μm. In some embodiments, the 3D collagen matrix comprises, consists of, or consists essentially of a median pore size less than or equal to 10 μm.

In some embodiments, the 3D collagen matrix further comprises a molecular crowding agent selected from polyethylene glycol (PEG), polyvinyl alcohol, dextran and ficoll. In a particular embodiment, the molecular crowding agent is polyethylene glycol (PEG).

In some embodiments, the method further comprising tracking the motility of the tumor sample. In some embodiments, motility is tracked by imaging the embedded tumor sample. In some embodiments, the embedded tumor sample is imaged at least once per day. In other embodiments, the embedded tumor sample is imaged at least once every two days. In other embodiments, the embedded tumor sample is imaged at least once every three days. In some embodiments, at least one image of the embedded tumor sample is analyzed to characterize tumor cell migration and/or motility. In some embodiments, the image is analyzed using an image processing algorithm.

In some embodiments, the method further comprises determining an invasion distance of a tumor cell, quantifying network structures formed by the tumor cells, determining the length of network structures formed by the tumor cells, and or/determining the shape of a tumor cell.

In particular embodiments, the 3D collagen matrix comprises, consists of, or consists essentially of about 2 mg/mL to about 6 mg/mL collagen and at least 4 mg/mL PEG.

In some embodiments, the method further comprises determining a gene expression level of one or more genes of a VM gene module in the tumor sample.

In some embodiments, the tumor sample is a biopsy tissue sample or a circulating tumor cell liquid biopsy sample.

In another aspect, provided herein is a culture system comprising, consisting of, or consisting essentially of cells embedded in a high density 3D collagen matrix.

In some embodiments, the collagen density of the high density 3D collagen matrix is selected from the group of, from about 4 mg/mL to about 10 mg/mL, from about 4 mg/mL to about 8 mg/mL, or from about 4 mg/mL to about 6 mg/mL. In a particular embodiment, the collagen density is about 6 mg/mL.

In some embodiments, the 3D collagen matrix comprises a median fiber length less than or equal to 9.5 μm. In some embodiments, the 3D collagen matrix comprises a median pore size less than or equal to 10 μm.

In some embodiments, the 3D collagen matrix further comprises a molecular crowding agent. In some aspects it is selected from polyethylene glycol (PEG), polyvinyl alcohol, dextran and ficoll. In some embodiments, the molecular crowding agent is polyethylene glycol (PEG). In some embodiments, the 3D collagen matrix comprises from about 2 mg/mL to about 6 mg/mL collagen and at least 0.5 mg/mL PEG. In particular embodiments, the 3D collagen matrix comprises from about 2 mg/mL to about 4 mg/mL collagen and at least 4 mg/mL PEG.

When a person is diagnosed with a solid tumor, a gene expression test would be performed and the state of expression of the genes included in the gene set would be assessed as low or high. If the level of expression is high, a recommendation of an additional therapy to surgical resection, or a more aggressive treatment regimen and more careful follow-up would be recommended by the treating physician because the patient is high risk for metastasis.

Thus, in one aspect, the present disclosure provides methods of predicting prognosis in a cancer patient comprising, or alternatively consisting essentially of, or yet further consisting of, determining the expression level of at least a subset of genes in a vascular mimicry (VM) gene module, wherein increased expression of the genes in the VM gene module is indicative of a poor or poorer prognosis.

In some embodiments, the patient has stage I or stage II cancer.

In some embodiments, the cancer is selected from the group consisting of breast cancer, glioma, cervical squamous cell carcinoma, endocervical adenocarcinoma, lung adenocarcinoma, kidney renal clear cell carcinoma, and pancreatic adenocarcinoma.

In some embodiments, the poor prognosis comprises, consists of, or consists essentially of a decreased 5-year survival or increased chance of metastasis.

In some embodiments, the methods further comprising detecting the pore size of the collagen in a tumor sample obtained from the patient or determining the expression level of β1 integrin in a tumor sample from the patient relative to a control level.

The disclosed methods are applicable to all ages, races, and genders of subjects or patients with cancer. Thus, in some embodiments of the disclosed methods the subject is a pediatric subject, while is some embodiments, the subject is an adult.

The foregoing general description and following brief description of the drawings and the detailed description are exemplary and explanatory only. Other objects, advantages, and novel features of the disclosure will be readily apparent to those skilled in the art from the following detailed description.

### BRIEF DESCRIPTION OF DRAWINGS

FIGS. 1A-1I shows high density 3D collagen microenvironment promotes a switch to persistent cell migration in cancer cells. FIG. 1A. Total invasion distance of single cells and their progeny for MDA-MB-231 breast cancer cells in 6 mg/mL (left) and 2.5 mg/mL (right) collagen gels in units of cell length after 48 h of cell encapsulation. FIG. 1B. Mean Squared Displacement (MSD) and persistent time of MDA-MB-231 cells before and after cell division for cells in low density and high density collagen. MSDs are shown for 12 representative cell trajectories. FIG. 1C. Single cell velocity measured at 2 min intervals before and after cell division. Persistence random walk model (PRW model) persistence time computation is described herein. FIG. 1D. Single cell net invasion distance before and after cell division. FIG. 1E. Dot plot showing pore size of 2.5 mg/mL and 6 mg/mL collagen gels as measured from confocal reflection images. FIG. 1F. Representative image of MDA-MB-231 cells cultured in a 6 mg/mL (left) and in a 2.5 mg/mL collagen I matrix after 7 days of culture. Cells are stained with Alexa-488 Phalloidin (F-Actin) and DAPI (nuclei). Scale bar 250 μm. FIG. 1G. Quantification mean structure length from images acquired in 3 independent experiments. FIG. 1H. Representative bright field image of a MDA-MB-231 cells cultured in a 6 mg/mL collagen I matrix where tube like structures and spheroids are in the same field of view. Scale bar 100 μm. FIG. 1I. Quantification of the number of tube-like structures and spheroids in 6 mg/mL collagen I cultured cells. * p<0.05 ** p<0.01 *** p<0.001.

FIGS. 2A-2I shows transcriptomic analysis of cancer cells cultured in low and high density 3D collagen environments shows the upregulation of a gene module related to vascular development. FIG. 2A. Schematic of the experimental approach. FIG. 2B. Principal component analysis of raw RNASeq data shows cell type as main driver of variance in gene expression. FIG. 2C. Principal component analysis of z-score transformed data shows culture condition as the main driver of variance in gene expression. FIG. 2D. Venn diagram showing the overlap between genes upregulated in 6 mg/mL vs 2.5 mg/mL collagen in the 3 cell lines analyzed. FIG. 2E. Bar plot showing mean expression values of the 70 genes identified to be shared uniquely by cancer cell lines. MDA-MB-231 (top), genes sorted by low to high level of expression. HT1080 (bottom) gene order from top panel FIG. 2F. Gene ontology (GO) of biological processes enriched in the 70 genes upregulated by cancer cells in 6 mg/mL collagen. FIG. 2G. Immunofluorescence staining of Collagen type IV of MDA-MB-231 cells after 7 days of culture in 6 mg/mL vs 2.5 mg/mL. Representative images of n=2 biological replicates. Bar graph shows mean and SEM of quantification of stained area performed in 15 different fields of view. Scale bar 100 μm FIG. 2H. Bar plot showing mean expression values of the 35 genes shared by cancer cells and HFF-1 fibroblasts. MDA-MB-231 (top), genes sorted by low to high level of expression. HT1080 (middle) and HFF-1 (bottom) gene order from top panel. FIG. 2I. Gene ontology (GO) of biological processes enriched in the 35 genes shared by cancer cells and HFF-1 fibroblasts.

FIGS. 3A-3K shows the role of the 3D collagen microenvironment on the triggering of vascular mimicry. A. HIF1a expression in low density and high density 3D collagen after 7 days of culture under normoxic (21% O₂) or hypoxic (1% O₂) conditions. FIG. 3B. Images of MDA-MB-231 cells in low density and high density 3D collagen after 7 days of culture under normoxic (21% O₂) or hypoxic (1% O₂) conditions, scale bar 250 μm. FIG. 3C. Quantification of mean structure length in the culture conditions shown in B. FIG. 3D. Storage modulus of collagen gels as estimated by shear rheology during polymerization at different temperatures. FIG. 3E. Images of cells after 7 days of culture in low density collagen polymerized at 37 °C (low stiffness, ~50 Pa) or 20 °C (high stiffness, ~450 Pa). FIG. 3F. Confocal reflection images of 3D matrices. Left: 2.5 mg/mL collagen I, center: 6 mg/mL collagen I and right: 2.5 mg/mL collagen+10 mg/mL PEG. Insert shows a 2× Zoom. Scale bar 100 μm. FIG. 3G. Quantification of pore size in the 2.5 mg/mL collagen+10 mg/mL peg 3D matrix (compare to FIG. 1E). FIG. 3H. Fiber length FIG. 3I. Fiber width FIG. 3J. Representative image of MDA-MB-231 cells cultured for 7 days in a 2.5 mg/mL collagen+10 mg/mL Peg 3D matrix. Cells are stained with Alexa-488 Phalloidin (F-Actin) and DAPI (nuclei). Scale bar 250 μm. FIG. 3K. Representative bright field image of a MDA-MB-231 breast cancer cells cultured in a 2.5 mg/mL collagen matrix where 10 mg/mL peg were added to the media after polymerization.

FIGS. 4A-4C shows the role of β1 Integrin expression on the development of vascular mimicry phenotype as a response to 3D collagen microenvironment. FIG. 4A. Western blot analysis of β1 Integrin expression in MDA-MB-231 cells after CRISPR-Cas9 mediated Knock out of the ITGB1 gene. WT: wild type MDA-MB-231 cell line, sg eGFP: cell line stably expressing lentiCRISPR_V2 vector with a single guide RNA targeting eGFP, sg ITGB1_1 and sg_ITGB1_2: cell line stably expressing 2 different single guide RNA sequences targeting the ITGB1 gene. FIG. 4B. Representative bright field images of MDA-MB-231 cells after 7 days of culture in 2.5 mg/mL (top row) and 6 mg/mL (middle row) collagen 3D matrices. Scale bar 250 μm. Bottom row shows high magnification images of WT and ITGB1 reduced expression MDA-MB-231 cells in 6 mg/mL collagen matrices. Scale bar 100 μm. FIG. 4C. Quantification of the number of tube like structures vs. spheroids found in the 6 mg/mL collagen matrices in control conditions and after reduce expression of β1 Integrin

FIGS. 5A-5D shows analysis of the clinical relevance of the vascular mimicry related transcriptomic module using TCGA data. FIG. 5A. Kaplan meier survival analysis of stage I breast cancer patients when the PC1 loadings were used as an expression metagene. High VM refers to the highest metagene expression scores and Low VM to the lowest expression scores.

FIG. 5B. Kaplan meier survival analysis of stage II breast cancer patients when the PC1 loadings were used as an expression metagene. FIG. 5C. Breakdown of survival analysis from stage I breast cancer patients by tumor molecular subtype. FIG. 5D. Breakdown of survival analysis from stage II breast cancer patients by tumor molecular subtype. LuA: luminal A, LuB: luminal B, tn: Triple Negative, her2: HER2+.

FIGS. 6A-6F shows: FIG. 6A. Representative bright field image of MDA-MB-231 cells embedded in a 6 mg/mL collagen gel but in close contact with the coverslip. Scale bar 100 μm FIG. 6B. Representative trajectories of cells embedded in a 6 mg/mL collagen gel but in close contact with the coverslip before and after cell division. The trajectories show no appreciable differences between the cell movement before or after division. FIG. 6C. Mean Squared Displacement (MSD) and persistent time of HT1080 cells before and after cell division for cells in low density and high density collagen. MSDs shown are 12 representative cell trajectories.

FIG. 6 D. Total invasion distance of single cells and their progeny for HFF-1 fibroblasts cells in 6 mg/mL (left) and 2.5 mg/mL (right) collagen gels in units of cell length after 48 h of cell encapsulation. FIG. 6E. Representative bright field images of HT1080 cells after 7 days of culture in 2.5 mg/mL (left) and 6 mg/mL (right) collagen I matrix. Scale bar 250 μm. FIG. 6F. Representative bright field images of HFF-1 fibroblast cells after 7 days of culture in 2.5 mg/mL (left) and 6 mg/mL (right) collagen I matrix. Scale bar 250 μm.

FIGS. 7A-7C shows: FIG. 7A. Expression levels of genes previously reported as being involved in vascular mimicry development but that were not included in the reported 70 genes list. FIG. 7B. Loadings of the first principal component (PC1) in stage I breast cancer patients of the 70 vascular mimicry related genes identified in this study. FIG. 7C. Loadings of the first principal component (PC1) in stage II breast cancer patients of the 70 vascular mimicry related genes identified in this study. FIG. 7D. VM module did not separate patients with better prognosis in late stage tumors.

FIGS. 8A-8J shows high density 3D collagen microenvironment promotes a switch to persistent cell migration in cancer cells. FIG. 8A. Mean Squared Displacement (MSD) and persistent time of MDA-MB-231 cells before and after cell division in high density collagen. The persistent time was calculated from the MSDs using the persistent random walk model. MSDs are shown for 12 representative cell trajectories. FIG. 8B. Mean Squared Displacement (MSD) and persistent time of MDA-MB-231 cells before and after cell division in low density collagen. The persistent time was calculated from the MSDs using the persistent random walk model. MSDs are shown for 12 representative cell trajectories. FIG. 8C. Single cell velocity measured at 2 min intervals before and after cell division. FIG. 8D. Single cell net invasion distance before and after cell division for cells in high density and low density collagen. FIG. 8E. Representative image of MDA-MB-231 cells cultured in a 6 mg mL⁻¹ (left) and in a 2.5 mg mL⁻¹ (right) collagen I matrix after 7 days of culture. Cells are stained with Alexa-488 Phalloidin (F-Actin) and DAPI (nuclei). Scale bar 250 μm. FIG. 8F. Quantification of mean structure length in low and high density collagen, from images acquired in 3 independent experiments. FIG. 8G. PAS stain of MDA-MB-231 cells cultured for 7 days in a 3D collagen gel of high density (left) and low density (right). Scale bar 100 μm FIG. 8H. Immunofluorescence staining of MDA-MB-231 cells for collagen IV after 7 days of culture in 6 mg mL⁻¹ vs 2.5 mg mL⁻¹. Representative images of n=2 biological replicates. Bar graph shows mean and s.e.m of quantification of stained area performed in 15 different fields of view. Scale bar 100 μm. FIG. 8I. MDA-MB-231 cells cultured on top of growth factor reduced matrigel after 24 hours (left) and after 72 hours (right). Scale bar 250 μm FIG. 8J. MDA-MB-231 cells cultured inside growth factor reduced matrigel in 3D culture for 7 days Scale bar 100 μm. Box plots show quartiles of the dataset with whiskers extending to 1^{st} and 3^{rd} quartiles. n=3 biological replicates for all experiments unless otherwise noted. Statistical significance was determined by Mann-Whitney U test and is indicated as *, **, *** for p≤0.05, p≤0.01, p≤0001 respectively.

FIGS. 9A-9H shows the network forming phenotype induced by high density 3D collagen is accompanied by a transcriptional response common to cancer cells. FIG. 9A. Schematic of the experimental approach. Each cell line in each condition was cultured in biological triplicate, and each replicate was sequenced (n=3 for each cell type per condition).

FIG. 9B. List of genes upregulated in each of the cancer cell lines that are known stem cell or differentiation markers. FIG. 9C. Principal component analysis of raw RNASeq data shows cell type as main driver of variance in gene expression. FIG. 9D. Principal component analysis of z-score transformed data shows culture condition as the main driver of variance in gene expression.

FIG. 9E. Venn diagram showing the overlap between genes upregulated in 6 mg mL⁻¹ vs 2.5 mg mL⁻¹ collagen in the 3 cell lines analyzed. FIG. 9F. Gene ontology (GO) of biological processes enriched in the 70 genes upregulated by cancer cells in 6 mg mL⁻¹ collagen. Number at the end of the bars represent number of genes annotated for the particular GO term. FIG. 9G. Lists of genes with annotations relevant to the observed phenotype. Left: Regulation of cell migration. Middle: Regulation of anatomical structure development. Gray shaded region highlights genes annotated for blood vessel development. Right: surface markers. FIG. 9H. Gene ontology (GO) of biological processes enriched in the 35 genes shared by cancer cells and HFF-1 fibroblasts. Number at the end of the bars represent number of genes annotated for the particular GO term.

FIGS. 10A-10K shows cell network formation is not triggered by hypoxia or matrix stiffness but rather by matrix architecture. FIG. 10A. Storage modulus of collagen gels as estimated by shear rheology after polymerization at different temperatures. FIG. 10B. Representative images of cells after 7 days of culture in low density collagen polymerized at 20 °C (high stiffness, 440 Pa). FIG. 10C. HIF1A expression in low density and high density 3D collagen after 7 days of culture under normoxic (21% O₂) or hypoxic (1% O₂) conditions. FIG. 10D. Representative images of MDA-MB-231 cells in low density and high density 3D collagen after 7 days of culture under hypoxic (1% O₂) conditions, scale bar 250 μm. FIG. 10E. Quantification of mean structure length after 7 days of culture under hypoxic (1% O₂) conditions in low and high density collagen. FIG. 10 F. Confocal reflection images of collagen fibers in 3D matrices. Left: 2.5 mg/mL collagen I, center: 6 mg mL⁻¹ collagen I and right: 2.5 mg mL⁻¹ collagen+10 mg mL⁻¹ PEG. Insert shows a 2× Zoom. Scale bar 100 μm. FIG. 10G. Quantification of pore size in the 3 conditions showed in F. FIG. 10H. Fiber length and FIG. 10I. Fiber width as measured from the confocal reflection images in the 3 conditions showed in F. J. Representative image of MDA-MB-231 cells cultured for 7 days in a 2.5 mg mL⁻¹ collagen+10 mg mL⁻¹ PEG 3D matrix. Cells are stained with Alexa-488 Phalloidin (F-Actin) and DAPI (nuclei). Scale bar 250 μm. FIG. 10K. Representative bright field image of MDA-MB-231 breast cancer cells cultured in a 2.5 mg mL⁻¹ collagen matrix where 10 mg mL⁻¹ PEG was added to the media after polymerization. Scale bar 125 μm. Bar graphs represent mean±/−s.d and data in box and whiskers plots is presented using Tukey method. n=3 biological replicates for all experiments unless otherwise noted. Statistical significance was determined by ANOVA (A,C,G,H,I) and Mann-Whitney U test (E) and is indicated as *, **, *** for p≤0.05, p≤0.01, p≤0001 respectively. Bars plots are mean±-standard deviation.

FIGS. 11A-11J shows role of β1 integrin in the formation of cell network structures in high density collagen. FIG. 11A. Schematic of lentiCRISPR V2 vector used for targeting ITGB1 gene and western blot validation of the protein depletion after 7 days of cell transduction. FIG. 11A discloses SEQ ID NOS 1-2, respectively, in order of appearance. FIG. 11B. Comparison of MDA-MB-231 cells WT and ITGB1 depleted in low density 3D collagen. Left: micrographs showing a representative image of a WT cell undergoing mesenchymal migration and an ITGB1-depleted cell undergoing ameboid migration. Right: quantification of mesenchymal vs. ameboid migration within the cell populations. FIG. 11C. Quantification of the effect of ITGB1 depletion on mean cell velocity when cells are cultured in 6 mg mL⁻¹ collagen. FIG. 11D. Cell persistence and FIG. 11E. cell invasion distance. Comparison for C D and E was performed using Mann-Whitney U test. FIG. 11F. MDA-MB-231 WT, ITGB1-depleted, and control sgRNA cell phenotypes after 7 days of culture in low density collagen (top row) and high density collagen (middle row) Scale bar 250 μm. Bottom row shows high magnification micrographs highlighting the difference between chain structures and spheroids. Scale bar 100 μm. FIG. 11G. Quantification of proportional number of structures in each cell line when cultured in high density collagen. FIG. 11H. Fluorescence activated cells sorting (FACS) was used to separate the parental WT MD-MB-231 cell line population into high-ITGB1 and low-ITGB1 expressing populations. FIG. 11I. ITGB 1 high and ITGB1 low cells after 7 days of culture in high density 3D collagen (top row) and low density (bottom row). Scale bar 200 μm. FIG. 11J. RT-qPCR quantification of a small subset of genes identified in the 70 gene module in WT control and ITGB1-silenced cells when cultured in low and high density collagen. Data shows mRNA levels relative to GAPDH and relative to low density collagen level. Statistical significance evaluated between WT and gITGB1 groups, Statistical significance was determined by ANOVA test. Bar graphs represent mean±/−s.d. and data in box and whiskers plots is presented using Tukey method. n=3 biological replicates for all experiments unless otherwise noted. Significance is indicated as *, **, *** for p≤0.05, p<0.01, p≤0001 respectively.

FIGS. 12A-12C shows the transcriptional response module associated with the collagen induced network phenotype (CINP) is predictive of poor prognosis in human tumor datasets. FIG. 12A. Kaplan Meier survival analysis of stage I breast cancer patients from TCGA and FIG. 12B. METABRIC databases, when the PC1 loadings were used as an expression metagene. High CINP refers to the highest metagene expression scores and Low CINP to the lowest expression scores. HR indicates hazard ratio. FIG. 12C. Sections of a primary breast carcinoma displaying the clinical VM phenotype of chain-like cell structures surrounded by a matrix network. Column 1: Red blood cells, stained by an antibody against GYPA, are indicated by arrows. Several red blood cells are traversing the matrix surrounded by cancer cells. Column 2: Tumor cells are negative for CD31 but in healthy tissue, stained regions colocalize to vessel structures. Column 3: Tumor cells stain strongly for glycogen synthase, which likely contributes to generation of a glycogen rich matrix between the chains of cells. Columns 4-6: Tumor cells undergoing VM stain strongly for three of the most upregulated genes in the VM 70 gene module. Image credit for D: Human Protein Atlas, patient ID 1910, available from www.proteinatlas.org.

FIGS. 13A-13I: FIG. 13A. Representative bright field image of MDA-MB-231 cells embedded in a 6 mg/mL collagen gel but in contact with the coverslip. Scale bar 100 μm FIG. 13B. Representative trajectories of cells embedded in a 6 mg/mL collagen gel but in close contact with the coverslip before and after cell division. The trajectories show no appreciable differences between the cell movement before or after division. FIG. 13C. Mean Squared Displacement (MSD) and persistent time of HT-1080 cells before and after cell division for cells in low density and high density collagen. MSDs shown are 12 representative cell trajectories.

FIG. 13D. Total invasion distance of single cells and their progeny for HFF-1 fibroblasts cells in 6 mg/mL (left) and 2.5 mg/mL (right) collagen gels in units of cell length after 48 h of cell encapsulation. FIG. 13E. Representative confocal reflection image showing collagen fibers around a chain structure formed by MDA-MB-231 cells cultured in high density collagen gel for 7 days, dotted lines show the outline of the chain structure. Scale bar 100 μm. FIG. 13F. Representative bright field images of HT-1080 cells after 7 days of culture in 2.5 mg/mL (left) and 6 mg/mL (right) collagen I matrix. Scale bar 250 μm. FIG. 13G. Representative bright field images of HFF-1 fibroblast cells after 7 days of culture in 2.5 mg/mL (left) and 6 mg/mL (right) collagen I matrix. Scale bar 250 μm. FIG. 13H. Mean structure length formed by MDA-MB-231 cells cultured in high density 3D collagen after 7 days under normoxia (21% O₂) or hypoxia (1% O₂). Comparison was performed using Mann-Whitney U test. FIG. 13I. Representative confocal reflection image showing a 2.5 mg/mL collagen gel polymerized at 20 °C. Scale bar 100 μm. Representative images of N=3 biological replicates for all experiments unless otherwise noted. Statistical significance is indicated as *, **, *** for p≤0.05, p≤0.01, p≤0001 respectively.

FIGS. 14A-14D: FIG. 14A. Bar plot showing mean of n=3 expression values of the 70 genes upregulated by both cancer cell lines. MDA-MB-231 (top), genes sorted by low to high level of expression. HT1080 (bottom) gene order from top panel. FIG. 14B. Bar plot showing mean of n=3 expression values of the 35 genes upregulated by cancer cells and HFF-1 fibroblasts. MDA-MB-231 (top), genes sorted by low to high level of expression. HT1080 (middle) and HFF-1 (bottom) gene order from top panel. FIG. 14C. Mean of n=3 expression levels of genes previously reported as being involved in vasculogenic mimicry and upregulated by cancer cells in high density collagen. For this panel TPM>5 was not required for analysis. FIG. 14D. Sensitivity analysis of Gene Ontology Analysis presented in FIG. 2. Left Panel: Plot showing number of genes included in the analysis as a function of fold change threshold (yellow) and fold enrichment of 2 key terms (blood vessel development and regulation of cell migration, blue and green respectively) for the two gene sets cancer specific (70 Genes) and common to all cell lines analyzed (35 genes). Right panel shows the full sensitivity analysis when the fold change threshold is varied from 1.3 to 1.9.

FIGS. 15A-15C: FIG. 15A. ITGB1 sorted MDA-MB-231 cells at day 1 of embedding in high density and low density collagen matrices and plated on tissue culture plastic (2D). Scale bar 200 μm. FIG. 15B. RT-qPCR validation of shRNA mediated knock down of LAMC2 and COL4A1 FIG. 15C. Representative images of MDA-MB-231 cells expressing shRNA constructs against a scramble sequence, COL4A1, or LAMC2 after 7 days of culture in high density collagen Scale bar 200 μm. N=3 biological replicates for all experiments unless otherwise noted. Statistical significance was determined by Wilcoxon rank sum test and is indicated as * ** *** for p≤0.05, p≤0.01, p≤0001 respectively.

FIGS. 16A-16D: FIG. 16A. Loadings of the first principal component (PC1) in stage I breast cancer patients of the 70 CINP associated genes identified in this study. FIG. 16B. Loadings of the first principal component (PC1) in stage II breast cancer patients of the 70 CINP associated genes identified in this study. FIG. 16C. Kaplan Meier survival analysis of stage II breast cancer patients in TCGA (left) and Metabric (right) databases when the PC1 loadings were used as an expression metagene. FIG. 16D. Kaplan Meier plots showing survival prediction by the CINP gene signature in Stage III and Stage IV breast cancer from TCGA data and stage III from metabric.

FIGS. 17A-17B: Uncropped Western blots. FIG. 17A. Integrin B1 Western blot. FIG. 17B. Alpha tubulin western blot.

FIGS. 18A-18I: Fiber topography modulation by molecular crowding. FIG. 18A. Schematic showing how molecular crowding affects matrix polymerization. FIG. 18B.Reflection confocal micrographs of 2.5 mg/ml collagen polymerized without a molecular crowding agent, PO, or with 2-10 mg/ml of 8 kDa PEG as a crowding agent, P2-P10. Scale bar is 200 μm. C. SEM images of a 2.5 mg/mL collagen gel (top left) and 2.5 mg/ml collagen gels polymerized with 10 mg/mL PEG without washing (top middle) or with thorough washing before fixing (top right). Bottom images are magnified versions of top left and right images. FIG. 18D. Characterization of mean fiber length and FIG. 18E. pore size as a function of the extent of crowding. FIG. 18F. Coefficient of variation of fiber length and FIG. 18G. pore size as a function of the extent of crowding. FIG. 18H. Elastic moduli of control and crowded matrices. FIG. 18I. Local moduli of control and crowded matrices measured by AFM. Only significant differences are noted. N=3 replicates for each condition. At least three fields of view were analyzed per replicate. Bar graphs show the mean and standard error of measurements. Statistical significance tested by ANOVA and reported as p<0.001, ***; p<0.01, **; p<0.05, *.

FIGS. 19A-19F: Influence of PEG crowding alone on cell morphology, migration, and viability in 3D. FIG. 19A. Schematic of control experimental setup. PEG or Ficoll was added after collagen polymerization to evaluate potential effects on cell behavior independent of matrix changes. Influence of PEG crowding on FIG. 19B. cell shape and FIG. 19C. cell migration over 15 hrs. FIG. 19D. Representative micrographs of cells after one week in culture showing brightfield (left), live (green) and dead (red) cell staining. Merged image on right. FIG. 19E. Cell proliferation and FIG. 19F. viability evaluated after one week of PEG or Ficoll crowding after polymerization. N=3 biological replicates for each condition. At least 100 cells were analyzed per condition. Bar graphs show the mean and standard error of measurements. Statistical significance tested by ANOVA and reported as p<0.001, ***; p<0.01, **; p<0.05, *.

FIGS. 20A-20C: Influence of crowded collagen fiber architectures on cell shape in 3D. FIG. 20A. Outlines of representative cells in each matrix condition, P0-P10, after 15 hours. FIG. 20B. Mean cell circularity in each matrix construct. FIG. 20C. Coefficient of variation of cell circularity in each matrix construct. N=3 biological replicates for each condition. At least 100 cells were analyzed per condition. Bar graphs show the mean and standard error. Statistical significance tested by ANOVA and reported as p<0.001, ***; p<0.01, **; p<0.05, *

FIGS. 21A-21I: Influence of fiber topography on cell migration behavior in 3D. FIG. 21A. Representative micrographs of cells in each matrix construct after one week. FIG. 21B. Additional multicellular structures observed at low frequency in P8 and P10 conditions. Lobular (left) and acinar (right three images) structures resembling normal breast structures. Rightmost two images show representative acinar structure stained with DAPI (nuclei, blue) and phalloidin (actin, green) and reveal an organized and hollow morphology. FIG. 21C. Frequency of phenotypes observed in each matrix construct. FIG. 21D. Mean, FIG. 21E. median, and FIG. 21F. coefficient of variation of fiber length in each matrix construct plotted against the frequency of the single cell phenotype in each construct. Gray dotted lines indicate fiber length threshold, below which cells transition into multicellular phenotypes. FIG. 21G. Frequency of the single cell phenotype in each matrix construct plotted against the mean cell circularity in each construct. Red dotted line indicates threshold value below which cells transition into multicellular phenotypes. FIG. 21H. Mean and FIG. 21I. median pore area measurements plotted against the frequency of the single cell phenotype. N=3 biological replicates for each measurement. At least 300 cells were analyzed in each condition.

FIGS. 22A-22C: FIG. 22A. Fiber width at PO and P10. FIG. 22B. Average fiber length with PEG on top or no PEG. FIG. 22C. Pore area with PEG on top or no PEG.

FIGS. 23A-23E: FIG. 23A. Mean fiber length versus mean cell circularity. FIG. 23B. Median fiber length versus median cell circularity. FIG. 23C. 75% Fiber length versus 75% cell circularity. FIG. 23D. 25% fiber length versus 25% cell circularity. FIG. 23E. CV Fiber length versus CV cell circularity.

FIGS. 24A-24D: FIG. 24A. Mean pore area versus mean cell circularity. FIG. 24B. Median pore area versus median cell circularity. FIG. 24C. 75% pore area versus 75% cell circularity. FIG. 24D. 25% pore area versus 25% cell circularity.

FIGS. 25A-25D: FIG. 25A. Mean pore area versus mean cell circularity. FIG. 25B. Median pore area versus median pore circularity. FIG. 25C. 75% pore area versus 75% cell circularity. FIG. 25D. 25% pore area versus 25% cell circularity.

FIGS. 26A-26D: FIG. 26A. XY and YZ planar images of the 2.5 mg/mL collagen condition. FIG. 26B. Average fiber length in XY and YZ planes. FIG. 26C. Pore area in XY and YZ planes. FIG. 26D. Images of fibers at PO (first column), P2 (second column), P4 (third column), P6 (fourth column), P8 (fifth column), and P10 (sixth column).

FIG. 27: Pore area (first column), Fiber length (second column), and Fiber width (third column) for PO (first row), P2 (second row), P4 (third row), P6 (fourth row), P8 (fifth row), and P10 (sixth row).

### DETAILED DESCRIPTION

Effectively targeting tumor cell migration behaviors that precede metastatic dissemination could substantially reduce the morbidity and mortality associated with cancer. The assembly of tumor cells into tubular structures mimicking vasculature has been reported across a broad range of solid tumors. Termed vascular mimicry (VM), histological evidence of this behavior is significantly correlated with metastatic dissemination in over 16 different cancer types. Despite the highly conserved nature of this metastatic process, the mechanisms underlying its induction were poorly understood prior to this disclosure. Based on this disclosure, diagnostic biomarkers and therapeutics targeting VM can be developed to impact the treatment and survival of a wide range of cancer patients. Disclosed herein are a set of genes that mediate the development of vasculogenic mimicry in solid tumors. Expression of this gene set was found to be predictive of patient survival in early stages of breast cancer and in 5 other solid tumor types, and therefor is likely predictive of numerous other types of cancer. This highly conserved gene set provides a useful diagnostic tool and a set of potential therapeutic targets.

### DEFINITIONS

It is to be understood that methods are not limited to the particular embodiments described, and as such may, vary. It is also to be understood that the terminology used herein is for the purpose of describing particular embodiments only, and is not intended to be limiting. The scope of the present technology will be limited only by the appended claims.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps.

Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

As used herein, “about” means plus or minus 10%.

As used herein, “optional” or “optionally” means that the subsequently described event or circumstance may or may not occur, and that the description includes instances where said event or circumstance occurs and instances where it does not.

As used herein, the term “cancer” and “tumor” are used interchangeably and refer to a cell, tissue, subject, or patient with a malignant phenotype characterized by the uncontrolled proliferation of malignant cells. The cancer can be metastatic, non-metastatic and pre-clinical. Hallmarks of cancer include self-sufficiency in growth signals, insensitivity to growth-inhibitory (antigrowth) signals, evasion of pro-grammed cell death (apoptosis), limitless replicative potential, sustained angiogenesis, and tissue invasion and metastasis.

Some examples of such cancers include but are not limited to adrenocortical carcinoma; bladder cancer, breast cancer, breast cancer, ductal, breast cancer, invasive intraductal, breast-ovarian cancer, Burkitt's lymphoma, cervical carcinoma, colorectal adenoma, colorectal cancer, colorectal cancer, hereditary nonpolyposis, type 1, colorectal cancer, hereditary nonpolyposis, type 2, colorectal cancer, hereditary nonpolyposis, type 3, colorectal cancer, hereditary nonpolyposis, type 6, colorectal cancer, hereditary nonpolyposis, type 7, dermatofibrosarcoma protuberans, endometrial carcinoma, esophageal cancer, gastric cancer, fibrosarcoma, glioblastoma multiforme, glomus tumors, multiple, hepatoblastoma, hepatocellular cancer, hepatocellular carcinoma, leukemia, acute lymphoblastic, leukemia, acute myeloid, leukemia, acute myeloid, with eosinophilia, leukemia, acute nonlymphocytic, leukemia, chronic myeloid, Li-Fraumeni syndrome, liposarcoma, lung cancer, lung cancer, small cell, lymphoma, non-Hodgkin's, lynch cancer family syndrome II, male germ cell tumor, mast cell leukemia, medullary thyroid, medulloblastoma, melanoma, meningioma, multiple endocrine neoplasia, myeloid malignancy, predisposition to, myxosarcoma, neuroblastoma, osteosarcoma, ovarian cancer, ovarian cancer, serous, ovarian carcinoma, ovarian sex cord tumors, pancreatic cancer, pancreatic endocrine tumors, paraganglioma, familial nonchromaffin, pilomatricoma, pituitary tumor, invasive, prostate adenocarcinoma, prostate cancer, renal cell carcinoma, papillary, familial and sporadic, retinoblastoma, rhabdoid predisposition syndrome, familial, rhabdoid tumors, rhabdomyosarcoma, small-cell cancer of lung, soft tissue sarcoma, squamous cell carcinoma, head and neck, T-cell acute lymphoblastic leukemia, Turcot syndrome with glioblastoma, tylosis with esophageal cancer, uterine cervix carcinoma, colon-rectal cancer, lung cancer, prostate cancer, skin cancer, osteocarcinoma, solid tumors/malignancies, myxoid and round cell carcinoma, locally advanced tumors, human soft tissue carcinoma, cancer metastases, squamous cell carcinoma, esophageal squamous cell carcinoma, oral carcinoma, cutaneous T cell lymphoma, Hodgkin's lymphoma, non-Hodgkin's lymphoma, cancer of the adrenal cortex, ACTH-producing tumors, non-small cell cancers, gastrointestinal cancers, urological cancers, malignancies of the female genital tract, malignancies of the male genital tract, kidney cancer, brain cancer, bone cancers, skin cancers, thyroid cancer, retinoblastoma, peritoneal effusion, malignant pleural effusion, mesothelioma, Wilms's tumors, gall bladder cancer, trophoblastic neoplasm, hemangiopericytoma, Kaposi's sarcoma and liver cancer.

The term “metastatic cells” refers to cancerous cells that have acquired the ability to migrate from the primary or original tumor lesion to surrounding tissues and/or have acquired the ability to penetrate and the walls of lymphatic cells or blood vessels and circulate through the bloodstream. The term “metastasis” as used herein refers to the migration or spread of cancerous cells from one location in the body to surrounding tissues, the lymphatic system, or to blood vessels. When tumor cells metastasize, the new tumor is referred to as a metastatic tumor.

As used herein, the term “aggressive” in the context of therapy refers to a therapy that is recommended to treat a metastatic tumor. Non-limiting examples of aggressive therapy include chemotherapy and/or radiation therapy. In some embodiments, the aggressive therapy is prophylactic.

The term “chemotherapy” encompasses cancer therapies that employ chemical or biological agents or other therapies, such as radiation therapies, e.g., a small molecule drug or a large molecule, such as antibodies, RNAi and gene therapies. Non-limiting examples of chemotherapies are provided below. It should be understood, although not always explicitly stated, that when a particular therapy is noted, the scope of the invention includes equivalents unless excluded.

Topoisomerase inhibitors are agents designed to interfere with the action of topoisomerase enzymes (topoisomerase I and II), which are enzymes that control the changes in DNA structure by catalyzing the breaking and rejoining of the phosphodiester backbone of DNA strands during the normal cell cycle. In one aspect, topoisomerase inhibitors include irinotecan, topotecan, camptothecin and lamellarin D, or compounds targeting topoisomerase IA. In another aspect, topoisomerase inhibitors include etoposide, doxorubicin or compounds targeting topoisomerase II.

Pyrimidine antimetabolite includes, without limitation, fluorouracil (5-FU), its equivalents and prodrugs. In one embodiment, a pyrimidine antimetabolite is a chemical that inhibits the use of a pyrimidine. The presence of antimetabolites can have toxic effects on cells, such as halting cell growth and cell division, so these compounds can be used as chemotherapy for cancer.

Fluorouracil (5-FU) belongs to the family of therapy drugs called pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluroidine), 1-tetrahydrofuranyl-5-fluorouracil (ftorafur), Capecitabine (Xeloda), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487.

“5-FU based adjuvant therapy” refers to 5-FU alone or alternatively the combination of 5-FU with other treatments, that include, but are not limited to radiation, methyl-CCNU, leucovorin, oxaliplatin, irinotecin, mitomycin, cytarabine, levamisole. Specific treatment adjuvant regimens are known in the art as FOLFOX, FOLFOX4, FOLFIRI, MOF (semustine (methyl-CCNU), vincrisine (Oncovin) and 5-FU). For a review of these therapies see Beaven and Goldberg (2006) Oncology 20 (5): 461-470. An example of such is an effective amount of 5-FU and Leucovorin. Other chemotherapeutics can be added, e.g., oxaliplatin or irinotecan.

Capecitabine is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®

A therapy comprising a pyrimidine antimetabolite includes, without limitation, a pyrimidine antimetabolite alone or alternatively the combination of a pyrimidine antimetabolite with other treatments, that include, but are not limited to, radiation, methyl-CCNU, leucovorin, oxaliplatin, irinotecin, mitomycin, cytarabine, levamisole. Specific treatment adjuvant regimens are known in the art as FOLFOX, FOLFOX4, FOLFOX6, FOLFIRI, MOF (semustine (methyl-CCNU), vincrisine (Oncovin) and 5-FU). For a review of these therapies see Beaven and Goldberg (2006) Oncology 20 (5): 461-470. An example of such is an effective amount of 5-FU and Leucovorin. Other chemotherapeutics can be added, e.g., oxaliplatin or irinotecan.

Bevacizumab (BV) is sold under the trade name Avastin® by Genentech. It is a humanized monoclonal antibody that binds to and inhibits the biologic activity of human vascular endothelial growth factor (VEGF). Biological equivalent antibodies are identified herein as modified antibodies which bind to the same epitope of the antigen, prevent the interaction of VEGF to its receptors (Flt01, KDR a.k.a. VEGFR2) and produce a substantially equivalent response, e.g., the blocking of endothelial cell proliferation and angiogenesis. Bevacizumab is also in the class of cancer drugs that inhibit angiogenesis (angiogenesis inhibitors).

Trifluridine/tipiracil (CAS Number 733030-01-8) is sold under the trade name of Lonsurf. It is a combination of two active pharmaceutical ingredients: trifluridine, a nucleoside analog, and tipiracil hydrochloride, a thymidine phosphorylase inhibitor. Trifluridine has the chemical formula C₁₀H₁₁F₃N₂O₅ and is also known as α,α,α-trifluorothymidine; 5-trifluromethyl-2′-deoxyuridine; and FTD5-trifluoro-2′-deoxythymidine (CAS number 70-00-8). Tipiracil has the chemical formula C₉H₁₁ClN₄O₂ and inhibits the enzyme thymidine phosphorylase, preventing rapid metabolism of trifluridine, increasing the bioavailability of trifluridine. Equivalents of trifluridine/tipiracil include trifluridine alone, trifluridine that modified to increase its halflife and/or resistance to metabolism by thymidine phosphorylase, or substitution of one or both of trifluridine and/or tipiracil hydrochloride with a chemical equivalent. Non-limiting examples of chemical equivalents include pharmaceutically acceptable salts or solvates of the active ingredients.

Irinotecan (CPT-11) is sold under the trade name of Camptosar®. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation. An equivalent of irinotecan is a composition that inhibits a topoisomerase. Non-limiting examples of an equivalent of irinotecan include topotecan, camptothecin and lamellarin D, etoposide, or doxorubicin.

Oxaliplatin (trans-/-diaminocyclohexane oxalatoplatinum; L-OHP; CAS No. 61825-94-3) is sold under the trade name of Elotaxin. It is a platinum derivative that causes cell cytotoxicity. Oxaliplatin forms both inter- and intra-strand cross links in DNA, which prevent DNA replication and transcription, causing cell death. Non-limiting examples of an equivalent of oxaliplatin include carboplatin and cisplatin.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at cancer.gov. Typically, a patient is given a subsequent chemotherapy regimen because the patient did not shown a positive clinical or sub-clinical response to the first line therapy or the first line therapy has stopped.

The term “treating” as used herein is intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, reduction in tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs.

“An effective amount” or “therapeutically effect amount” intends to indicate the amount of a compound or agent administered or delivered to the patient which is most likely to result in the desired response to treatment. The amount is empirically determined by the patient's clinical parameters including, but not limited to the Stage of disease, age, gender, histology, and likelihood for tumor recurrence.

As used herein, “subject” and “patient” are used interchangeably and intend an animal subject or patient, a subject or mammal patient or yet further a human subject or patient. For the purpose of illustration only, a mammal includes but is not limited to a simian, a murine, a bovine, an equine, a porcine or an ovine subject.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), objective response rate (RR or ORR), toxicity or side effect.

“Overall Survival” (OS) refers to the length of time of a cancer patient remaining alive following a cancer therapy. OS is an example of an indication of prognosis.

“Progression free survival” (PFS) or “Time to Tumor Progression” (TTP) refers to the length of time following a therapy, during which the tumor in a subject or cancer patient does not grow. Progression-free survival includes the amount of time a patient has experienced a complete response, partial response or stable disease. PFS and TTP are indications of prognosis.

“Disease free survival” (DFS) refers to the length of time following a therapy, during which a subject or cancer patient survives with no signs of the cancer or tumor. DFS is an indication of prognosis.

“Time to Tumor Recurrence (TTR)” refers to the length of time, following a cancer therapy such as surgical resection or chemotherapy, until the tumor has reappeared (come back). The tumor may come back to the same place as the original (primary) tumor or to another place in the body. TRR is an indication of prognosis.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

“Objective response rate” refers to the proportion of responders (subjects or patients with either a partial (PR) or complete response (CR)) compared to nonresponders (subjects or patients with either SD or PD). Response duration can be measured from the time of initial response until documented tumor progression.

The term “identify” or “identifying” is to associate or affiliate a subject or patient closely to a group or population of subjects or patients who likely experience the same or a similar clinical response to a therapy, or who likely experience the same or a similar cancer pathology such as metastasis.

The term “selecting” a subject or patient for a therapy or treatment refers to making an indication that the selected patient is suitable for the therapy or treatment. Such an indication can be made in writing by, for instance, a handwritten prescription or a computerized report making the corresponding prescription or recommendation.

“Detecting” as used herein refers to determining the presence of a nucleic acid of interest (e.g., at least a subset of the VM biomarker gene signature identified as predictive of poor long-term survival and increased likelihood of metastasis) in a sample. Detection does not require the method to provide 100% sensitivity. Various means of detection are known in the art.

As used herein, the term “sample,” “test sample,” “test genomic sample” or “biological sample” refers to any liquid or solid material derived from an individual believed to have or having cancer. In some embodiments, a test sample is obtained from a biological source, such as cells in culture or a tissue or fluid sample from an animal, most preferably, a human. Exemplary samples include any sample containing the nucleic acid (e.g., DNA or RNA) of interest and include, but are not limited to, a tumor, a circulating tumor cell, cell free DNA (cfDNA), biopsy, aspirates, plasma, serum, whole blood, blood cells, lymphatic fluid, cerebrospinal fluid, synovial fluid, urine, saliva, and skin or other organs (e.g. biopsy material including tumor or bone marrow biopsy). The term “patient sample” as used herein may also refer to a tissue sample obtained from a human seeking diagnosis or treatment of cancer or a related condition or disease. It is also understood that these terms can encompass a population of purified cancer or pre-cancerous cells or a mixture of normal and cancer/precancerous cells. Each of these terms may be used interchangeably.

As used herein, the terms “individual”, “patient”, or “subject” can be an individual organism, a vertebrate, a mammal (e.g., a bovine, a canine, a feline, or an equine), or a human. In a preferred embodiment, the individual, patient, or subject is a human. In the case of human subjects, a pediatric subject is under 18 years of age and an adult subject is 18 years of age or older. A subject is still considered a pediatric subject if he or she begins a course of treatment prior to turning about 18 years of age, even if the subject continues treatment beyond 18 years of age.

As used herein, “having an increased risk” means a subject is identified as having a higher than normal chance of developing metastasis and/or metastatic cancer, compared to the average cancer patient. In addition, a subject who has had, or who currently has, cancer is a subject who has an increased risk for developing cancer, as such a subject may continue to develop cancer. Subjects who currently have, or who have had, a tumor also have an increased risk for tumor metastases.

As used herein, “determining a prognosis” refers to the process in which the course or outcome of a condition in a patient is predicted. The term “prognosis” does not refer to the ability to predict the course or outcome of a condition with 100% accuracy. Instead, the term refers to identifying an increased or decreased probability that a certain course or outcome will occur in a patient exhibiting a given condition/marker, when compared to those individuals not exhibiting the condition. The nature of the prognosis is dependent upon the specific disease and the condition/marker being assessed. For example, a prognosis may be expressed as the amount of time a patient can be expected to survive, the likelihood that the disease goes into remission or experience recurrence, or to the amount of time the disease can be expected to remain in remission before recurrence.

“Expression” as applied to a gene, refers to the production of the mRNA transcribed from the gene, or the protein product encoded by the gene. The expression level of a gene may be determined by measuring the amount of mRNA or protein in a cell or tissue sample. In one aspect, the expression level of a gene is represented by a relative level as compared to a housekeeping gene as an internal control. In another aspect, the expression level of a gene from one sample may be directly compared to the expression level of that gene from a different sample using an internal control to remove the sampling error.

The expression “amplification of polynucleotides” includes methods such as PCR, ligation amplification (or ligase chain reaction, LCR) and amplification methods based on the use of Q-beta replicase. These methods are well known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

A “normal cell or tissue corresponding to the tumor tissue type” refers to a normal cell or tissue from a same tissue type as the tumor tissue. A non-limiting examples is a normal lung cell from a patient having lung tumor, or a normal colon cell from a patient having colon tumor.

The term “amplification” or “amplify” as used herein means one or more methods known in the art for copying a target nucleic acid, thereby increasing the number of copies of a selected nucleic acid sequence. Amplification can be exponential or linear. A target nucleic acid can be either DNA or RNA. The sequences amplified in this manner form an “amplicon.” While the exemplary methods described hereinafter relate to amplification using the polymerase chain reaction (“PCR”), numerous other methods are known in the art for amplification of nucleic acids (e.g., isothermal methods, rolling circle methods, etc.). The skilled artisan will understand that these other methods can be used either in place of, or together with, PCR methods.

As used herein the term “stringency” is used in reference to the conditions of temperature, ionic strength, and the presence of other compounds, under which nucleic acid hybridizations are conducted. With high stringency conditions, nucleic acid base pairing will occur only between nucleic acids that have sufficiently long segments with a high frequency of complementary base sequences. Exemplary hybridization conditions are as follows. High stringency generally refers to conditions that permit hybridization of only those nucleic acid sequences that form stable hybrids in 0.018 M NaCl at 65 °C. High stringency conditions can be provided, for example, by hybridization in 50% formamide, 5×Denhardt's solution, 5×SSC (saline sodium citrate) 0.2% SDS (sodium dodecyl sulfate) at 42 °C, followed by washing in 0.1×SSC, and 0.1% SDS at 65 °C. Moderate stringency refers to conditions equivalent to hybridization in 50% formamide, 5×Denhardt's solution, 5×SSC, 0.2% SDS at 42 °C, followed by washing in 0.2×SSC, 0.2% SDS, at 65 °C. Low stringency refers to conditions equivalent to hybridization in 10% formamide, 5×Denhardt's solution, 6×SSC, 0.2% SDS, followed by washing in 1° SSC, 0.2% SDS, at 50 °C.

As used herein the term “substantially identical” refers to a polypeptide or nucleic acid exhibiting at least 50%, 75%, 85%, 90%, 95%, or even 99% identity to a reference amino acid or nucleic acid sequence over the region of comparison. For polypeptides, the length of comparison sequences will generally be at least 20, 30, 40, or 50 amino acids or more, or the full length of the polypeptide. For nucleic acids, the length of comparison sequences will generally be at least 10, 15, 20, 25, 30, 40, 50, 75, or 100 nucleotides or more, or the full length of the nucleic acid.

As used herein, a “molecular crowding agent” or “crowding agent” refers to an agent capable of providing molecular crowding to the 3D collagen matrix. Nonlimiting examples include one or more of: polyethylene glycol (e.g., PEG1450, PEG3000, PEG8000, PEG10000, PEG14000, PEG15000, PEG20000, PEG250000, PEG30000, PEG35000, PEG40000, PEG compound with molecular weight between 15,000 and 20,000 daltons, or combinations thereof), polyvinyl alcohol, dextran and ficoll. In some embodiments, the crowding agent is present in the reaction mixture at a concentration between 1 to 12% by weight or by volume of the reaction mixture, e.g., between any two concentration values selected from 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, and 12.0%. In particular embodiments, the molecular crowding agent is PEG.

### Methods of Predicting Prognosis and Likelihood of Metastasis

This disclosure provides methods and kits for predicting the prognosis of cancer patients and the likelihood of metastasis of a given caner, which is useful in stratifying patients and identifying/differentiating between aggressive and indolent disease. The disclosed kits and methods may further be useful for selecting a therapeutic regimen or determining if a certain therapeutic regimen is more likely to treat a cancer or is the appropriate chemotherapy for that patient than other chemotherapies that may be available to the patient. In general, a therapy is considered to “treat” cancer if it provides one or more of the following treatment outcomes: reduce or delay recurrence of the cancer after the initial therapy; increase median survival time or decrease metastases.

An initial step in cancer metastasis is the migration of tumor cells through extracellular matrix (ECM) and into the lymphatic or vascular systems. Several distinct cancer cell migration strategies exist in vivo, and the local density and alignment of collagen are implicated in modulating these migration behaviors. Yet, clonal cells within a tumor population also display heterogeneity in their ability to migrate and metastasize. Prior to this disclosure, it remained unclear to what extent tumor cell heterogeneity versus ECM heterogeneity contribute to the emergence of distinct migration phenotypes. This disclosure has identified such a phenotype through the use of a 3D collagen system to generate matrices of varying densities and monitored single cancer cell migration in these matrices with time-lapse microscopy. The existence of a collagen density threshold at 2.5 mg/ml, above which 86% of MDA-MB-231 breast cancer cells transition from single mesenchymal migration to collective cell migration was observed. Initially embedded as single cells, the majority of MDAs in 6 mg/ml collagen began migrating collectively with a 50% increase in persistence after cell division. The remainder of cells did not migrate, but instead formed spheroids. Conversely, in 2.5 mg/ml collagen, cells migrated individually. Moreover, highly similar behavior in HT-1080 fibrosarcoma cells was observed.

Within seven days, cells in 6 mg/ml undergoing collective motility created long interconnected networks coated with basement membrane molecules that resembled a clinical phenotype known as vascular mimicry (VM). Next the physical feature of high density collagen driving VM was identified. Compared to the 2.5 mg/ml condition, 6 mg/ml collagen corresponded to an increased stiffness and adhesive ligand concentration as well as decreased oxygen concentration and pore size. To test these features individually, cell were cultured in temperature stiffened 2.5 mg/ml matrices, or in 2.5 mg/ml with 1% oxygen, with integrin activating antibodies, or with a high density of polyethylene glycol (PEG). Neither hypoxia, matrix stiffness, or integrin activation was sufficient to induce VM. However, PEG-induced molecular crowding triggered VM network formation. Moreover, RNA sequencing revealed that cells undergoing collective migration up-regulated a conserved transcriptional program consisting of 70 genes. This gene set was not up-regulated in normal mesenchymal fibroblasts under the same conditions. Further analysis showed that this gene module was significantly enriched for annotations of vascular development and negative motility regulation and predicted survival in human tumor transcriptome datasets. Together, the disclosed results indicate that the VM phenotype arises in a subpopulation of cells from a conserved transcriptional and migratory response to molecular crowding in 3D.

Existing gene sets used as cancer diagnostic tools (i.e. OncoDX and MammaPrint) compile genes involved in many different aspects of cancer biology without any link to a functional phenotype. The gene set presented in this disclosure has been validated to be linked to the development of VM, a specific and highly aggressive metastatic cancer cell phenotype. Currently, VM is identified by a pathologist's evaluation of histological slides, wherein vascular-like structures that do not stain positive for endothelial cells are identified as VM. Thus far, conserved molecular biomarkers that define this phenotype have remained unknown. The disclosed discovery informs a universal set of VM diagnostic biomarkers for improving assignment of patients to therapies, which may be useful for diseases like ductal carcinoma in situ and prostate cancers that are frequently over-treated due to an inability to distinguish indolent from aggressive disease. Moreover, this disclosure will inform potential therapeutic strategies for combatting VM-mediated metastasis.

Thus, the present disclosure provides methods of detecting a novel VM gene module made up of the 70 up-regulated genes shown in Table 1 below. Detection of the expression level of these genes can be used to estimate the risk of tumor metastasis in a subject and/or the prognosis of a cancer patient. Up-regulation or increased expression of the genes in the gene module can be relative to a defined control level. The control level may be determined by detecting expression levels of the genes in a non-cancerous sample from the patient or based on expression data in the general population.

TABLE 1
VM Module Genes, ranked

| | | | Entrez | |
|---|---|---|---|---|
| Rank | Gene | Gene Name | Gene Ref. | Transcript Refs. |
| 1 | COL5A1 | Collagen | 1289 | NM_000093 |
| | | alpha-1(V) chain | | NM_001278074 |
| 2 | FRMD6 | FERM domain- | 122786 | NM_001042481 |
| | | containing | | NM_001267046 |
| | | protein 6 | | NM_001267047 |
| | | | | NM_152330 |
| 3 | TANC2 | Tetratricopeptide | 26115 | NM_025185 |
| | | Repeat, | | |
| | | Ankyrin Repeat | | |
| | | And Coiled- | | |
| | | Coil Containing 2 | | |
| 4 | THBS1 | Thrombospondin 1 | 7057 | NM_003246 |
| 5 | PEAK1 | Pseudopodium | 79834 | NM_024776 |
| | | Enriched | | |
| | | Atypical Kinase 1 | | |
| 6 | ITGAV | Integrin alpha-V | 3685 | NM_001144999 |
| | | | | NM_001145000 |
| | | | | NM_002210 |
| 7 | DAAM1 | Disheveled- | 23002 | NM_001270520 |
| | | associated | | NM_014992 |
| | | activator | | |
| | | of morphogenesis 1 | | |
| 8 | RASEF | Ras and EF-hand | 158158 | NM_152573 |
| | | domain- | | |
| | | containing protein | | |
| 9 | JAG1 | Jagged1 | 182 | NM_000214 |
| 10 | LAMC2 | Laminin subunit | 3918 | NM_018891 |
| | | gamma-2 | | NM_005562 |
| 11 | ZNF532 | Zinc finger | 55205 | NM_018181 |
| | | protein 532 | | NM_001318726 |
| | | | | NM_001318727 |
| | | | | NM_001318728 |
| | | | | NM_001353525 |
| 12 | SKIL | Ski-like protein | 6498 | NM_001145097 |
| | | | | NM_001145098 |
| | | | | NM_001248008 |
| | | | | NM_005414 |
| 13 | NAV1 | Neuron navigator 1 | 89796 | NM_001167738 |
| | | | | NM_020443 |
| 14 | ARHGAP32 | Rho GTPase- | 9743 | NM_001142685 |
| | | activating protein | | NM_014715 |
| | | 32 | | |
| 15 | SYNE1 | Enaptin | 23345 | NM_001099267 |
| | | | | NM_001134379 |
| | | | | NM_015293 |
| | | | | NM_033071 |
| | | | | NM_133650 |
| 16 | GALNT10 | Polypeptide N- | 55568 | NM_017540 |
| | | Acetylgalacto- | | NM_198321 |
| | | saminyltransferase | | NM_024564 |
| | | 10 | | |
| 17 | LHFPL2 | Lipoma HMGIC | 10184 | NM_005779 |
| | | fusion partner- | | |
| | | like 2 protein | | |
| 18 | ABL2 | Tyrosine-protein | 27 | NM_001136000 |
| | | kinase ABL2 | | NM_001136001 |
| | | | | NM_001168236 |
| | | | | NM_001168237 |
| | | | | NM_001168238 |
| | | | | NM_001168239 |
| | | | | NM_005158 |
| | | | | NM_007314 |
| 19 | LTBP1 | Latent | 4052 | NM_206943 |
| | | transforming growth | | |
| | | factor beta | | |
| | | binding protein 1 | | |
| 20 | COL4A1 | Collagen | 1282 | NM_001845 |
| | | alpha-1(IV) chain | | NM_001303110 |
| 21 | DPY19L1 | Dpy-19 Like C- | 23333 | NM_015283 |
| | | Mannosyltransferase | | |
| | | 1 | | |
| 22 | LPCAT2 | Lysophosphatidyl- | 54947 | NM_017839 |
| | | choline | | NM_032330 |
| | | Acyltransferase 2 | | |
| 23 | TBC1D2B | TBC1 Domain | 23102 | NM_015079 |
| | | Family Member | | NM_144572 |
| | | 2B | | |
| 24 | LAMB1 | Laminin subunit | 3912 | NM_002291 |
| | | beta-1 | | |
| 25 | AMIGO2 | Adhesion Molecule | 347902 | NM_001143668 |
| | | With Ig | | NM_181847 |
| | | Like Domain 2 | | |
| 26 | NREP | Neuronal | 9315 | NM_004772 |
| | | Regeneration | | NM_001142478 |
| | | Related | | |
| | | Protein | | |
| 27 | SNX30 | Sorting Nexin | 401548 | NM_001012994 |
| | | Family | | |
| | | Member 30 | | |
| 28 | TPM1 | Tropomyosin | 7168 | NM_000366 |
| | | alpha-1 chain | | NM_001018004 |
| | | | | NM_001018005 |
| | | | | NM_001018006 |
| | | | | NM_001018007 |
| | | | | NM_001018008 |
| | | | | NM_001018020 |
| | | | | NM_001301244 |
| | | | | NM_001301289 |
| | | | | NM_001330344 |
| | | | | NM_001330346 |
| | | | | NM_001330351 |
| 29 | COL4A2 | Collagen | 1284 | NM_001846 |
| | | alpha-2(IV) | | |
| | | chain | | |
| 30 | ARNTL | Aryl hydrocarbon | 406 | NM_001030272 |
| | | receptor | | NM_001030273 |
| | | nuclear translocator- | | NM_001178 |
| | | like protein | | NM_001297719 |
| | | 1 | | NM_001297722 |
| | | | | NM_001297724 |
| 31 | MRC2 | mannose receptor, | 9902 | NM_006039 |
| | | C type 2 | | |
| 32 | TGFBI | Transforming | 7045 | NM_000358 |
| | | growth factor, | | |
| | | beta-induced, | | |
| | | 68 kDa | | |
| 33 | TVP23C | Trans-Golgi | 201158 | NM_001135036 |
| | | Network Vesicle | | NM_145301 |
| | | Protein 23 | | |
| | | Homolog C | | |
| 34 | BHLHE40 | Basic Helix-Loop- | 8553 | NM_003670 |
| | | Helix Family | | |
| | | Member E40 | | |
| 35 | SMAD7 | Mothers against | 4092 | NM_005904 |
| | | decapentaplegic | | NM_001190821 |
| | | homolog 7 | | NM_001190822 |
| | | | | NM_001190823 |
| 36 | ABLIM3 | Actin-binding | 22885 | NM_001301015 |
| | | LIM protein 3 | | NM_001301018 |
| | | | | NM_001301027 |
| | | | | NM_001301028 |
| | | | | NM_014945 |
| | | | | NM_001345858 |
| | | | | NM_001345859 |
| | | | | NM_001345860 |
| | | | | NM_001345861 |
| 37 | ZNF224 | Zinc finger | 7767 | NM_013398 |
| | | protein 224 | | NM_001321645 |
| 38 | PODXL | Podocalyxin-like | 5420 | NM_005397 |
| | | protein 1 | | NM_001018111 |
| 39 | TAGLN | Transgelin | 6876 | NM_003186 |
| | | | | NM_001001522 |
| 40 | VHL | von Hippel- | 7428 | NM_000551 |
| | | Lindau tumor | | NM_198156 |
| | | suppressor | | NM_001354723 |
| 41 | EPHB2 | Ephrin type-B | 2048 | NM_001309192 |
| | | receptor 2 | | NM_001309193 |
| | | | | NM_004442 |
| | | | | NM_017449 |
| 42 | EDN1 | Endothelin 1 | 1906 | NM_001168319 |
| | | | | NM_001955 |
| 43 | GTF2IP4 | General | 100093631 | NR_003580 |
| | | Transcription | | |
| | | Factor IIi | | |
| | | Pseudogene 4 | | |
| 44 | HPS4 | Hermansky-Pudlak | 89781 | NM_022081 |
| | | syndrome 4 | | NM_152840 |
| | | protein | | NM_152841 |
| | | | | NM_152842 |
| | | | | NM_152843 |
| | | | | NM_001349896 |
| | | | | NM_001349898 |
| | | | | NM_001349899 |
| | | | | NM_001349900 |
| | | | | NM_001349901 |
| | | | | NM_001349902 |
| | | | | NM_001349903 |
| | | | | NM_001349904 |
| | | | | NM_001349905 |
| 45 | SIPA1L1 | Signal-induced | 26037 | NM_001284245 |
| | | proliferation- | | NM_001284246 |
| | | associated 1-like | | NM_001284247 |
| | | protein 1 | | NM_015556 |
| | | | | NM_001354285 |
| | | | | NM_001354286 |
| | | | | NM_001354287 |
| | | | | NM_001354288 |
| | | | | NM_001354289 |
| 46 | PID1 | Phosphotyrosine | 55022 | NM_001100818 |
| | | Interaction | | NM_017933 |
| | | Domain | | |
| | | Containing 1 | | |
| 47 | NLGN2 | Neuroligin-2 | 57555 | NM_020795 |
| 48 | LTBP4 | Latent | 8425 | NM_003573 |
| | | transforming growth | | |
| | | factor beta binding | | |
| | | protein 4 | | |
| 49 | TRMT13 | TRNA | 54482 | NM_019083 |
| | | Methyltransferase | | |
| | | 13 Homolog | | |
| 50 | IGF2BP3 | Insulin-like | 10643 | NM_006547 |
| | | growth factor 2 | | |
| | | mRNA-binding | | |
| | | protein 3 | | |
| 51 | RBPJ | Recombining | 3516 | NM_005349 |
| | | binding protein | | NM_015874 |
| | | suppressor of | | NM_203283 |
| | | hairless | | NM_203284 |
| 52 | MKL1 | MKL/ | 57591 | NM_001282660 |
| | | megakaryoblastic | | NM_001282661 |
| | | leukemia 1 | | NM_001282662 |
| | | | | NM_020831 |
| | | | | NM_001318139 |
| 53 | ZMYM5 | Zinc Finger | 9205 | NM_001142684 |
| | | MYM-Type | | NM_001039650 |
| | | Containing 5 | | NM_014242 |
| 54 | EFCAB11 | EF-Hand | 90141 | NM_145231 |
| | | Calcium Binding | | NM_001284267 |
| | | Domain 11 | | |
| 55 | WDR66 | WD Repeat | 144406 | NM_001178003 |
| | | Domain 66 | | NM_144668 |
| 56 | NKX3-1 | Homeobox protein | 4824 | NM_001256339 |
| | | Nkx-3.1 | | NM_006167 |
| 57 | HMOX1 | HMOX1 | 3162 | NM_002133 |
| | | (heme oxygenase | | |
| | | (decycling) 1) | | |
| 58 | TYRO3 | Tyrosine-protein | 7301 | NM_006293 |
| | | kinase | | NM_001330264 |
| | | receptor TYRO3 | | |
| 59 | SDHAP1 | Succinate | 255812 | AK125217.1 |
| | | Dehydrogenase | | AK299148.1 |
| | | Complex | | AF088032.1 |
| | | Flavoprotein | | |
| | | Subunit | | |
| | | A Pseudogene 1 | | |
| 60 | FURIN | Furin | 5045 | NM_002569 |
| | | | | NM_001289823 |
| | | | | NM_001289824 |
| 61 | FAM43A | Protein FAM43A | 131583 | NM_153690 |
| 62 | AGTRAP | Type-1 angiotensin | 57085 | NM_001040194 |
| | | II receptor- | | NM_001040195 |
| | | associated protein | | NM_001040196 |
| | | | | NM_001040197 |
| | | | | NM_020350 |
| 63 | KCTD11 | Potassium Channel | 147040 | NM_001002914 |
| | | Tetramerization | | |
| | | Domain | | |
| | | Containing 11 | | |
| 64 | ID2 | DNA-binding | 3398 | NM_002166 |
| | | protein inhibitor | | |
| | | ID-2 | | |
| 65 | FERMT1 | Fermitin family | 55612 | NM_017671 |
| | | homolog 1 | | |
| 66 | MTND2P28 | Mitochondrially | 100652939 | ENST00000457540 |
| | | Encoded | | |
| | | NADH:Ubiquinone | | |
| | | Oxidoreductase | | |
| | | Core Subunit 2 | | |
| | | Pseudogene 28 | | |
| 67 | H2BFS | Histone H2B | 54145 | NM_017445 |
| | | type F-S | | |
| 68 | LFNG | LFNG | 3955 | NM_002304 |
| | | O-fucosylpeptide | | NM_001040167 |
| | | 3-beta-N- | | NM_001040168 |
| | | acetylglucosaminyl- | | NM_001166355 |
| | | transferase | | |
| 69 | HES 1 | Transcription | 3280 | NM_005524 |
| | | factor HES1 | | |
| 70 | KIN | DNA/RNA- | 22944 | NM_012311 |
| | | binding protein | | |
| | | KIN17 | | |

Accordingly, in one aspect provided herein is a method of determining gene expression level of one or more genes of a vascular mimicry (VM) gene module in a sample isolated from a subject, comprising, consisting of, or consisting essentially of analyzing the expression of the one or more genes listed in the VM gene module. In some embodiments, the method further comprises determining a risk of tumor metastasis in the subject by comparing a change in expression of the one or more genes in the VM gene module compared to a predetermined reference level.

In another aspect, disclosed herein is a method of predicting prognosis for a cancer patient, the method comprising, consisting of, or consisting essentially of: determining a gene expression level of one or more genes of a vascular mimicry (VM) gene module in a sample isolated from the cancer subject. In some embodiments, the method further comprises identifying the patient as having poor prognosis by comparing a change in expression of the one or more genes in the VM gene module compared to a predetermined reference level. In some embodiments, an increase in expression of the one or more genes in the VM gene module compared to a predetermined reference level is indicative of poor prognosis.

Methods to detect the disclosed VM biomarkers include, but are not limited to using PCR-based methods such as Q-PCR and RT-PCR to determine whether a subject has an increased risk of metastasis or a poor prognosis (i.e. decreased 5-year survival). Alternatively, mRNA levels can be detected using nucleic acid probes or arrays.

In some embodiments, the disclosure relates to methods and compositions for determining and identifying the presence of a VM phenotype based on detecting of the disclosed gene module. This information is useful to diagnose and prognose disease progression as well as select the most effective treatment among treatment options. Probes can be used to directly determine the genotype of the sample or can be used simultaneously with or subsequent to amplification. The term “probes” includes naturally occurring or recombinant single- or double-stranded nucleic acids or chemically synthesized nucleic acids. They may be labeled by nick translation, Klenow fill-in reaction, PCR or other methods known in the art. Probes of the present disclosure, their preparation and/or labeling are described in Sambrook et al. (1989) supra. A probe can be a polynucleotide of any length suitable for selective hybridization to a nucleic acid containing a polymorphic region of the invention. Length of the probe used will depend, in part, on the nature of the assay used and the hybridization conditions employed.

In some embodiments, the one or more genes of the VM gene module comprise, consist of, or consist essentially of at least one, at least two, at least three, at least four, at least five, at least six, at least seven, at least eight, at least nine, at least ten, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, or 70 genes selected from COL5A1, FRMD6, TANC2, THBS1, PEAK1, ITGAV, DAAM1, RASEF, JAG1, LAMC2, ZNF532, SKIL, NAV1, ARHGAP32, SYNE1, GALNT10, LHFPL2, ABL2, LTBP1, COL4A1, DPY19L1, LPCAT2, TBC1D2B, LAMB1, AMIGO2, NREP, SNX30, TPM1, COL4A2, ARNTL, MRC2, TGFBI, TVP23C, BHLHE40, SMAD7, ABLIM3, ZNF224, PODXL, TAGLN, VHL, EPHB2, EDN1, GTF2IP4, HPS4, SIPA1L1, PID1, NLGN2, LTBP4, TRMT13, IGF2BP3, RBPJ, MKL1, ZMYM5, EFCAB11, WDR66, NKX3-1, HMOX1, TYRO3, SDHAP1, FURIN, FAM43A, AGTRAP, KCTD11, ID2, FERMT1, MTND2P28, H2BFS, LFNG, HES1, or KIN.

In some embodiments, the VM gene module comprises, consists of, consists essentially of, or further comprises at least one, at least two, at least three, or four genes selected from ITGB1, LAMC2, COL4A1, and DAAM1.

In some embodiments, probes are labeled with two fluorescent dye molecules to form so-called “molecular beacons” (Tyagi, S. and Kramer, F. R. (1996) Nat. Biotechnol. 14:303-8). Such molecular beacons signal binding to a complementary nucleic acid sequence through relief of intramolecular fluorescence quenching between dyes bound to opposing ends on an oligonucleotide probe. The use of molecular beacons for genotyping has been described (Kostrikis, L. G. (1998) Science 279:1228-9) as has the use of multiple beacons simultaneously (Marras, S. A. (1999) Genet. Anal. 14:151-6). A quenching molecule is useful with a particular fluorophore if it has sufficient spectral overlap to substantially inhibit fluorescence of the fluorophore when the two are held proximal to one another, such as in a molecular beacon, or when attached to the ends of an oligonucleotide probe from about 1 to about 25 nucleotides.

Labeled probes also can be used in conjunction with amplification of a polymorphism. (Holland et al. (1991) Proc. Natl. Acad. Sci. 88:7276-7280). U.S. Pat. No. 5,210,015 by Gelfand et al. describe fluorescence-based approaches to provide real time measurements of amplification products during PCR. Such approaches have either employed intercalating dyes (such as ethidium bromide) to indicate the amount of double-stranded DNA present, or they have employed probes containing fluorescence-quencher pairs (also referred to as the “Taq-Man” approach) where the probe is cleaved during amplification to release a fluorescent molecule whose concentration is proportional to the amount of double-stranded DNA present. During amplification, the probe is digested by the nuclease activity of a polymerase when hybridized to the target sequence to cause the fluorescent molecule to be separated from the quencher molecule, thereby causing fluorescence from the reporter molecule to appear. The Taq-Man approach uses a probe containing a reporter molecule-quencher molecule pair that specifically anneals to a region of a target polynucleotide containing the polymorphism.

Probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayyem et al. U.S. Pat. No. 5,952,172 and by Kelley, S. O. et al. (1999) Nucleic Acids Res. 27:4830-4837.

In addition to methods which focus primarily on the detection of one nucleic acid sequence, profiles can also be assessed in such detection schemes. Fingerprint profiles can be generated, for example, by utilizing a differential display procedure, Northern analysis and/or RT-PCR.

In some detection methods, it is necessary to first amplify at least a portion of the VM gene module (i.e. the genes of interest) prior to identifying the expression level of the genes. Amplification can be performed, e.g., by PCR and/or LCR, according to methods known in the art. In one embodiment, genomic DNA of a sample (e.g. at least one cell from a patient) is exposed to PCR primers and amplification for a number of cycles sufficient to produce the required amount of amplified DNA.

Alternative amplification methods include: self-sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al., (1988) Bio/Technology 6:1197), or any other nucleic acid amplification method, followed by the detection of the amplified molecules using techniques known to those of skill in the art. These detection schemes are useful for the detection of nucleic acid molecules if such molecules are present in very low numbers.

In some embodiments, any of a variety of sequencing reactions known in the art can be used to directly sequence at least a portion of the VM gene module (i.e. the genes of interest). Exemplary sequencing reactions include those based on techniques developed by Maxam and Gilbert ((1997) Proc. Natl. Acad Sci USA 74:560) or Sanger (Sanger et al. (1977) Proc. Nat. Acad. Sci. 74:5463). It is also contemplated that any of a variety of automated sequencing procedures can be utilized when performing the subject assays (Biotechniques (1995) 19:448), including sequencing by mass spectrometry (see, for example, U.S. Pat. No. 5,547,835 and international patent application Publication Number WO94/16101, entitled DNA Sequencing by Mass Spectrometry by H. Koster; U.S. Pat. No. 5,547,835 and international patent application Publication Number WO 94/21822 entitled “DNA Sequencing by Mass Spectrometry Via Exonuclease Degradation” by H. Koster; U.S. Pat. No. 5,605,798 and International Patent Application No. PCT/US96/03651 entitled DNA Diagnostics Based on Mass Spectrometry by H. Koster; Cohen et al. (1996) Adv. Chromat. 36:127-162; and Griffin et al. (1993) Appl Biochem Bio. 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method Of DNA Sequencing Employing A Mixed DNA-Polymer Chain Probe” and U.S. Pat. No. 5,571,676 entitled “Method For Mismatch-Directed In Vitro DNA Sequencing”.

In some embodiments, the gene expression level is determined by a method comprising determining the amount of an mRNA transcribed from the one or more genes of the VM gene module. In some embodiments, the gene expression level is determined by a method comprising, consisting of, or consisting essentially of one or more of in situ hybridization, northern blot, PCR, quantitative PCR, RNA-seq, or microarray. In some embodiments, the change in expression of the genes in the VM gene module is increased compared to the predetermined reference level.

In some embodiments, the sample is a tumor sample. In some embodiments, the tumor sample is at least one of a fixed tissue, a frozen tissue, a biopsy tissue, a circulating tumor cell liquid biopsy, a resection tissue, a microdissected tissue, or a combination thereof. In particular embodiments, the sample is a biopsy tissue sample or a circulating tumor cell liquid biopsy sample.

In some embodiments, the subject has been diagnosed with cancer. In some embodiments, the cancer is a stage I or stage II cancer. In some embodiments, the cancer is selected from breast cancer, glioma, cervical squamous cell carcinoma, endocervical adenocarcinoma, lung adenocarcinoma, kidney renal clear cell carcinoma, and pancreatic adenocarcinoma.

In some embodiments, the method further comprises the step of culturing the sample in a high density 3D collagen culture system and determining the sample's migration capacity. In some embodiments, the method further comprises administering a cancer treatment comprising chemotherapy, that is optionally an aggressive treatment, and/or radiation therapy.

In some embodiments, the subject is a mammal. In some embodiments, the subject is an equine, bovine, canine, feline, murine, or a human. In a particular embodiment, the subject is a human.

Disclosed herein are methods for diagnostic and prognostic evaluation of cancer. Also disclosed are methods of treating cancer. In one aspect, the expression of genes are determined in different subjects for which either diagnosis or prognosis information is desired, in order to provide cancer profiles.

Within the sample, different expression profiles may be indicative of different prognosis states (i.e. good long term survival prospects or poor long term survival prospects, for example). By comparing profiles of cancer tissue in different states, information regarding which genes are important (including both up- and down-regulation of genes of interest) in each of these states is obtained. The identification of sequences that are differentially expressed in cancer tissue, as well as differential expression resulting in different prognostic outcomes is clinically invaluable for determining patient treatment.

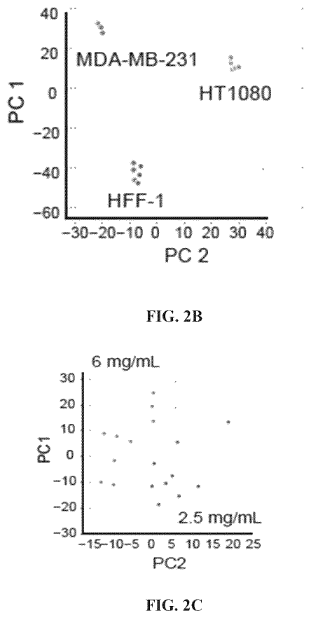

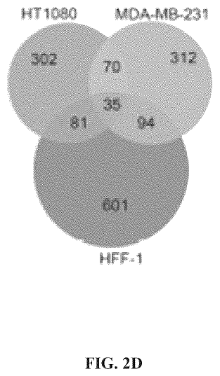

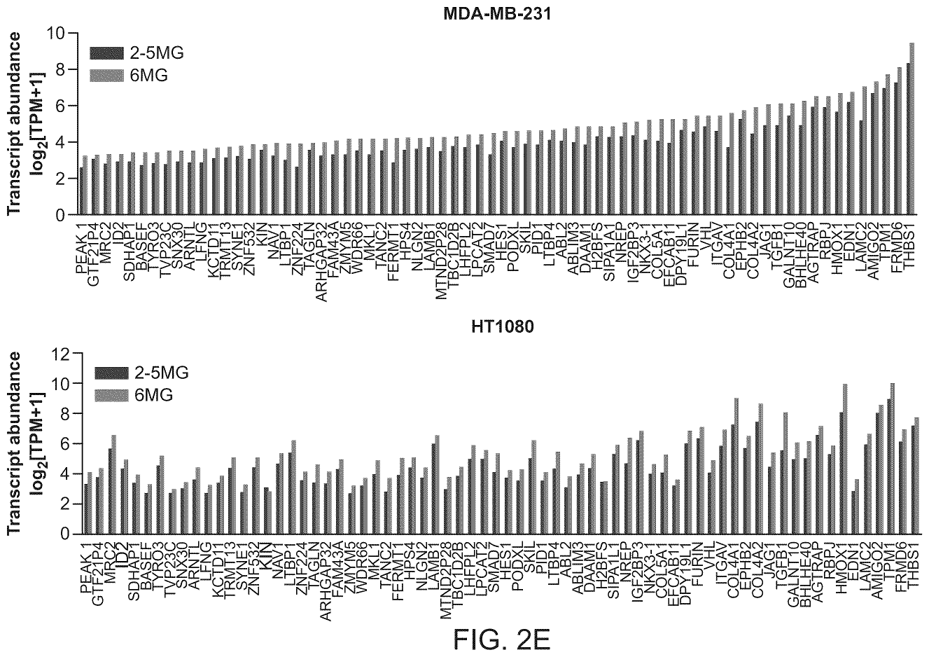

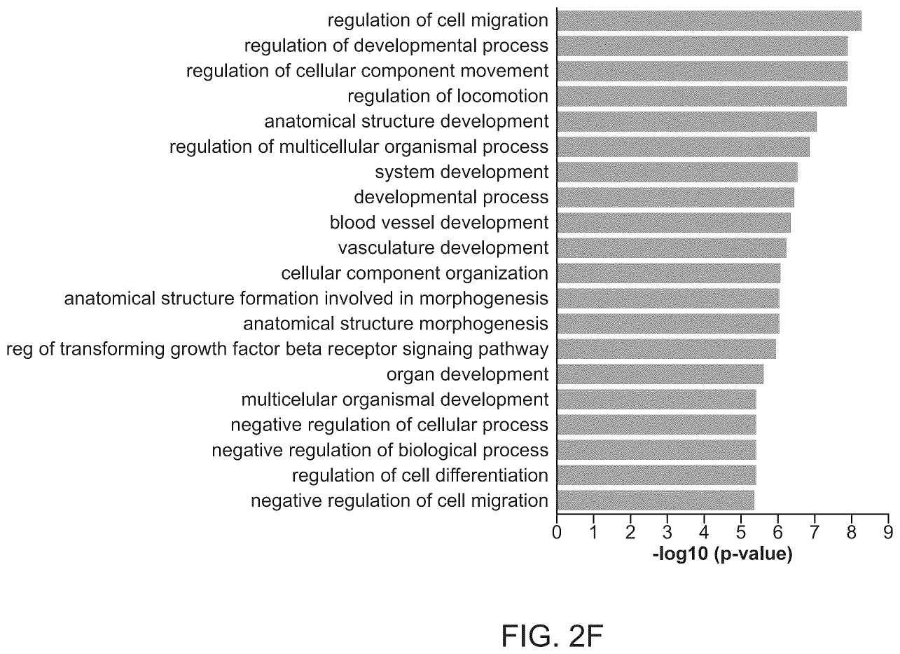

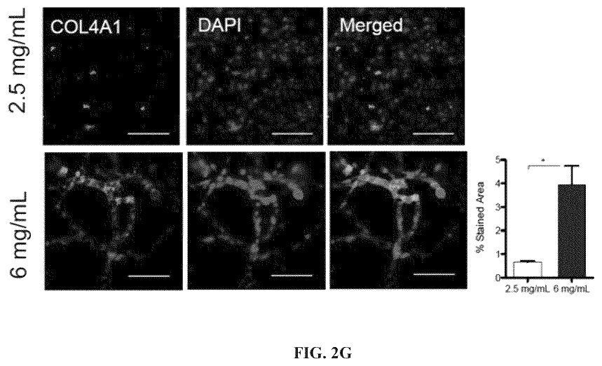

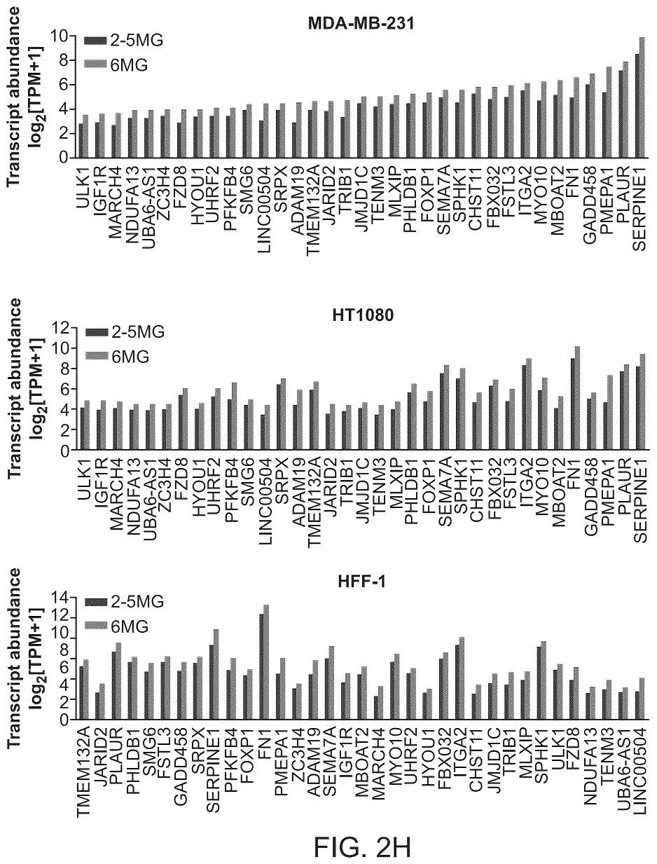

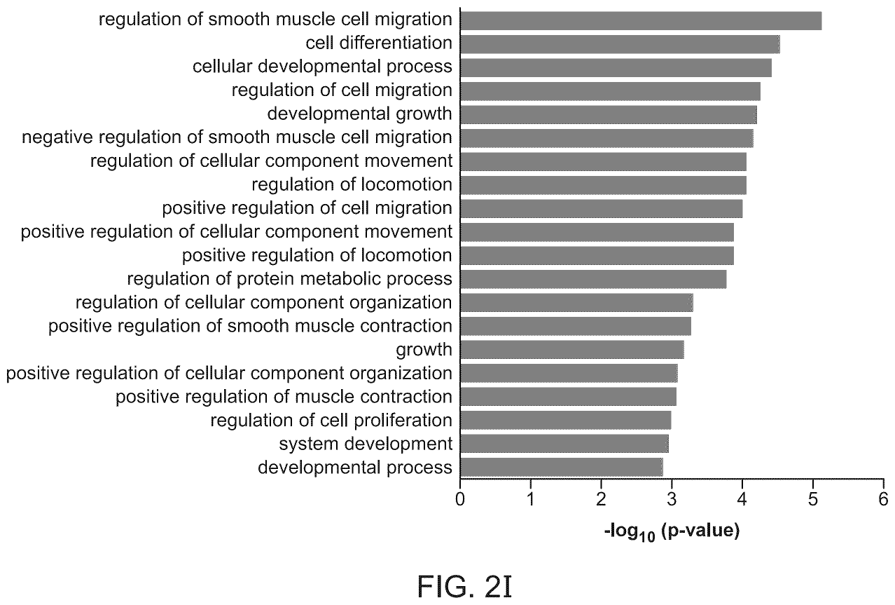

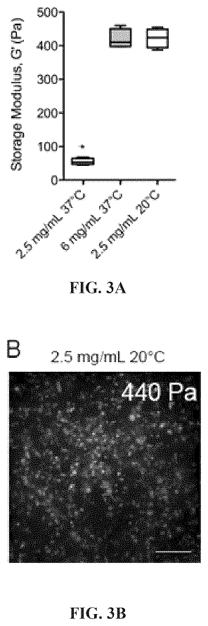

Accordingly, in some embodiments, the disclosed methods comprise determining or predicting a patient's prognosis (e.g., 5-year survival) or likelihood of metastasis by detect the expression levels of at least a subset of genes of interest in the disclosed VM module. Increased expression of at least subset of these genes is indicative of a decreased chance of survival, an increased likelihood of metastasis, and overall aggressive disease. Up-regulation or increased expression of the genes in the gene module can be relative to a defined control level. The control level may be determined by detecting expression levels of the genes in a non-cancerous sample from the patient or based on expression data in the general population. The subset of genes may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 of the genes in the disclosed VM module (see FIG. 2E), so long as the number of genes is sufficient to be predictive of prognosis in the patient.

While the methods may be used to determine prognosis or risk of metastasis of all subject or cancer patients, the disclosed methods are particularly useful for predicting prognosis or risk of metastasis of patient with stage I or stage II cancers. Average accuracy for VM phenotype prediction need not be 100% in order to provide clinical benefit. For instance, subtype prediction may be 80, 81, 82, 83, 84, 85, 86, 87, 88, 89, 90, 91, 92, 93, 94, 95, 96, 97, 98, 99, or 100% accurate.

Moreover, the disclosed methods may also comprise determining the pore size of the collagen in a tumor and the expression level of β1 integrin, as a small pore size and increased β1 integrin expression are also indicative of poor prognosis.

Furthermore, the disclosed methods are applicable to all types of cancer. In particular, the disclosed methods are predictive of patient prognosis in patients with breast cancer, glioma, cervical squamous cell carcinoma, endocervical adenocarcinoma, lung adenocarcinoma, kidney renal clear cell carcinoma, and pancreatic adenocarcinoma.

In some embodiments, the disclosed methods comprise determining the VM phenotype of a cell by detecting at least a subset of genes in the VM gene module. For instance, the subset may be about 30, 35, 40, 45, 50, 55, 60, 65, or 70 genes of interest, or a sufficient number of genes to predict the phenotype of the cell.

The disclosure further provides diagnostic, prognostic and therapeutic methods, which are based, at least in part, on determination of the expression of one or more genes of the VM module identified herein.

For example, information obtained using the diagnostic assays described herein is useful for determining if a subject is suitable for cancer treatment of a given type. Based on the prognostic information, a doctor can recommend a therapeutic protocol, useful for reducing the malignant mass or tumor in the patient or treat cancer in the individual.

A patient's likely clinical outcome can be expressed in relative terms. For example, a patient having a particular expression level can experience relatively shorter overall survival than a patient or patients not having the expression level. The patient having the particular expression level, alternatively, can be considered as likely to have poor prognosis. Similarly, a patient having a particular expression level can experience relatively shorter progression free survival, or time to tumor progression, than a patient or patients not having the expression level. The patient having the particular expression level, alternatively, can be considered as likely to suffer metastasis and/or tumor progression. Further, a patient having a particular expression level can experience relatively shorter time to tumor recurrence than a patient or patients not having the expression level. The patient having the particular expression level, alternatively, can be considered as likely to suffer tumor recurrence. Yet in another example, a patient or tumor sample having a particular expression level can experience relatively more complete response or partial response than a tumor, subject, patient or patients not having the expression level. The patient having the particular expression level, alternatively, can be considered as likely to respond.

It is to be understood that information obtained using the diagnostic assays described herein can be used alone or in combination with other information, such as, but not limited to, genotypes or expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes or expression levels of one or more genes as disclosed herein are used in a panel of genes, each of which contributes to the final diagnosis, prognosis or treatment.

The methods are useful in the assistance of an animal, a mammal or yet further a human patient. For the purpose of illustration only, a mammal includes but is not limited to a human, a simian, a murine, a bovine, an equine, a porcine or an ovine subject.

### Kits for Predicting Prognosis and Likelihood of Metastasis

In some embodiments, the disclosure provides for kits for amplifying and/or determining the expression of at least a portion of the VM biomarkers. The kits may comprise probes or primers capable of hybridizing to the genes of interest and instructions for use, while in some embodiments the kits may comprise an array comprising the genes of interest.

The kits comprise one of more of the compositions described above and instructions for use. A kit may comprise oligonucleotides for amplifying and/or detecting the genes of the VM module. Oligonucleotides “specific for” a gene of interest may bind either to the gene locus or bind adjacent to the gene locus. For oligonucleotides that are to be used as primers for amplification, primers are adjacent if they are sufficiently close to be used to produce a polynucleotide comprising the polymorphic region. In one embodiment, oligonucleotides are adjacent if they bind within about 1-2 kb, and preferably less than 1 kb from the gene of interest. Specific oligonucleotides are capable of hybridizing to a sequence, and under suitable conditions will not bind to a sequence differing by a single nucleotide.

In some embodiments, the kit can comprise at least one probe or primer which is capable of specifically hybridizing to the polymorphic region of the gene of interest and instructions for use. The kits preferably comprise at least one of the above described nucleic acids. Preferred kits for amplifying at least a portion of the genes of interest comprise two primers. Such kits are suitable for detection of the VM gene module by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the invention comprise at least one reagent necessary to perform the assay. For example, the kit can comprise an enzyme. Alternatively the kit can comprise a buffer or any other necessary reagent.

Conditions for incubating a nucleic acid probe with a test sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present invention. Examples of such assays can be found in Chard, T. (1986) “An Introduction to Radioimmunoassay and Related Techniques” Elsevier Science Publishers, Amsterdam, The Netherlands; Bullock, G. R. et al., “Techniques in Immunocytochemistry” Academic Press, Orlando, Fla. Vol. 1 (1982), Vol. 2 (1983), Vol. 3 (1985); Tijssen, P., (1985) “Practice and Theory of Immunoassays: Laboratory Techniques in Biochemistry and Molecular Biology”, Elsevier Science Publishers, Amsterdam, The Netherlands.

The test samples used in the diagnostic kits include cells, protein or membrane extracts of cells, or biological fluids such as sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region of the gene of interest.

As amenable, these suggested kit components may be packaged in a manner customary for use by those of skill in the art. For example, these suggested kit components may be provided in solution or as a liquid dispersion or the like.

This disclosure utilizes experimentally observed indicia of cancer metastasis including: high density collagen promotes persistent migration in cancer cells; increased in invasion persistence occurs after cell division in high density collagen, but not in low density; and post division polarization initiates migration consistent with tubular structure formation. Detection of any of these indicia may be incorporated into a kit or used in the disclosed methods.

### Culture in a 3D Collagen Matrix

In another aspect, provided herein is a method of determining the migration capacity of a tumor comprising tumor cells, the method comprising, consisting of, or consisting essentially of: culturing a tumor sample embedded in a 3D collagen matrix, wherein the tumor sample was isolated from a subject; and determining the migration capacity of the tumor sample by tracking motility of the tumor cells in the 3D collagen matrix. As used herein, a tumor sample embedded in a 3D matrix refers to a condition where the sample is fully embedded, in contact with matrix components on all sides, and located a sufficient distance away from the bottom and sides of the container (e.g. culture dish or coverslip bottom) to avoid their influence.

Collagen is a structural protein that is generally found in connective tissue and the extracellular space of animals. Collagen is classified into several types including but not limited to type I (e.g. COL1A1 (Entrez gene: 1277, UniProt: P02452); COL1A2 (Entrez gene: 1278, UniProt: P08123)), type II (e.g. COL2A1 (Entrez gene: 1280, UniProt: P02458)), type III (e.g. COL3A1 (Entrez gene: 1281, UniProt: P02461)), type IV (basement membrane collagen, e.g. COL4A1 (Entrez gene: 1282, UniProt: P02462), COL4A2 (Entrez gene: 1284, UniProt: P08572), COL4A3 (Entrez gene: 1285, UniProt: Q01955), COL4A4 (Entrez gene: 1286, UniProt: P53420), COL4A5 (Entrez gene: 1287, UniProt: P29400), COL4A6 (Entrez gene: 1288, UniProt: Q14031)), type V (e.g. COL5A1 (Entrez gene: 1289, UniProt: P20908), COL5A2 (Entrez gene: 1290, UniProt: P05997), COL5A3 (Entrez gene: 5059, UniProt: P25940)), type VI (e.g. COL6A1 (Entrez gene: 1291, UniProt: P12109), COL6A2 (Entrez gene: 1292, UniProt: P12110), COL6A3 (Entrez gene: 1293, UniProt: P12111), COL6A5 (Entrez gene: 256076, UniProt: PA8TX70, HOY935)), type VII (e.g. COL7A1 (Entrez gene: 1294, UniProt: Q02388)), type VIII (e.g. COL8A1 (Entrez gene: 1295, UniProt: P27658), COL8A2 (Entrez gene: 1296, UniProt: P25067, Q4VAQ0)), type IX (e.g. COL9A1 (Entrez gene: 1297, UniProt: P20908), COL9A2 (Entrez gene: 1290, UniProt: P05997), COL9A3 (Entrez gene: 5059, UniProt: P25940)), type X (e.g. COL10A1 (Entrez gene: 1300, UniProt: A03692)), type XI (e.g. COL11A1 (Entrez gene: 1301, UniProt: P12107), COL11A2 (Entrez gene: 1302, UniProt: P13942)), type XII (e.g. COL12A1 (Entrez gene: 1303, UniProt: Q99715)), or type XIII (e.g. COL10A1 (Entrez gene: 1300, UniProt: A03692). In particular embodiments, the collagen is type IV collagen. Collagens are available from, for example, Sigma Aldrich, St. Louis, Missouri, U.S.A. (e.g. CAS #9007-34-5, cat. #: C6745).

In some embodiments, the 3D collagen matrix comprises a high density of collagen. In some embodiments, the collagen density is selected from the group of: from about 4 mg/mL to about 10 mg/mL, from about 4 mg/mL to about 8 mg/mL, or from about 4 mg/mL to about 6 mg/mL. In a particular embodiment, the collagen density is about 6 mg/mL.

In some embodiments, the 3D collagen matrix comprises, consists of, or consists essentially of a median fiber length less than or equal to 9.5 μm, less than or equal to 9 μm, less than or equal to 8.5 μm, less than or equal to 8 μm, less than or equal to 7.5 μm, less than or equal to 7 μm less than or equal to 6.5 μm, less than or equal to 6 μm, less than or equal to 5.5 μm, less than or equal to 5 μm, or less than or equal to 4.5 μm.

In some embodiments, the 3D collagen matrix comprises, consists of, or consists essentially of a median pore size less than or equal to 10 μm, less than or equal to 9.5 μm, less than or equal to 9 μm, less than or equal to 8.5 μm, less than or equal to 8 μm, less than or equal to 7.5 μm, less than or equal to 7 μm less than or equal to 6.5 μm, less than or equal to 6 μm, less than or equal to 5.5 μm, less than or equal to 5 μm, less than or equal to 4.5 μm, less than or equal to 4 μm, less than or equal to 3.5 μm, or less than or equal to 3.5 μm.

In some embodiments, the 3D collagen matrix further comprises a molecular crowding agent. Nonlimiting examples include one or more of: polyethylene glycol (e.g., PEG1450, PEG3000, PEG8000, PEG10000, PEG14000, PEG15000, PEG20000, PEG250000, PEG30000, PEG35000, PEG40000, PEG compound with molecular weight between 15,000 and 20,000 daltons, or combinations thereof), polyvinyl alcohol, dextran and ficoll. In some embodiments, the crowding agent is present in the reaction mixture at a concentration between 1 to 12% by weight or by volume of the matrix, e.g., between any two concentration values selected from 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, and 12.0%. In a particular embodiment, the molecular crowding agent is polyethylene glycol (PEG).

In particular embodiments, the 3D collagen matrix comprises, consists of, or consists essentially of about 2 mg/mL to about 6 mg/mL collagen and at least 4 mg/mL PEG. In a particular embodiment, the 3D collagen matrix comprises 2.5 mg/mL collagen and 6 mg/mL PEG.

In some embodiments, motility is tracked by imaging the embedded tumor sample. The tumor sample may be imaged by any method known in the art including, but not limited to, microscopy, confocal microscopy, optical coherence tomography, multiphoton microscopy, time lapse microscopy, live microscopy, and video microscopy. Additional methods of imaging 3D cultures are described in Graf, B. and Boppart, S. Methods Mol. Biol. (2010) 591:211-27. In some embodiments, the embedded tumor sample is imaged at least once per day. In other embodiments, the embedded tumor sample is imaged at least once every two days. In other embodiments, the embedded tumor sample is imaged at least once every three days. In some embodiments, at least one image of the embedded tumor sample is analyzed to characterize tumor cell migration and/or motility. In some embodiments, the image is analyzed using an image processing algorithm.

In some embodiments, the method further comprises determining an invasion distance of a tumor cell, quantifying network structures formed by the tumor cells, determining the length of network structures formed by the tumor cells, and or/determining the shape of a tumor cell.

In some embodiments, the method further comprises determining a gene expression level of one or more genes of a VM gene module in the tumor sample as described herein.

In some embodiments, the tumor sample is a biopsy tissue sample or a circulating tumor cell liquid biopsy sample.

In another aspect, provided herein is a method of screening a tumor for sensitivity to a drug, the method comprising, consisting of, or consisting essentially of: culturing a tumor sample embedded in a 3D collagen matrix comprising one or more drugs; and screening the tumor sample for sensitivity to the drug by determining the viability of the tumor sample. The drug may comprise any known or suspected cancer therapeutic including but not limited to the cancer therapeutics described herein.

The concentration of the drug in the 3D collagen matrix ranges from about 1 mM to about 100 mM, about 1 mM to about 50 mM, about 1 mM to about 40 mM, about 1 mM to about 30 mM, about 1 mM to about 25 mM, about 1 mM to about 20 mM, about 1 mM to about 15 mM, about 1 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 2 mM, about 3 mM to about 50 mM, about 3 mM to about 30 mM, about 3 mM to about 25 mM, about 3 mM to about 20 mM, about 3 mM to about 15 mM, about 3 mM to about 10 mM, about 3 mM to about 9 mM, about 3 mM to about 8 mM, about 3 mM to about 7 mM, about 3 mM to about 6 mM, about 3 mM to about 5 mM, about 6 mM to about 50 mM, about 6 mM to about 30 mM, about 6 mM to about 25 mM, about 6 mM to about 15 mM, or about 6 mM to about 10 mM. Alternatively, the concentration of the drug ranges from about 10 μM from about 1 μM to about 100 μM, about 1 μM to about 50 μM, about 1 μM to about 40 μM, about 1 μM to about 30 μM, about 1 μM to about 25 μM, about 1 μM to about 20 μM, about 1 μM to about 15 μM, about 1 μM to about 10 μM, about 1 μM to about 9 μM, about 1 μM to about 8 μM, about 1 μM to about 7 μM, about 1 μM to about 6 μM, about 1 μM to about 5 μM, about 1 μM to about 2 μM, about 3 μM to about 50 μM, about 3 μM to about 30 μM, about 3 μM to about 25 μM, about 3 μM to about 20 μM, about 3 μM to about 15 μM, about 3 μM to about 10 UM, about 3 μM to about 9 μM, about 3 μM to about 8 μM, about 3 μM to about 7 μM, about 3 μM to about 6 μM, about 3μ M to about 5μ, about 6μ M to about 50μ, about 6μ M to about 30μ, about 6 μM to about 25 μM, about 6 μM to about 15 μM, or about 6 μM to about 10 μM. Alternatively, the concentration of the drug ranges from about 1 nM to about 100 nM, about 1 nM to about 50 nM, about 1 nM to about 40 nM, about 1 nM to about 30 nM, about 1 nM to about 25 nM, about 1 nM to about 20 nM, about 1 nM to about 15 nM, about 1 nM to about 10 nM, about 1 nM to about 9 nM, about 1 nM to about 8 nM, about 1 nM to about 7 nM, about 1 nM to about 6 nM, about 1 nM to about 5 nM, about 1 nM to about 2 nM, about 3 nM to about 50 nM, about 3 nM to about 30 nM, about 3 nM to about 25 nM, about 3 nM to about 20 nM, about about 3 nM to about 15 nM, about 3 nM to about 10 nM, about 3 nM to about 9 nM, about 3 nM to about 8 nM, about 3 nM to about 7 nM, about 3 nM to about 6 nM, about 3 nM to about 5 nM, about 6 nM to about 50 nM, about 6 nM to about 30 nM, about 6 nM to about 25 nM, about 6 nM to about 15 nM, or about 6 nM to about 10 nM.

Tumor viability may be detected by any method known in the art including but not limited to staining with trypan blue, staining with annexin, determining viability by light microscopy, refraction, and cell morphology, flow cytometry, dye uptake, and commercially available viability kits such as the LIVE/DEAD™ Viability/Cytotoxicity Kit for mammalian cells (available from Thermo Fisher Scientific, Cat #L3224).

In another aspect, provided herein is a culture system comprising, consisting of, or consisting essentially of cells embedded in a high density 3D collagen matrix. In some embodiments, the 3D collagen matrix comprises a high density of collagen. In some embodiments, the collagen density is selected from the group of: from about 4 mg/mL to about 10 mg/mL, from about 4 mg/mL to about 8 mg/mL, or from about 4 mg/mL to about 6 mg/mL. In a particular embodiment, the collagen density is about 6 mg/mL.

In some embodiments, the 3D collagen matrix comprises, consists of, or consists essentially of a median fiber length less than or equal to 9.5 μm, less than or equal to 9 μm, less than or equal to 8.5 μm, less than or equal to 8 μm, less than or equal to 7.5 μm, less than or equal to 7 μm less than or equal to 6.5 μm, less than or equal to 6 μm, less than or equal to 5.5 μm, less than or equal to 5 μm, or less than or equal to 4.5 μm.

In some embodiments, the 3D collagen matrix comprises, consists of, or consists essentially of a median pore size less than or equal to 10 μm, less than or equal to 9.5 μm, less than or equal to 9 μm, less than or equal to 8.5 μm, less than or equal to 8 μm, less than or equal to 7.5 μm, less than or equal to 7 μm less than or equal to 6.5 μm, less than or equal to 6 μm, less than or equal to 5.5 μm, less than or equal to 5 μm, less than or equal to 4.5 μm, less than or equal to 4 μm, less than or equal to 3.5 μm, or less than or equal to 3.5 μm.

In some embodiments, the 3D collagen matrix further comprises a molecular crowding agent. Nonlimiting examples include one or more of: polyethylene glycol (e.g., PEG1450, PEG3000, PEG8000, PEG10000, PEG14000, PEG15000, PEG20000, PEG250000, PEG30000, PEG35000, PEG40000, PEG compound with molecular weight between 15,000 and 20,000 daltons, or combinations thereof), polyvinyl alcohol, dextran and ficoll. In some embodiments, the crowding agent is present in the reaction mixture at a concentration between 1 to 12% by weight or by volume of the matrix, e.g., between any two concentration values selected from 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, and 12.0%. In a particular embodiment, the molecular crowding agent is polyethylene glycol (PEG).

### EQUIVALENTS

One skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims.

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

The disclosure illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of that claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

Other features and advantages of will be apparent to those of skill in the art from the following examples and claims. For instance, commercial applications of the disclosed methods and kits include a personalized medicine diagnostic tool for cancer patients, which takes into account the molecular makeup of the tumor and can help differentiate aggressive from indolent disease.

Particular embodiments of the disclosure further described by reference to the following examples, which are provided for illustration only. The present disclosure is not limited to the examples, but rather includes all variations that are evident from the teachings provided herein.

### EXAMPLES

### Example 1—3D Collagen Architecture Induces Vascular Mimicry

The tumor microenvironment is heterogeneous from both a cellular and an extracellular matrix (ECM) perspective. Regions of dense, stiff, or aligned collagen fibers have each been implicated in locally driving aggressive tumor cell migration behaviors that are thought to contribute to metastatic progression. Cell-to-cell differences in innate migration and metastatic capabilities have also been described. However, it remains unclear how intrinsic tumor cell factors and extrinsic ECM factors work together to promote the emergence of distinct migration phenotypes, and whether some migration phenotypes contribute more to metastasis than others. To probe the extrinsic basis of cancer cell migration regulation, MDA-MB-231 breast cancer and HT1080 fibrosarcoma cells were embedded within engineered 3D collagen matrices of varying architectures and used high throughput time-lapse microscopy to monitor single cancer cell migration. A collagen matrix architecture defined by small pores and short fibers was identified that gives rise to two subpopulations of breast cancer cells wherein migration is differentially regulated. In this matrix architecture, the majority of cells adopted a rapid, persistent migration behavior while the minority population migrated slowly and randomly. After seven days, rapidly migrating cells organized into long interconnected networks coated with basement membrane, a phenotype known as vascular mimicry (VM). In contrast, cells undergoing slow migration formed spheroids. The network-forming versus spheroid-forming migration response was not mediated by hypoxia or matrix stiffness, but rather matrix architecture and β1 integrin expression. Fibrosarcoma cells also displayed the network-forming phenotype. In both breast and fibrosarcoma cells, this phenotype was associated with the upregulation of a conserved transcriptional program enriched for genes involved in vascular development and regulation of cell migration. This gene module was predictive of poor survival in multiple human tumor transcriptome datasets. Thus, the engineered 3D collagen model system revealed that VM arises from a cancer cell-intrinsic transcriptional and migratory response triggered by 3D collagen architecture through integrin β1 and represents a unique system for studying the migration behavior underlying VM. Furthermore, these analyses suggest that matrix-induced VM migration may be broadly relevant as a driver of metastatic progression in solid human cancers.

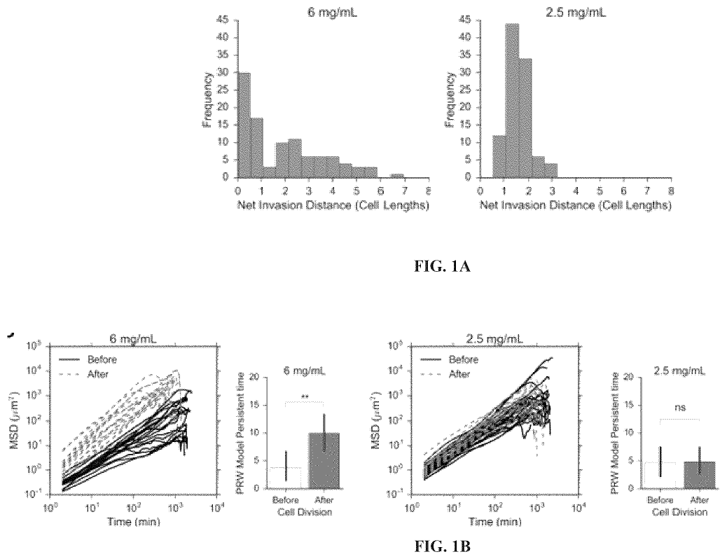

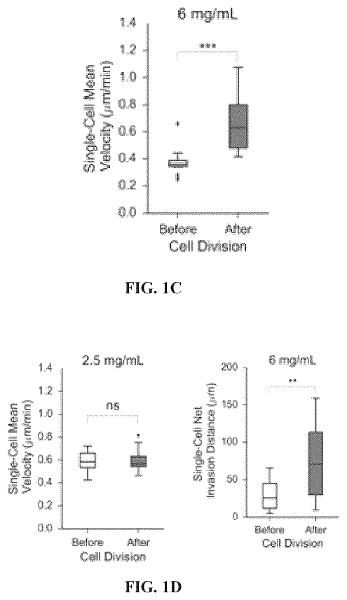

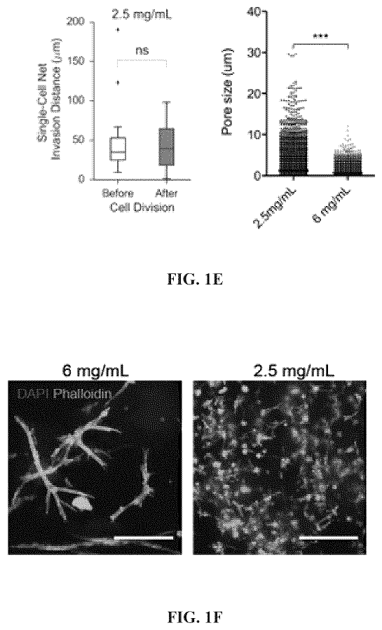

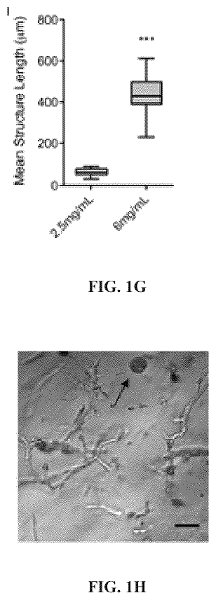

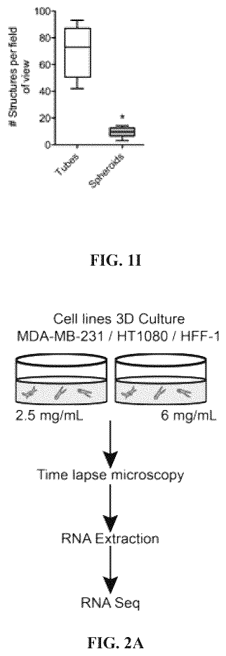

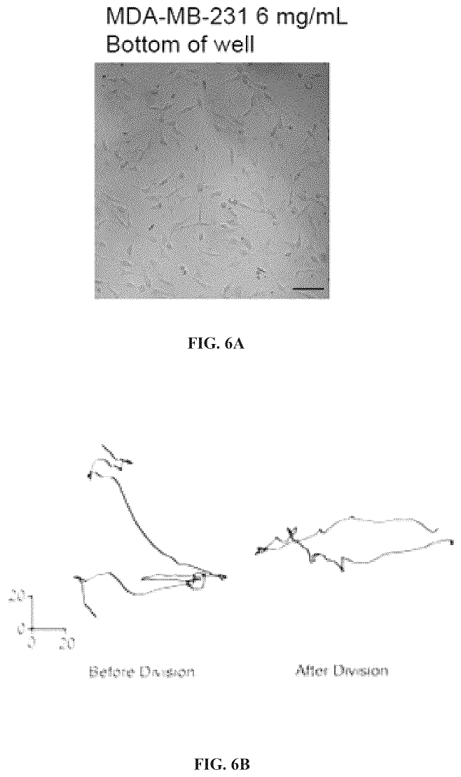

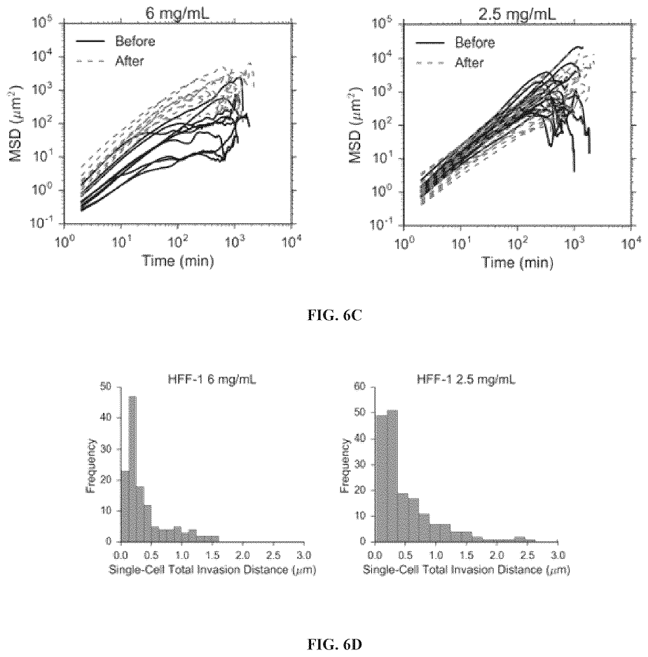

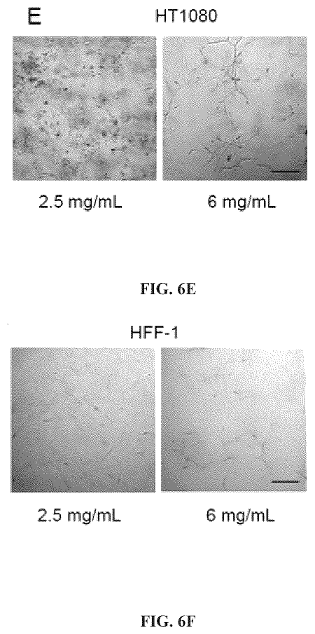

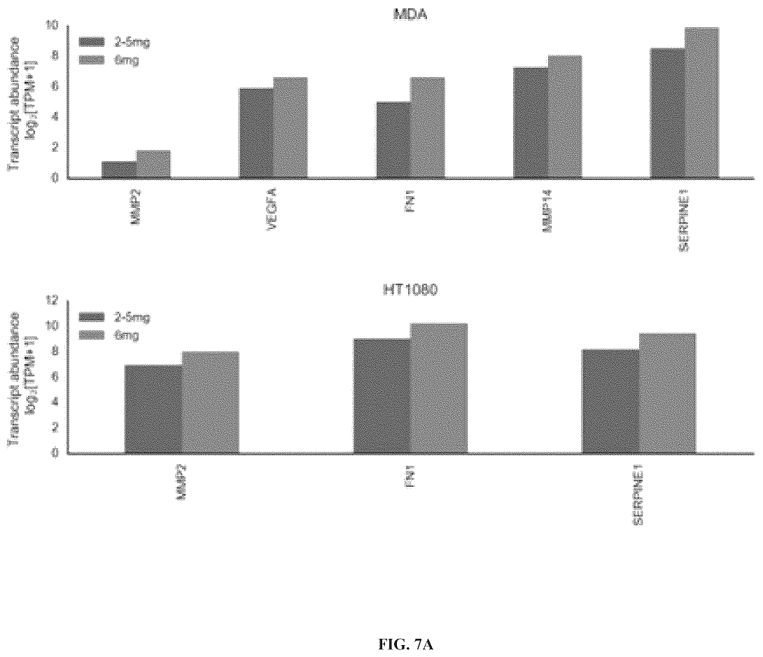

To investigate the role of the 3D collagen microenvironment on the migration phenotype of breast cancer cells, MDA-MB-231 cells were embedded in collagen I matrices of varying densities mimicking normal breast tissue, 2.5 mg/mL collagen, and cancerous breast tissue, 6 mg/mL collagen. Long-term time-lapse microscopy was used to monitor the migration response of single cells in these conditions. Analysis of the invasion distance of individual cells revealed that cells embedded in the high density environment displayed two distinct phenotypes. Some of the cells moved less than 1 cell length (characteristic cell length taken as 50 μm) from their initial position while the remaining cells invaded to distances up to 7 cell lengths over the course of 48 hrs (FIG. 1A, left). On the other hand, cells in lower density environments behaved homogeneously and invaded to distances less than 3 cell lengths during the observation period (FIG. 1A, right). Cells migrating in dense collagen initially appeared to be trapped and were unable to invade. However, after one division cycle, most cells switched to a highly invasive motility behavior, significantly increasing their persistence, velocity, and total invasion distance (FIG. 1, B-D, left panels). This behavior was not observed in cells embedded in the low density matrix, where cell migration was the same before and after division (FIG. 1, B-D, right panels). Interestingly, cells embedded in the high density condition but in contact with the coverslip (FIG. 6A) did not undergo the same migration transition upon division (FIG. 6B).

Without being bound by theory, ECM structural heterogeneity could be responsible for the observed migration heterogeneity in high density but not low density collagen. To assess structural heterogeneity, matrix pore sizes were measured in each condition by analysis of confocal reflection imaging of collagen fibers. Interestingly, pore size distributions in the high density matrix were more homogeneous than in the low density matrix (FIG. 1E). The coefficient of variation (CV) of pore size was 96% and 176% respectively. Yet, migration behavior was more heterogeneous in the high density matrix (CV=86%) than in the low density (CV=29%) (FIG. 1A). This suggested that the two distinct migration phenotypes that arose in dense matrix conditions were not a result of a non-homogeneous matrix environment, but instead stemmed from intrinsically different responses to the matrix environment.

The motility responses observed in 2.5 and 6 mg/mL collagen matrices were not unique to MDA-MB-231 breast cancer cells. Similar migration patterns were observed for HT-1080 fibrosarcoma cells embedded in the same collagen matrix conditions (FIG. 6C), suggesting that these responses may be shared among distinct cancer cell types. To further examine whether the observed mesenchymal migration behavior was cell type dependent, the response of normal mesenchymal human foreskin fibroblasts (HFF-1) to low and high density collagen conditions was tested. Over an observation period of 48 hrs HFF cell migration was homogeneous with very low persistence. Cells invaded less than three cell lengths in low density collagen. In high density, HFFs elongated but did not invade more than one and a half cell lengths (FIG. 6D).

That both MDA-MB-231 and HT-1080 cancer cells migrated faster and further in high density collagen conditions was unexpected. Intuitively, cell migration would be expected to slow in dense conditions where more matrix must be remodeled to enable cell movement. Moreover, this behavior was common to both cancer cell types but not displayed by normal fibroblasts, which represent residents of the tumor stroma and also undergo mesenchymal migration in collagen. The long-term implications of the rapid migration phenotype induced in cancer cells under high density conditions was investigated. After one week of culture in high density collagen, breast cancer cells undergoing rapid and persistent migration formed branched network structures that resembled the early stages of endothelial tubulogenesis (FIG. 1F, left). The average length of cellular networks after one week was 437 μm (FIG. 1G). However, the small fraction of breast cancer cells undergoing slow and random migration in high density collagen did not participate in network formation and instead formed spheroids (14%, FIGS. 1, H and I). In contrast, cells cultured in low density collagen for one week migrated slowly with low persistence, and remained as single cells (FIG. 1F, right). The transition from single cell migration to network formation is reminiscent of cells undergoing mesenchymal-to-endothelial transdifferentiation (MEndoT) whereas the transition from single cell migration to spheroid formation is reminiscent of cells undergoing mesenchymal-to-epithelial transdifferentiation (MEpiT). HT-1080 cells also formed branched network structures in high density collagen, but no subpopulation of spheroid-forming cells was evident (FIG. 6E). A lack of spheroid formation may be a result of their mesenchymal origin, whereas MDA-MB-231 cells are of epithelial origin. HT-1080s also remained as single cells in low density collagen (FIG. 6E). However, HFFs remained as single cells in both high and low density conditions (FIG. 6F). In low density collagen, HFFs invaded the gel homogeneously, whereas cells in high density collagen remained in place, but extended protrusions and elongated to reach cell lengths up to 300 μm.

Without being bound by theory, the persistent migration phenotype of cancer cells in high density conditions leading to network formation could be the result of a cancer-specific transcriptional response that activates unique cell motility pathways. To test this, RNA sequencing was conducted on MDA-MB-231, HT-1080, and HFF cells cultured in low and high density collagen matrices after 24 hours, the time point where most cancer cells in the high density collagen matrix had undergone at least one cycle of cell division and had begun to invade with increase persistence (FIG. 2A). Despite the two distinct phenotypes present in cancer cells cultured under high density conditions, their bulk transcriptional profile was expected to be dominated by the large majority phenotype, which were network-forming cells (86% of structures) 20. The data was analyzed to determine if genes were differentially regulated from low to high density collagen in each cell type and whether these genes represented unique or conserved transcriptional response modules. As expected, cell type accounted for the most variance in gene expression (FIG. 2B). However, after a z-score transformation of the gene expression of each cell type, the collagen matrix condition accounted for the bulk of the remaining variance in gene expression (FIG. 2C). This supported the presence of gene expression programs linked to collagen matrix conditions. Using a Venn Diagram approach to identify conserved expression modules, a set of 70 genes was generated that were significantly upregulated by both cancer cell types in response to high density collagen by more than 50% (TPM Fold change >=1.5) (FIGS. 2, D and E). Gene set enrichment analysis revealed that the 70 common-to-cancer genes were significantly enriched for annotations in blood vessel development and regulation of migration (FIG. 2F). Key genes involved in Notch signaling, i.e. RBPJ and LFNG, were among these. Importantly, JAG1, COL4A2, and THBS1 genes identified in this common-to-cancer gene set have been previously associated with a VM phenotype intrinsically displayed by metastatic melanoma cells21. Staining for COL4A2, demonstrated that cancer-cell networks were positive for this basement membrane protein (FIG. 2G). Without being bound by theory, the 70 genes module may represent a conserved signature for cancer cells that have transdifferentiated into a VM phenotype.

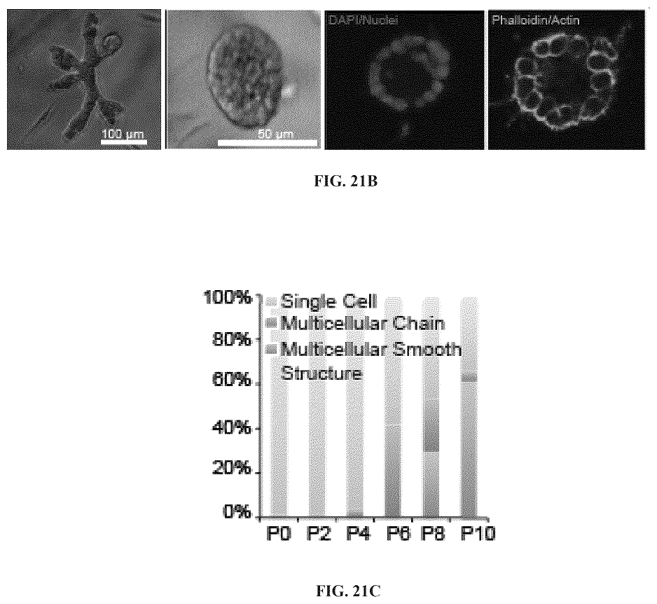

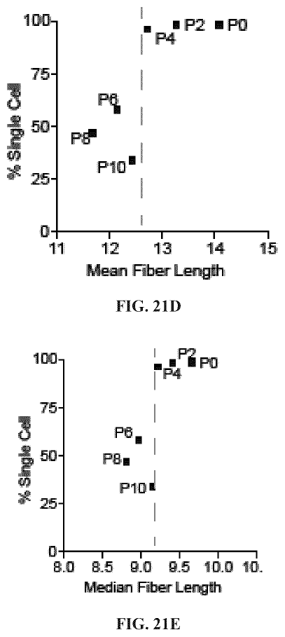

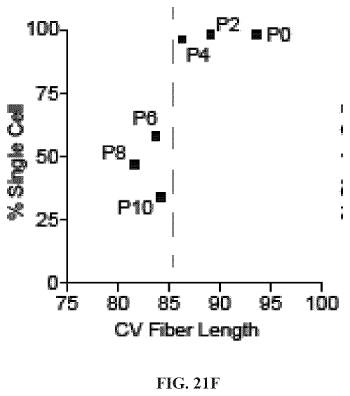

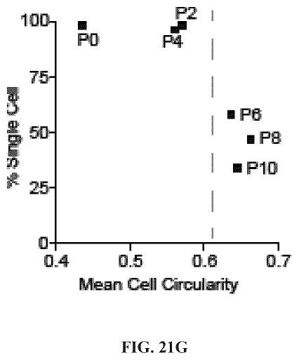

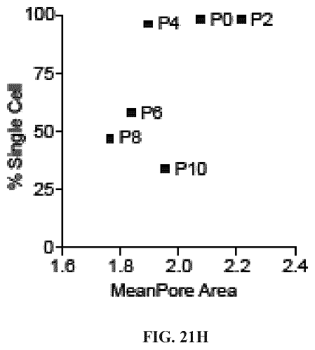

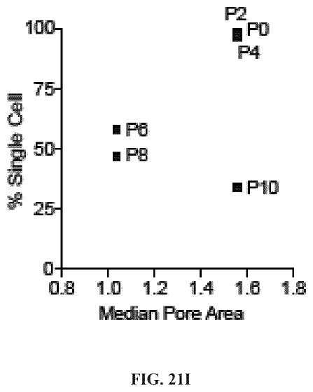

Further exploration of the datasets for individual cancer cell types revealed that, while some aspects of the VM transcriptional response were conserved, high density collagen also triggered the expression of genes related to vasculogenesis in a cell type dependent manner. For example, several additional genes previously implicated in VM were upregulated in breast cancer cells only (e.g. VEGFA Fold change=1.65, MMP2 Fold change=2.24), not in fibrosarcoma cells undergoing VM. A full list is shown. Interestingly, thirty-five genes were upregulated in response to high density collagen by all three cell types (FIG. 2H). These genes were enriched for annotations in cell differentiation and smooth muscle cell migration (FIG. 21). SERPINE1, a secreted protease inhibitor involved in coagulation and inflammation regulation, was identified in this common-to-all gene module. Several Serpine family members have previously been implicated as drivers of metastasis correlating with vascular mimicry and with brain metastases of lung and breast cancers. The finding that fibroblasts and cancer cells both upregulate SERPINE1 expression in high density ECM conditions hints at a potential supporting role for stromal cells in VM-mediated metastasis.

In human tumor biopsies, vessel-like structures that stain positively for basement membrane molecules but not for the endothelial marker CD31 have been associated with the phenomenon of VM. Without being bound by theory, it is believed that the high density 3D culture condition induces network-forming cells to undergo a form of MEndoT and that the 70 common-to-cancer genes identified were a signature of VM. Moreover, this transdifferentiation is induced by a feature of the high density 3D collagen culture condition that differed from the low density culture condition. Next, the matrix feature triggering transdifferentiation was identified including the physical parameters of stiffness, pore size, and fiber organization which differ between the 2.5 and 6 mg/mL collagen matrices. Without being bound by theory, chemical cues may also change. For example, adhesive ligand density and binding site-presentation to integrins and other matrix receptors may differ. Each of these features could potentially impact cancer cell motility behavior and gene expression.

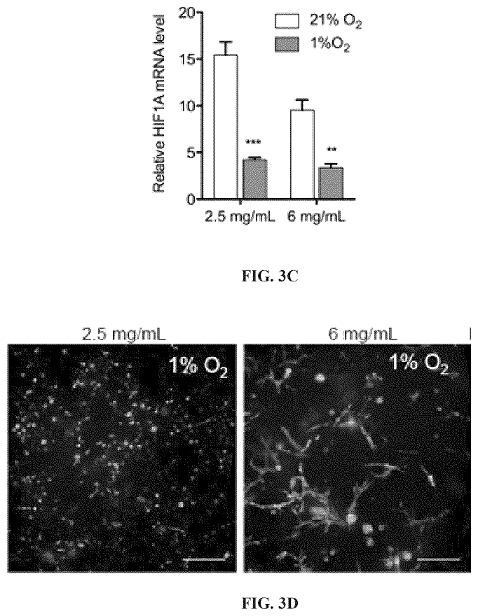

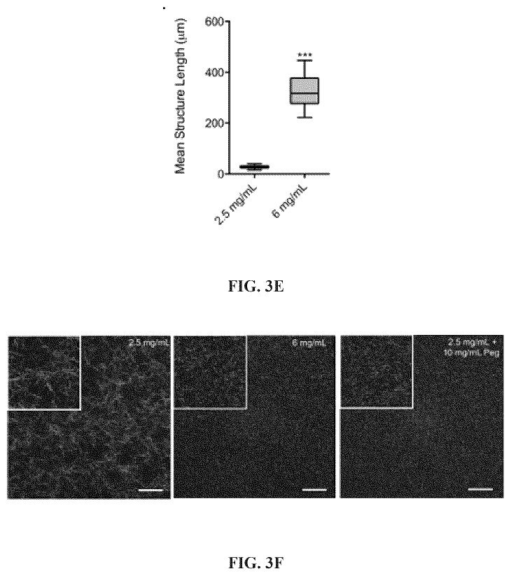

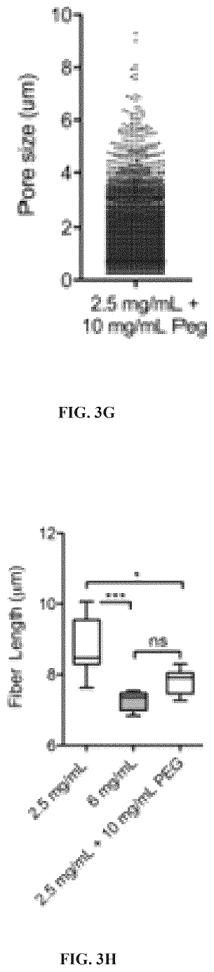

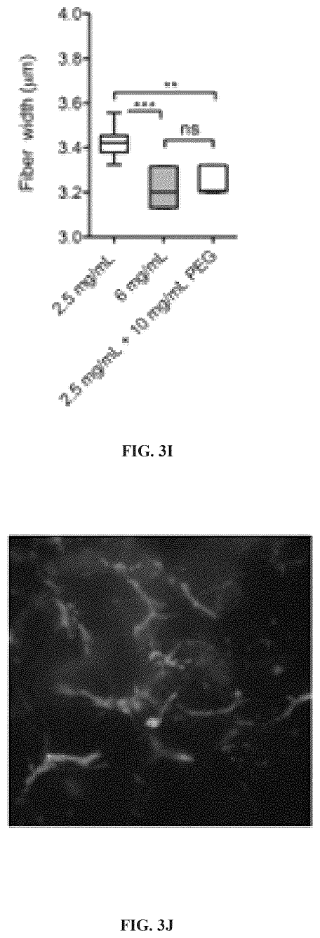

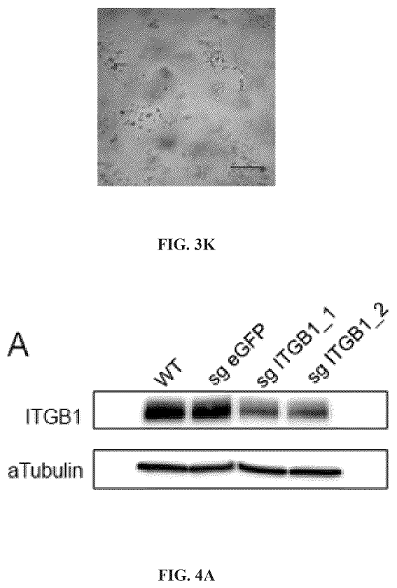

To determine whether increased stiffness of the high density collagen matrix14 was responsible for triggering transdifferentiation, a collagen polymerization procedure was developed that enhanced the stiffness of the low density matrix to match the stiffness of the high density matrix (FIG. 3A). By lowering the polymerization temperature from 37 °C to 20 °C, polymerization slowed, allowing fibers to form more organized and reinforced structures. Breast cancer cells cultured in stiffened low density conditions did not undergo network-forming VM or spheroid formation (FIG. 3B) suggesting that stiffness alone is not sufficient for triggering VM.

Next it was determined whether the smaller pore size of the high density matrices triggered transdifferentiation. Without being bound by theory, one way in which smaller pore sizes could influence cell behavior is by restricting the diffusion of molecules to and from the cells, including oxygen. Since regions of VM have previously been associated with markers of hypoxia in vivo, without being bound by theory, it is believed that high density collagen created a more hypoxic condition than low density collagen and that a lack of oxygen triggered susceptible cells to undergo VM. To test this, MDA-MB-231 cells were cultured in low density collagen under hypoxic conditions of 1% oxygen for one week. To confirm that a hypoxic response was achieved, the level of HIF1A mRNA expression was assessed by RT-qPCR. It was found that 7-day culture caused a significant decrease in HIF1A expression (FIG. 3C), a common response to long-term hypoxia by various cancer cell lines. Hypoxia was not sufficient to induce VM or spheroid formation in any portion of the cancer cell population in the low density collagen matrix (FIG. 3D, left). For comparison, the HIF1A mRNA expression of breast cancer cells cultured for one week in low density collagen under 21% oxygen, in high density collagen under 1% oxygen, and in high density collagen under 21% oxygen was also assessed (FIG. 3C). Without being bound by theory, these results suggested that cells cultured in high density collagen experience increased hypoxia compared to cells cultured in low density collagen under normal conditions. Nevertheless, the hypoxic response achieved in low density collagen under 1% oxygen exceeded that induced by high density matrix alone. Cells in high density matrix under 1% oxygen continued to predominately display a VM phenotype (FIG. 3D, right), but the average network length (FIG. 3E) was significantly shorter than cells in high density collagen under normoxic conditions (FIG. 1G, Wilcox signed rank text, p=6×10⁻⁴). Previous studies have also reported that hypoxia is not sufficient for induction of VM phenotype in melanoma cells in vitro. Without being bound by theory, it is possible that in vivo, additional stromal cell secreted factors or cell-cell interactions modulated by hypoxia may indirectly influence the VM process.

To further explore whether pore size reduction induced transdifferentiation of cancer cells, this parameter was interrogated independently of collagen density. In this model, the high density condition contains 2.4 times more collagen than the low density condition. This increase in total collagen reduces pore size, but also presents more adhesive ligands to cells, which could increase integrin activation. To separate pore size from bulk density, a collagen structure engineering technique was developed that reduced the pore size and fiber length of the low density matrix to approximate that of the high density matrix. Under normal polymerization conditions, low density collagen self-assembles into relatively long, structured fibers. When non-functionalized, inert polyethylene glycol (PEG) was mixed into collagen monomer solution prior to polymerization, molecular crowding restricted fiber formation. This resulted in shorter, more interconnected fibers yielding smaller pores (FIG. 3, F-I). Breast cancer cells encapsulated in this pore-size-reduced low density matrix underwent VM and spheroid formation over the course of one week (FIG. 3J). To control for the possible influence of PEG itself, PEG was added into media on top of a normally polymerized low density gel embedded with cells and allowed to diffuse into the interstitial spaces among the fibers to reach the same final concentration as was used in the pore-size-reduced low density matrix (10 mg/mL PEG). Cells maintained in this molecularly crowded condition over one week did not form networks or spheroids, but instead remained as single cells (FIG. 3K). However, a noticeable slowing of cell migration occurred, which resulted in an anisotropic patterning of single cells throughout the matrix. These results suggested that the matrix architecture of high density collagen induces VM independently of the bulk increase in adhesive ligand density.

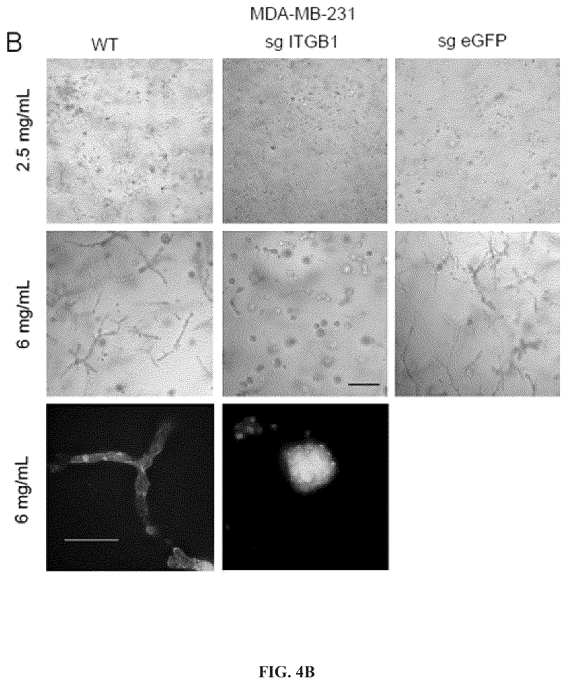

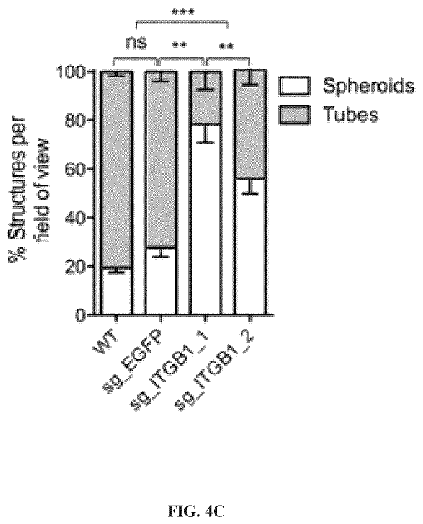

Confinement of cells in matrices with small pores could trigger VM transdifferentiation by limiting diffusion and thereby increasing autocrine signaling events. Short, homogeneously spaced fibers could also alter local collagen-cell interactions. Since β1 integrin (ITGB1) is a canonical receptor for collagen I and a central node in the ECM signal transduction pathway, without being bound by theory it was hypothesized that the expression level mediated the cellular response to confining collagen matrices. CRISPR-Cas9 technology was used to silence ITGB1 expression with single guide RNAs (sgRNAs) (sg_ITGB1, FIG. 4A) and silenced cells were again embedded sparsely in both low and high density collagen matrices. As a control, cells were transduced with CRISPR constructs expressing control sgRNAs targeting eGFP (FIG. 4A). After one week of culture, control cells exhibited the same behavior as the wild type in both collagen conditions (FIG. 4B). In low density collagen WT cells and sg_ITGB1 also behaved similarly remained as single cells after 1 week of culture. In the high density matrices WT cells formed VM structures and spheroids but cells with reduced β1 integrin expression (sg_ITGB1) formed significantly more spheroids than VM networks (FIG. 4B-C). This result indicated that β1 integrin expression regulates the fate of the cellular phenotype in high density collagen matrices. Cells with reduced β1 expression undergo transdifferentiation to an epithelial phenotype, whereas cells with increased expression transdifferentiate towards a VM phenotype. Further, this suggests that confinement in collagen may act first through diffusion limitations to induce multipotency, and second through a balance between cell-cell contact versus cell-matrix contact to mediate subsequent gene expression modules and transdifferentiation pathways. It is thought that aggressive cancer cells sustain pluripotency through aberrant expression of stem cell associated factors. Diffusion limitations may act to locally concentrate these factors thereby enhancing their autocrine activity.

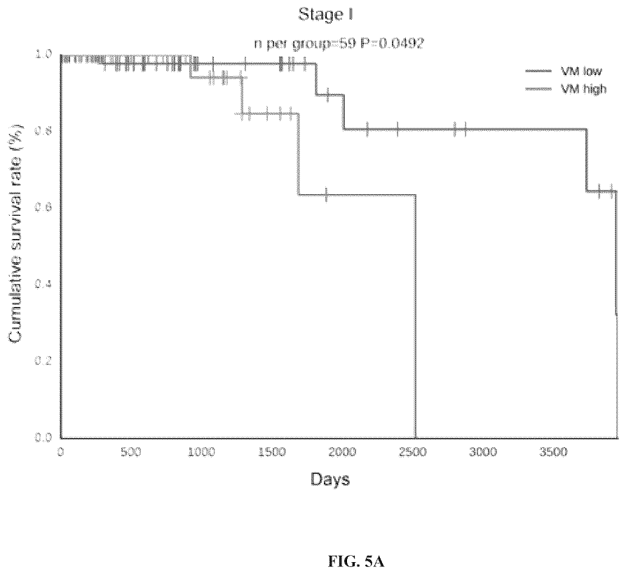

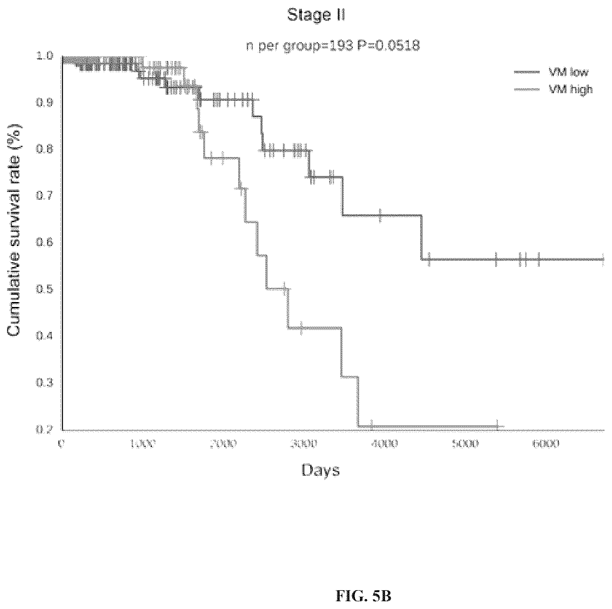

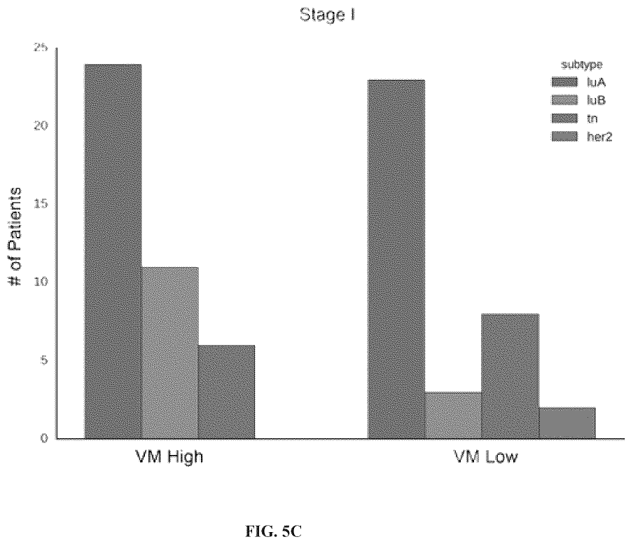

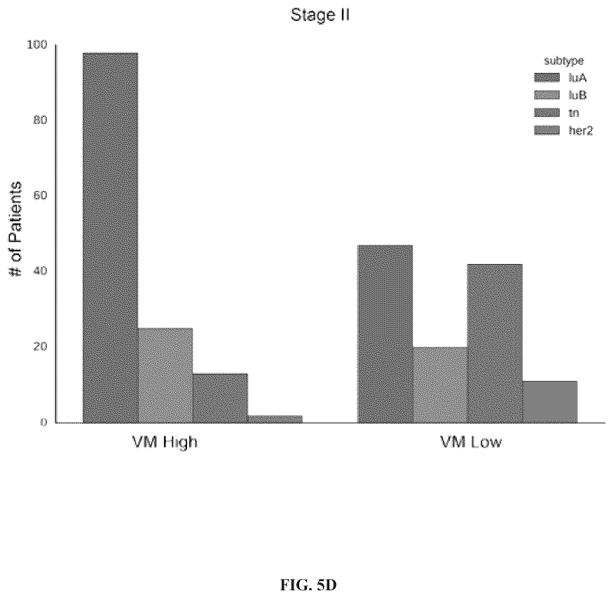

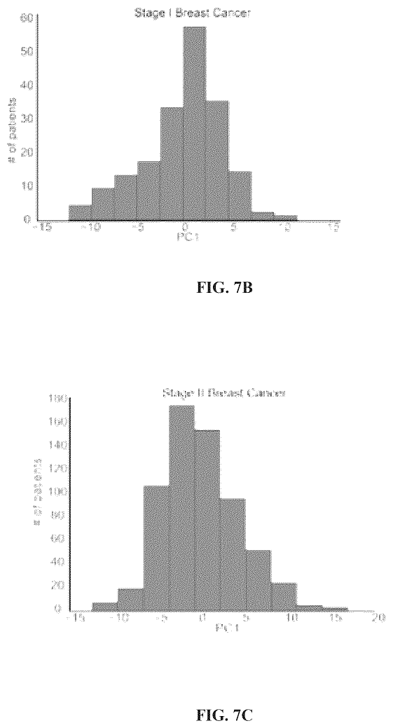

To determine if the VM transdifferentiation triggered by the 3D collagen system was clinically relevant, the 70 common-to-cancer genes associated with VM in vitro were assayed to determine if they could predict cancer patient prognosis. Without being bound by theory, it was anticipated that if this gene signature is indicative of a more metastatic cancer cell migration phenotype, its expression would correlate with poor patient outcomes. Since late stage tumors are already characterized by migration of tumor cells to distant lymph nodes or organs, a VM associated gene signature would correlate with prognosis in early (Stage I & II) but not late (Stage III & IV) stage tumors. Using the cancer genome atlas (TCGA), data was first analyzed for breast cancer patients with respect to the expression of the VM signature. An expression metagene was constructed using the loadings of the first principal component (PC1) of a 195 Stage I patient by 70 gene matrix (VM PC1) (FIG. 7B, also see Example 2-Methods). Then a survival analysis was conducted, comparing patients with the highest (top 30%) and lowest (bottom 30%) expression metagene scores by log rank test. The cumulative survival rate of these two groups differed significantly (p=0.05, FIG. 5A). Applying the same analysis to Stage II breast cancer patients (FIG. 5B and FIG. 7C) also revealed a significant difference in 5-year survival (p=0.05), indicating that the VM associated gene module could have clinical predictive power in early stage disease. In contrast, the VM module did not separate patients with better prognosis in late stage tumors (FIG. 7D). Importantly, in Stage I & II patients with shorter survival (top 30% in VM metagene expression), the frequency of breast cancer subtype was similar to the population background frequency of subtypes (FIGS. 5, C and D). This supported the theory that the VM expression metagene is predictive of 5-year survival independent of the molecular subtype of breast cancer. Finally, the predictive value of the VM gene module in additional cancer types analyzed by TCGA was examined. The VM gene module was a significant predictor of survival in lower grade glioma (p=2×10⁻⁸), cervical squamous cell carcinoma and endocervical adenocarcinoma (p=8×10⁻⁴), lung adenocarcinoma (p=0.0065), kidney renal clear cell carcinoma (p=0.0378), and pancreatic adenocarcinoma (p=0.0384).

This example describes a 3D in vitro model system designed to probe the physical basis of cancer cell migration responses to collagen matrix organization. Using this system, it was discovered that confining matrix architectures induced two distinct migration behaviors in breast cancer cells leading to spheroid formation or VM network formation. This the first identified physical driver of VM induction. ITGB1 modulated these migration responses and subsequent superstructure formation. Moreover, VM network formation was associated with a conserved transcriptional response used by multiple cancer cell types and that was predictive of patient survival in six clinical tumor datasets. These are the first identified core molecular markers of VM. Thus, without being bound by theory, these findings link a matrix-induced 3D migration phenotype and gene expression program to a clinical tumor cell phenotype driving blood borne metastasis.

### Example 2—Methods

Cell culture. HT-1080 and HFF-1 were purchased from (ATCC, Manassas, VA) MDA-MB-231 cells were provided by Adam Engler (UCSD Bioengineering). All cell lines were cultured in high glucose Dulbecco's modified Eagle's medium supplemented with 10% (v/v) fetal bovine serum (FBS, Corning, Corning, NY) and 0.1% gentamicin (Gibco Thermofisher, Waltham, MA) and maintained at 37 °C and 5% CO₂ in a humidified environment during culture and imaging. The cells were passaged every 2-3 days. Cell culture under hypoxia was done on a humidified and temperature controlled environment at 1% O₂.

3D culture in collagen I matrix. Cells embedded in 3D collagen matrices were prepared by mixing cells suspended in culture medium and 10× reconstitution buffer, 1:1 (v/v), with soluble rat tail type I collagen in acetic acid (Corning, Corning, NY) to achieve the desired final concentration. 1 M NaOH was used to normalize pH in a volume proportional to collagen required at each tested concentration (pH 7.0, 10-20 μl 1 M NaOH), and the mixture was placed in 48 well culture plates and let polymerize at 37 °C. Final gel volumes were 200 μL.

Cell tracking and motility analysis. Cells were embedded in 3D collagen matrices in 48 well plates and left polymerize for 1 hour in a standard tissue culture incubator and then 200 μL of complete growth medium were added on top of the gels. The gels were transferred to a microscope stage top incubator and cells were imaged at low magnification (×10) every 2 minutes for 48 h. Coordinates of the cell location at each time frame were determined by tracking single cells using image recognition software (Metamorph/Metavue, Molecular Devices, Sunnyvale, CA). Tracking data was processed using custom written python scripts based on previously published scripts to calculate cell speed, invasion distances and Mean Squared Displacements (MSDs). For cell motility analysis before and after division the time lapse videos were scanned to identify dividing cells within the imaging period and the division point was identified as the frame at which a clear separation could be identified between daughter cells. The dividing cell was tracked up to the division point and one of the daughter cells (randomly chosen) was tracked from that point until the 48 h time point. For collective cell invasion distance the 48 h time lapse video was processed to obtain the maximum intensity projection (MIP), which highlights the tracks taken by the cells/groups of cells. Individual tracks distinguishable in the MIP were measured to obtain an equivalent invasion distance. All cell tracking data comes from 3 independent experiments performed on different days and with different cell passages.

Persistence random walk model implementation. To quantify the differences in the mean squared displacement (MSDs) the MSDs were fitted for each condition using the persistent random walk model (PRW model) as described previously in the art. Briefly, the MSDs were calculated as in Equation 1. The Equation 2 describing the PWR was fitted using python's Imfit library for each MSD. The persistent time (parameter P) was then extracted to calculate differences between groups as presented in FIG. 1B.

MSD(τ)= [symbol] (x(++π)−x(t))²+(y(t+τ)−y(t))² [symbol] Equation 1.

Where x and y are que coordinates of the position of a cell at each time point and tau is the time lag.

$$MSD ( τ ) = 2 {S}^{2} P ( τ - P ( 1 - {e}^{\frac{- τ}{P}} ) ) + 4 {σ}^{2} . \qquad Equation 2$$

Where, S is the cell speed and P is the persistence time and 8 is a function of the error in the position of the cell as described previously in the art.

Collagen stiffness modification and measurement using shear rheology. To modify the stiffness of collagen matrices without increasing density of material, 2.5 mg/mL gels at 20 °C for 30 minutes were kept until they were fully polymerized. After the initial polymerization the gels were placed on a humidified tissue culture incubator at 37 °C for at least 1 hour extra before adding cell growth media on top. To measure the effect of polymerization temperature on the gel stiffness the polymerization conditions were recreated for rheology testing (hybrid rheometer (DHR-2) from TA Instruments, New Castle, DE) using a cone and plate geometry with a sample volume of 0.6 mL. Shear storage modulus G′ was measured as reported before. Briefly, a strain sweep was performed from 0.1% to 100% strain at a frequency of 1 rad/s to determine the elastic region. Then a frequency sweep was performed at a strain within the linear region (0.8%) between 0.1-100 rad/s. Three independent replicates were performed for each condition tested.

Collagen structure modification using Poly-Ethylene-Glycol. To modify the structure if the collagen fibers within the gels without changing the final collagen concentration, Polyethylene glycol (PEG, MW-8000, Sigma, St. Louis, MO) was solubilized in phosphate-buffered solution (PBS), filter sterilized. Solubilized PEG was then mixed into the cells, reconstitution buffer solution described above to produce a final PEG concentration of 10 mg/mL in the collagen gel. The gels were allowed to polymerized in the same conditions as collagen only gels. Collagen structure modification was verified using confocal reflection microscopy.

RNA Isolation and purification. 3D collagen I gels were seeded in three independent experiments and harvested after 24 hours of culture for RNA extraction and directly homogenized in Trizol reagent (Thermofisher, Waltham, MA). Total RNA was isolated following manufacturer's instructions. Isolated RNA was further purified using High Pure RNA Isolation Kit (ROCHE, Branford, CT). RNA integrity was verified using RNA Analysis Screen Tape (Agilent Technologies, La Jolla, CA) before sequencing.

RNA sequencing and data analysis. Biological triplicates of total RNA were prepared for sequencing using the TruSeq Stranded mRNA Sample Prep Kit (Illumina, San Diego, CA) and sequenced on the Illumina MiSeq platform at a depth of >25 million reads per sample. The read aligner Bowtie2 was used to build an index of the reference human genome hg19 UCSC and transcriptome. Paired-end reads were aligned to this index using Bowtie241 and streamed to eXpress42 for transcript abundance quantification using command line “bowtie2 −a −p 10 −x/hg19 −1 reads_R1.fastq −2 reads_R2.fastq|express transcripts_hg19.fasta”. For downstream analysis TPM was used as a measure of gene expression. A gene was considered detected if it had mean TPM>5.

Gene ontology term overrepresentation analysis. To assess the overrepresented GO terms the cytoscape app BiNGO was used. Statistical test used was hypergeometric test, Benjamini-Hochberg false discovery rate (FDR) correction was used to account for multiple tests and the significance level was set at 0.05.

HIFIA Gene expression using qPCR. For qPCR experiments RNA was extracted as stated above and cDNA was synthesized using superscript iii first-strand synthesis system (Thermofisher, Waltham, MA). Relative mRNA levels were quantified using predesigned TaqMan gene expression assays (Thermofisher, Waltham, MA). Relative expression was calculated using the DCt method using GAPDH as reference gene. Assays used were: GAPDH (Hs02758991_g1), HIF1A (Hs00153153_m1).

CRISPR Mediated gene Knock-out: The lentiCRISPR v2 was a gift from Feng Zhang (Addgene plasmid #52961). Small guide RNAs targeting the genes of interest were cloned into the lentiCRISPR v2 following Zhang's lab instructions. The sg_RNA sequences using were taken from the GECKO human library A44. Used sequences were: ITGB1 sg_RNA1 (5′-TGCTGTGTGTTTGCTCAAAC-3′) (SEQ ID NO.: 1), ITGB1 sg_RNA2 (5′-ATCTCCAGCAAAGTGAAACC-3′) (SEQ ID NO.: 2), EGFP sgRNA (5′-GGGCGAGGAGCTGTTCACCG-3′) (SEQ ID NO.: 3). The lentiCRISPR v2 vectors with the cloned desired sgRNA were sequence verified and viral particles were generated by transfecting into lentiX293T cells (Clonetech, Mountain View, CA. Cat #632180) along with packaging expressing plasmid (psPAX2, Addgene #12260) and envelope expressing plasmid (pMD2.G, Addgene #12259). Viral particles were collected at 48 h after transfection and they were purified by filtering through a 0.45 μm filter. Target cells were transduced with the viral particles in the presence of polybrene (Allele Biotechnology, San Diego, CA). After overnight incubation media was changed and cells were left 24 h-48 h in normal growth media and then changed to puromycin selection media (2.5 μg/mL puromycin) for 7 days before experiments were performed.

Immunofluorescence and cell imaging. For cell imaging after 7 days of culture to visualize VM structures collagen gels were fixed using 2 washes of 4% PFA for 30 mins each at room temperature. F-actin was stained using Alexa Fluor® 488 Phalloidin (Cell signaling technology, Danver, MA) and the nuclei were counterstained with DAPI. For immunofluorescence staining the gels were incubated with the primary antibody for 48 to 72 hours. Anti-COL4A1 (1:200 dilution, NB120-6586, novus biologicals).

Confocal reflection imaging and quantification: Confocal reflection images were acquired using a Leica SP5 confocal microscope (Buffalo Grove, IL) equipped with a HCX APO L 20×1.0 water immersion objective. The sample was excited at 488 nm and reflected light was collected without an emission filter. For the estimation of pore size, modification of a previously reported digital imaging processing technique was used. Briefly, the images were normalized to account for uneven illumination effects. Then a threshold was applied to generate a binary mask where pores were identified as the darkest areas of the image. Pore diameter was measured using NIS elements software (Nikon Instruments Inc., Melville, NY) measure objects tool.

Western blotting: Cells were grown to >90% confluency in 100 mm dishes. After washing 2× with PBS cells were collected into 100 μL of lysis buffer with 1× Halt protease inhibitor cocktail (Pierce IP lysis Buffer, Thermofisher, Waltham, MA) by thoroughly scraping the dish surface. Cell lysate was incubate in ice with constant shaking for 30 min and then centrifuged at 15,000×g for 20 for protein purification. Samples were loaded at 50 ug total protein concentration for SDS-PAGE. Membranes were probed with antibodies against ITGB1 (#4706 from Cell signaling technology, Danver, MA. 1:10000 dilution) and aTubulin (TU-01 MA1-19162, Thermofisher, Waltham, MA. 1:30000 dilution).

Experimental data analysis and statistics: All cell motility data was analyzed for statistical significance using the scipy python package. Experimental data in FIGS. 3 and 4 was analyzed using prism graphpad (San Diego, CA). Significance (p) was indicated within the figures using the following scale: * p<0.05 ** p<0.01; *** p<0.001.

TCGA data reprocessing and survival analysis: The TCGA raw data were downloaded from CGHub directly using gtdownload. Corresponding clinical metadata were obtained from the TCGA data portal (tcga423data.nci.nih.gov/docs/publications/tcga/). RNAseq fastq files were realigned and quantified using sailfish v.0.7.6 with default parameters. Only primary tumors were considered in the analysis. In the analysis of breast invasive carcinoma, only the patients with reported histological staining for the three markers (Her2, ER, PR) could be associated with a molecular subtype. Patients for which any of the histological markers were not evaluated or were detected at an equivocal level were assigned to an “unknown” subtype. TCGA data for Stage I, II, III and IV breast cancer patients was analyzed by Principal Component Analysis (PCA) with respect to the 70 VM genes to construct gene expression meta-markers as previously described47. PCA-based score quantiles were mapped to VM high and VM low categories based on mean VM gene expression levels. Because the VM signature comprised only genes that were upregulated in the presence of the VM phenotype, the overall mean expression of VM genes was used to map PCA score to VM signature activity level.

TCGA pan cancer analysis. Tumor types for which at least 100 patients had both expression and clinical metadata were analyzed to determine correlation between a VM gene expression and 5-year survival. Only primary tumors were considered. Kaplan-Meier analysis was performed comparing the 30% of individuals with the lowest VM expression score to the 30% with the highest score using the Lifelines python library (lifelines.readthedocs.io/en/latest/). The log rank test was used to determine significance of survival differences between groups.

### Example 3

### 3D Collagen Architecture Induces a Conserved Migratory and Transcriptional Response Linked to Vasculogenic Mimicry

The topographical organization of collagen within the tumor microenvironment has been implicated in modulating cancer cell migration and independently predicts progression to metastasis. This example shows that collagen matrices with small pores and short fibers, but not Matrigel, trigger a conserved transcriptional response and subsequent motility switch in cancer cells resulting in the formation of multicellular network structures. The response is not mediated by hypoxia, matrix stiffness, or bulk matrix density, but rather by matrix architecture-induced β1 integrin upregulation. The transcriptional module associated with network formation is enriched for migration and vasculogenesis-associated genes that predict survival in patient data across nine distinct tumor types. Evidence of this gene module at the protein level is found in patient tumor slices displaying a vasculogenic mimicry (VM) phenotype. These findings link a collagen-induced migration program to VM and support the conclusion that this process is broadly relevant to metastatic progression in solid human cancers.

An initial step in cancer metastasis is the migration of tumor cells through the extracellular matrix (ECM) and into the lymphatic or vascular systems. Several features of the tumor ECM have been associated with progression to metastasis. In particular, regions of dense collagen are co-localized with aggressive tumor cell phenotypes in numerous solid tumors, including breast, ovarian, pancreatic, and brain cancers. However, sparse and aligned collagen fibers at the edges of tumors have also been reported to correlate with aggressive disease. It remains unclear whether and how collagen architectures play a role in driving metastatic migration programs or if they simply correlate with progression of the tumor.

Intravital microscopy studies have shown that distinct collagen architectures are associated with specific cell motility behaviors. Cancer cells migrating through densely packed collagen within the tumor use invadopodia and matrix metalloproteinase (MMP) activity to move, whereas cells in regions with less dense collagen and long, aligned fibers migrate rapidly using larger pseudopodial protrusions or MMP-independent amoeboid blebbing. Cell migration speed, invasion distance, and cellular protrusion dynamics are modulated by collagen fiber alignment, but that this relationship breaks down at high collagen densities (>2.5 mg mL⁻¹). Without being bound by theory, these findings suggest that distinct motility regimes exist in low and high density collagen, which may have implications for metastatic progression.

The relationships between collagen density, collagen architecture, cell migration behavior, gene expression and metastatic potential were explored by developing a 3D in vitro model system designed to probe the physical basis of cancer cell migration responses to collagen matrix organization. Using this system, it was found that confining collagen matrix architectures with short fibers and small pores induced a conserved migration behavior in cancer cells leading to network formation and the upregulation of a conserved transcriptional module, both of which are mediated by integrin μ1 upregulation. Without being bound by theory, this evidence shows that this in vitro behavior is consistent with phenotypic and molecular features of clinical VM. Moreover, without being bound by theory, the evidence showed that the associated transcriptional response is conserved among cancer types in vitro and is predictive of patient survival in multiple clinical datasets for various tumor types. This integrative study supports the conclusion that a collagen induced migration phenotype and gene expression program are linked to a metastatic clinical tumor cell phenotype.

### High Density Collagen Promotes Fast and Persistent Migration

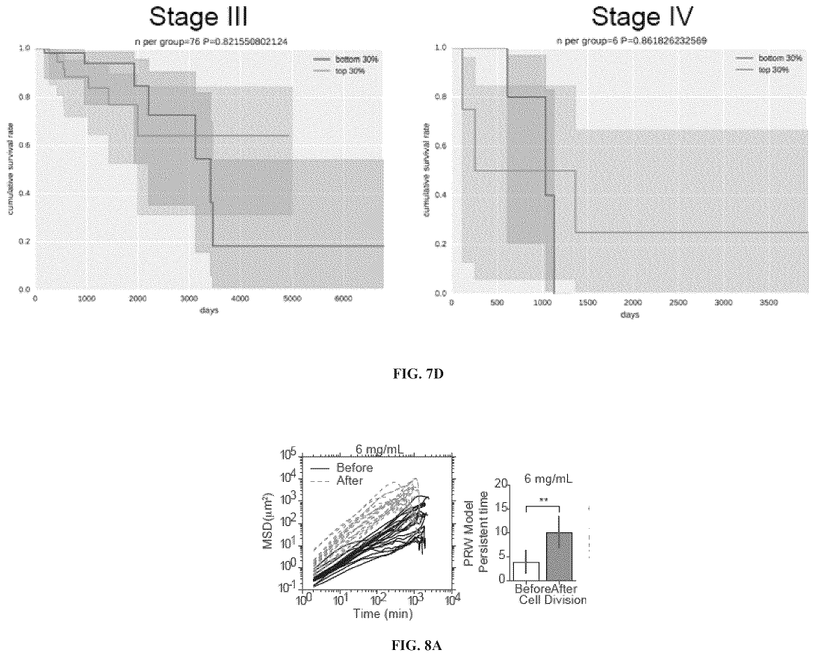

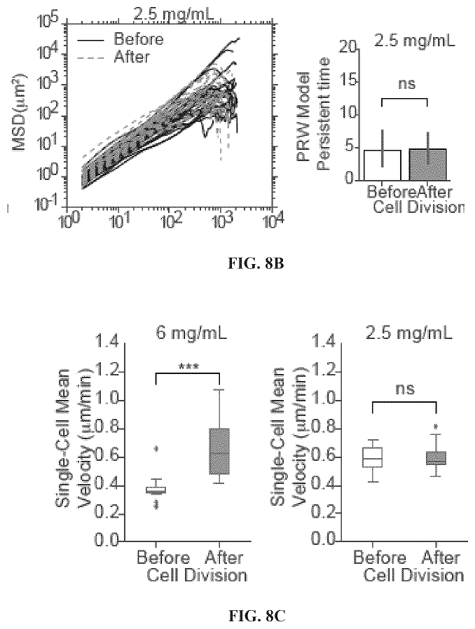

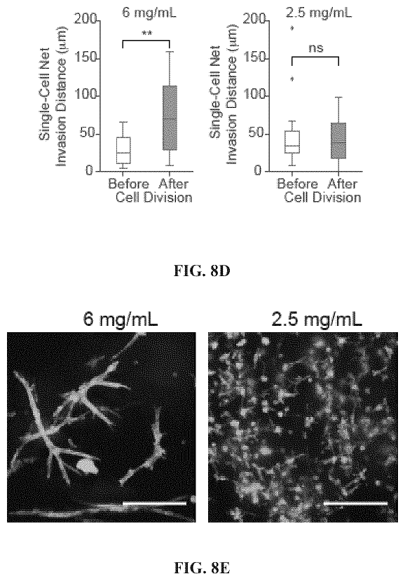

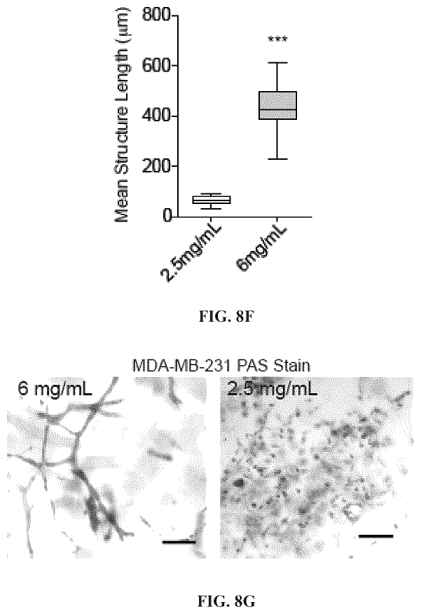

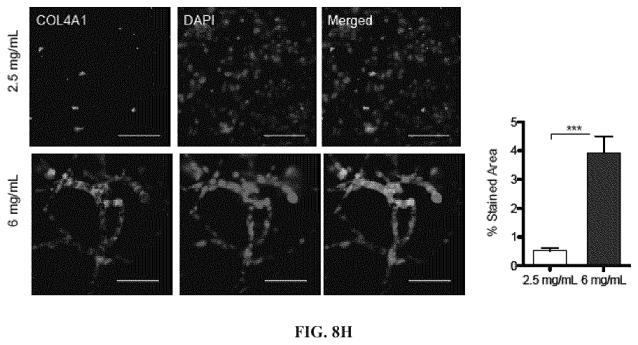

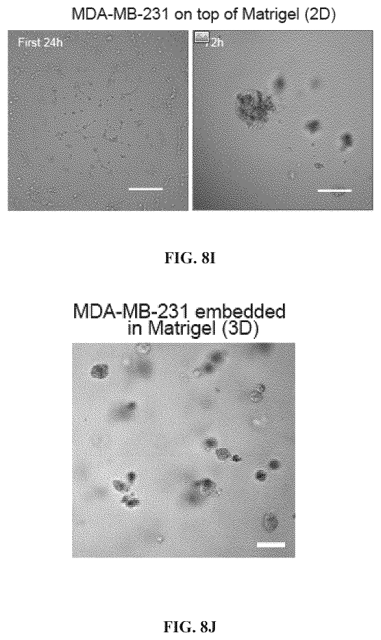

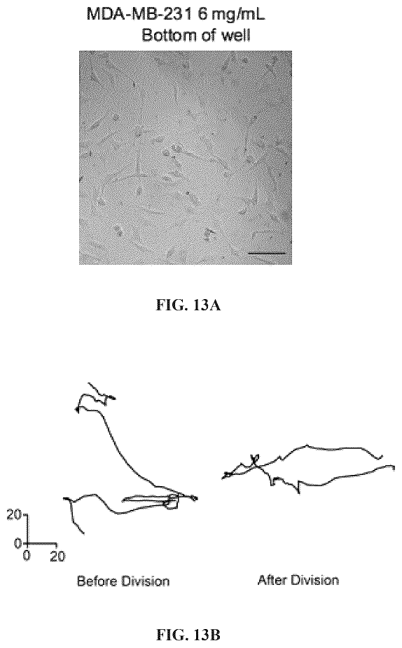

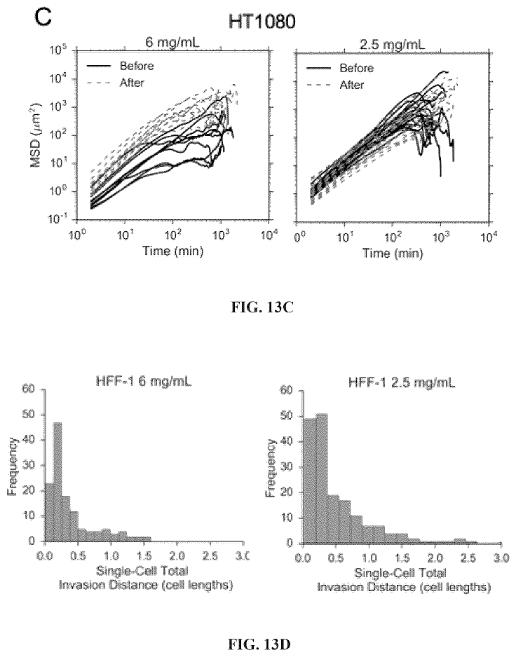

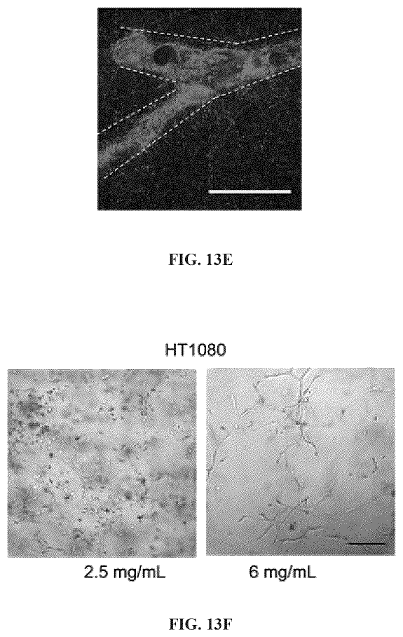

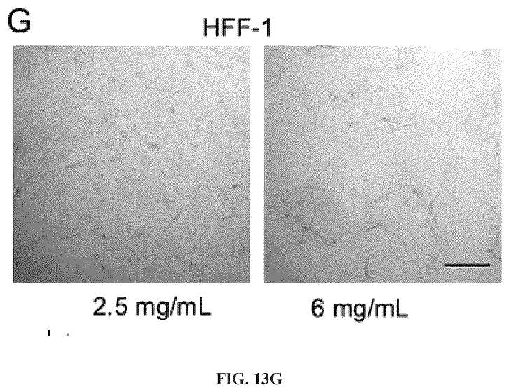

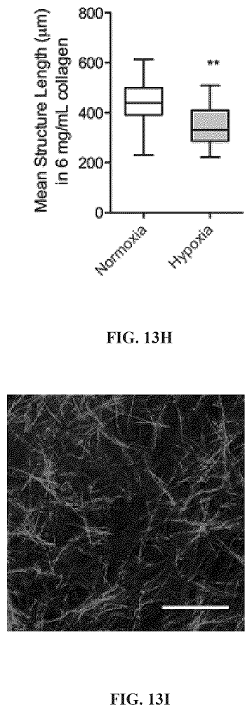

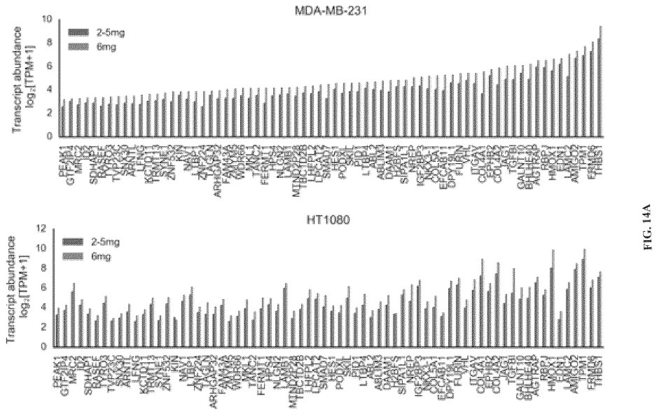

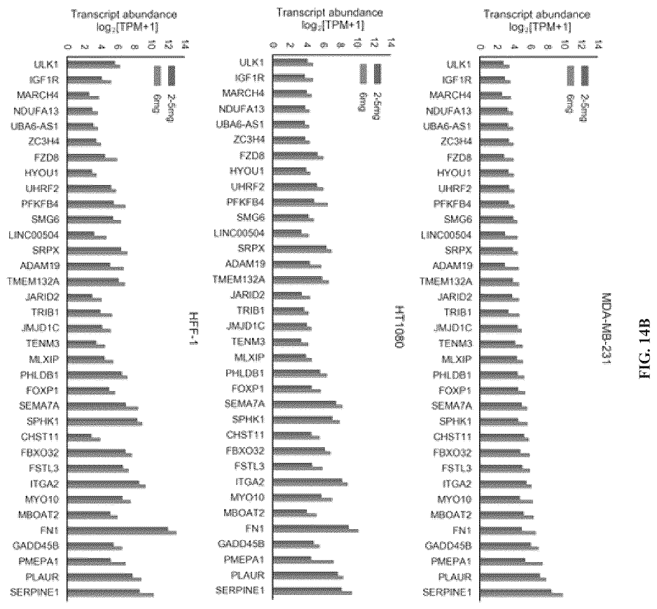

To first investigate the role of 3D collagen density in modulating the migration phenotype of breast cancer cells, MDA-MB-231 cells were embedded in collagen I matrices at densities mimicking normal breast tissue, 2.5 mg/mL collagen, and cancerous breast tissue, 6 mg mL⁻¹ collagen. Cells migrating in dense collagen initially appeared to be trapped and were unable to invade. However, after one division cycle, most cells switched to a highly invasive motility behavior, significantly increasing their persistence, velocity, and total invasion distance (FIGS. 8A-D, left panels). This behavior was not observed in cells embedded in the low density matrix, where cell migration was the same before and after division (FIGS. 8A-D, right panels). Interestingly, cells that were in contact with the coverslip and not fully embedded in the high density condition did not undergo the same migration transition upon division (FIGS. 13A-B). The motility responses in 2.5 and 6 mg mL⁻¹ collagen matrices were not unique to MDA-MB-231 breast cancer cells. Similar migration patterns were observed for HT-1080 fibrosarcoma cells embedded in the same collagen matrix conditions (FIG. 13C), suggesting that these responses may be conserved among distinct cancer types. To further examine whether the observed migration behavior was cell type dependent, the response of normal mesenchymal human foreskin fibroblasts (HFF-1) to low and high density collagen conditions was tested. Over an observation period of 48 h, HFF cells migrated consistently with very low persistence. Cells invaded less than three cell lengths in low density collagen. In high density, HFFs elongated to reach cell lengths up to 300 μm but did not invade significantly (FIG. 13D).

### Density-Induced Migration Results in Cell Network Structures

It was unexpected that both MDA-MB-231 and HT-1080 cancer cells migrated faster and further in high density collagen conditions. Intuitively, cell migration would be expected to slow in dense conditions where more matrix must be remodeled to enable cell movement. Moreover, this behavior was common to both cancer cell types but not displayed by normal fibroblasts, which represent residents of the stroma and also undergo mesenchymal migration in collagen. This motivated us to investigate the long-term implications of the rapid migration phenotype induced in cancer cells under high density conditions. After one week of culture in high density collagen, breast cancer cells undergoing rapid and persistent migration formed interconnected network structures that resembled the early stages of endothelial tubulogenesis (FIG. 8E, left). The average length of cell networks after one week was 437 m (FIG. 8F). Interestingly, these network structures do not appear to be caused by cells aligning along collagen fibers (FIG. 13E). In contrast, cells cultured in low density collagen for one week migrated slowly with low persistence, and remained as single cells (FIG. 8E, right). HT-1080 cells also formed network structures in high density collagen and remained as single cells in low density collagen (FIG. 13F). HFFs remained as single cells in both high and low density conditions (FIG. G). The transition of cancer cells from single cell migration to network formation suggested a potential transdifferentiation event, and the cell networks were reminiscent of a cancer phenotype known as vasculogenic mimicry (VM). VM is thought to arise from tumor cells that acquire the ability to form networks in the tumor ECM lined with glycogen rich molecules and basement membrane proteins that can be perfused with blood. However, the tumor cells lining these networks do not express endothelial surface markers such as CD31. Periodic acid Schiff (PAS) staining of the networks formed in the high density collagen condition confirmed the presence of glycogen rich molecules (FIG. 8G) and immunofluorescence confirmed the presence of basement membrane protein COL4A1 (FIG. 8H), as in VM.

Previous pioneering studies have shown that several aggressive melanoma cell lines which produce VM in vivo also intrinsically form VM network structures when cultured on top of Matrigel or collagen I in a 2D in vitro context. Recently, other aggressive tumor cell types have been shown to intrinsically form VM-like network structures on top of Matrigel or in 2.5D culture in Matrigel. Here, it is important to note that variations exist in the consistency of commercial ECM products as well as the terminology used to describe 3D culture. In this example, 3D culture is defined strictly as a condition where cells are fully embedded, in contact with ECM on all sides, and located a sufficient distance away from the coverslip bottom and sides of the culture dish to avoid their influence. 2.5D culture is defined as a pseudo 3D culture where cells are embedded in the ECM but in contact with coverslip. To understand whether the network phenotype induced by a 3D collagen I environment was distinct from that induced by a 2D Matrigel environment, experiments were performed to determine whether the cells formed network structures on top of Matrigel. Few cells aligned within the first 24 hrs of culture, and nearly all cells aggregated after 72h (FIG. 8I). Next MDA-MB-231 cells were embedded inside of Matrigel, in 3D culture. In this context, cells did not form network structures but instead formed rough-edged, disorganized spheroids (FIG. 8J). Thus, high density collagen uniquely induced the network forming phenotype in a more physiologically relevant 3D context.

### a Conserved Transcriptional Response Precedes Migration

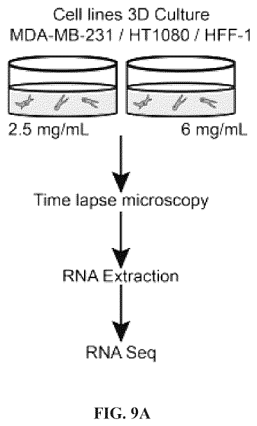

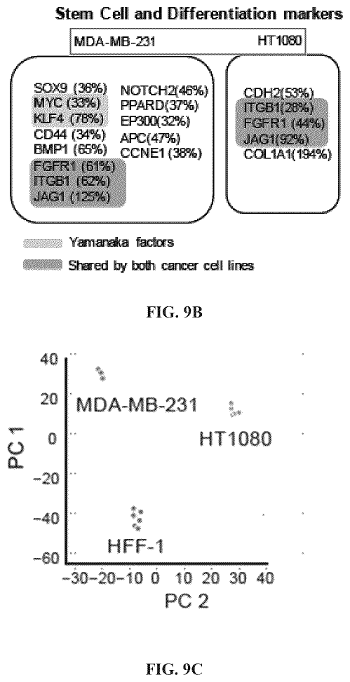

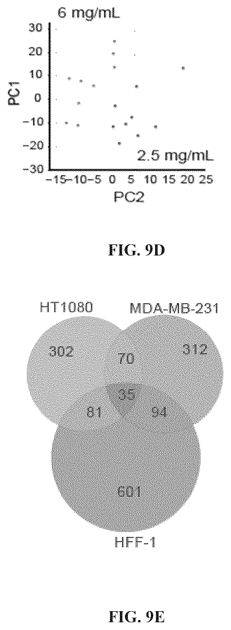

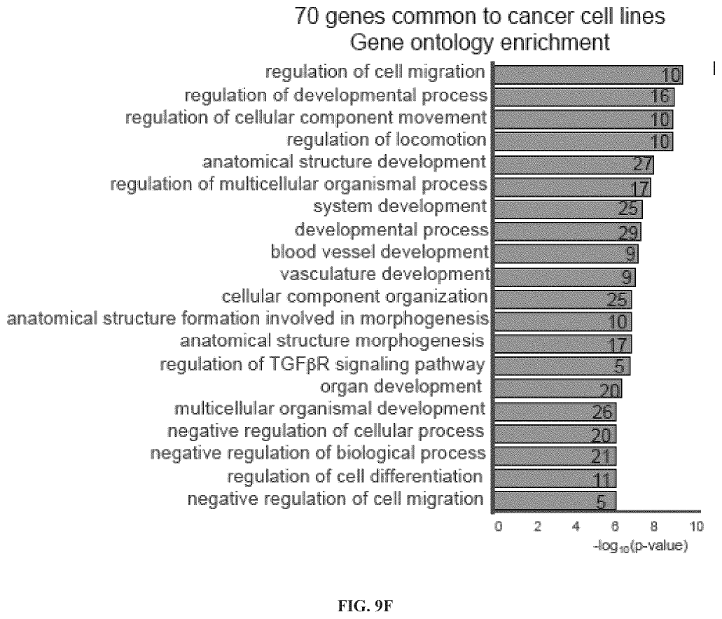

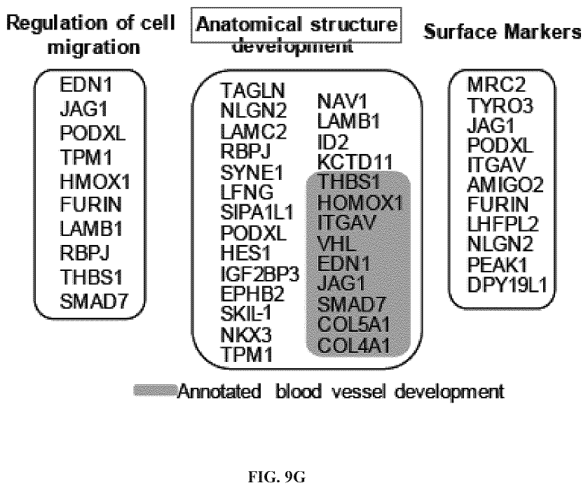

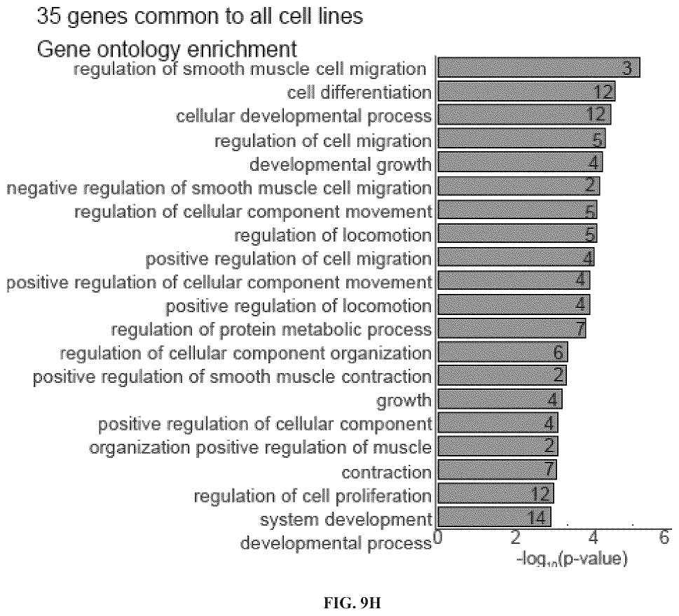

Without being bound by theory, it is believed that the persistent migration phenotype of cancer cells leading to network formation in high density collagen conditions (collagen induced network phenotype, CINP) is the result of a transdifferentiation event wherein a unique cell motility gene module was upregulated. To test this, RNA sequencing was performed of MDA-MB-231, HT-1080, and HFF cells cultured in low and high density collagen matrices after 24 hours (FIG. 9A), the time point just before most cancer cells in the high density collagen matrix underwent at least one cycle of cell division and began to invade with increased persistence. Since the majority of cancer cells cultured under high density conditions participated in network formation, it was expected that their bulk transcriptional profile would be dominated by this phenotype. Analysis was performed to determine if common stem cell and differentiation markers were upregulated in association with the network forming phenotype. Indeed, several known stem cell markers were upregulated (FIG. 9B), and three were common to both cancer cell types: JAG1, ITGB1, and FGFR1. Without being bound by theory, this data supports the conclusion that both cancer cell types harbored stem-like qualities, which could facilitate significant transcriptional reprogramming.

Analyzing more broadly, it was asked which genes were differentially regulated (TPM Fold change >=1.5) in high density collagen compared to low density collagen in each cell type and whether these genes represented unique or conserved transcriptional response modules. As expected, cell type accounted for the most variance in gene expression (FIG. 9C). However, after a z-score transformation of the gene expression of each cell type, the collagen matrix condition accounted for the bulk of the remaining variance in gene expression (FIG. 9D). This suggested the presence of gene expression programs linked to collagen matrix conditions.

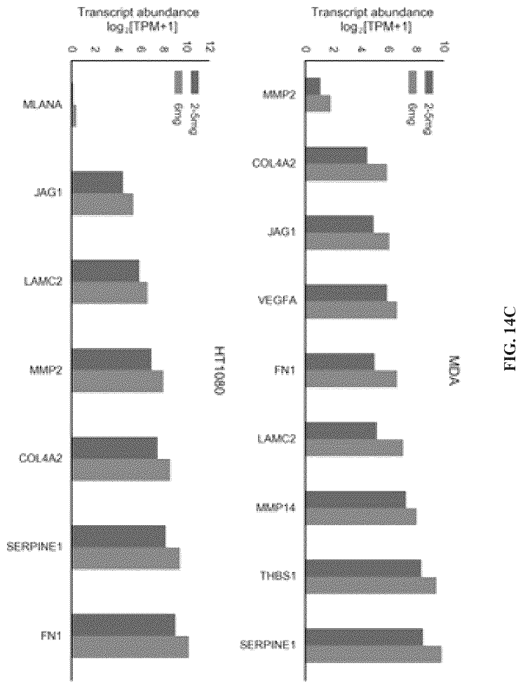

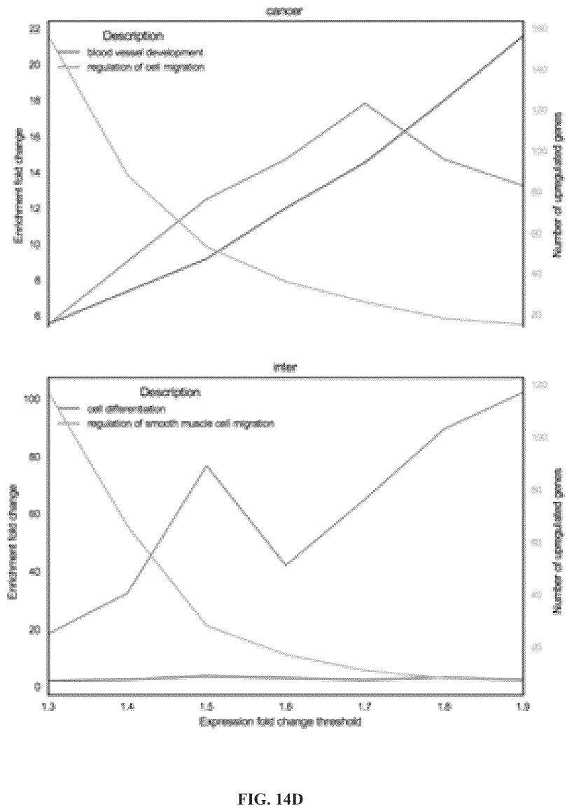

Using a Venn Diagram approach to identify conserved expression modules, a set of 70 genes was discovered that were upregulated by both cancer cell types but not normal cells in response to high density collagen (FIG. 9E, FIG. 14A). Gene ontology (GO) enrichment analysis revealed that these 70 common-to-cancer genes were significantly enriched for annotations in blood vessel development and regulation of migration (FIGS. 9 F and 9G). Importantly, changes in the threshold for differential expression did not significantly alter the primary gene ontology categories identified (FIG. 14D and Table 2). Key genes involved in Notch signaling, i.e. RBPJ and LFNG, were among the 70. Importantly, LAMC2, JAG1, and THBS1 genes identified in this common-to-cancer gene set have been previously associated with a VM phenotype intrinsically displayed by metastatic melanoma, which was assessed by targeted microarray analysis for angiogenesis, ECM, and cell adhesion genes. Upregulated surface markers were not endothelial in nature, and did not represent any specific tissue or cell type (FIG. 9G).

TABLE 2
Sensitivity analysis of GO enrichment

| | | | # | | |
|---|---|---|---|---|---|
| | Gene | | genes | expec- | Fold |
| fcThreshold | List | Description | in set | tation | enrich |
| 1.3 | 70 Genes | blood vessel | 16 | 2.890 | 5.536 |
| | | development | | | |
| | | regulation of cell | 13 | 2.356 | 5.518 |
| | | migration | | | |
| | 35 Genes | cell differentiation | 28 | 13.647 | 2.052 |
| | | regulation of smooth | 3 | 0.164 | 18.333 |
| | | muscle cell migration | | | |
| 1.4 | 70 Genes | blood vessel | 12 | 1.630 | 7.361 |
| | | development | | | |
| | | regulation of cell | 12 | 1.329 | 9.030 |
| | | migration | | | |
| | 35 Genes | cell differentiation | 19 | 7.696 | 2.469 |
| | | regulation of smooth | 3 | 0.092 | 32.511 |
| | | muscle cell migration | | | |
| 1.5 | 70 Genes | blood vessel | 9 | 0.982 | 9.167 |
| | | development | | | |
| | | regulation of cell | 10 | 0.800 | 12.496 |
| | | migration | | | |
| | 35 Genes | cell differentiation | 12 | 3.265 | 3.676 |
| | | regulation of smooth | 3 | 0.039 | 76.639 |
| | | muscle cell migration | | | |
| 1.6 | 70 Genes | blood vessel | 8 | 0.667 | 11.997 |
| | | development | | | |
| | | regulation of cell | 8 | 0.544 | 14.718 |
| | | migration | | | |
| | 35 Genes | cell differentiation | 6 | 1.982 | 3.027 |
| | | regulation of smooth | 1 | 0.024 | 42.076 |
| | | muscle cell migration | | | |
| 1.7 | 70 Genes | blood vessel | 7 | 0.482 | 14.534 |
| | | development | | | |
| | | regulation of cell | 7 | 0.393 | 17.832 |
| | | migration | | | |
| | 35 Genes | cell differentiation | 3 | 1.283 | 2.339 |
| | | regulation of smooth | 1 | 0.015 | 65.027 |
| | | muscle cell migration | | | |
| 1.8 | 70 Genes | blood vessel | 6 | 0.333 | 17.995 |
| | | development | | | |
| | | regulation of cell | 4 | 0.272 | 14.718 |
| | | migration | | | |
| | 35 Genes | cell differentiation | 3 | 0.933 | 3.216 |
| | | regulation of smooth | 1 | 0.011 | 89.413 |
| | | muscle cell migration | | | |
| 1.9 | 70 Genes | blood vessel | 6 | 0.278 | 21.594 |
| | | development | | | |
| | | regulation of cell | 3 | 0.226 | 13.246 |
| | | migration | | | |
| | 35 Genes | cell differentiation | 2 | 0.816 | 2.450 |
| | | regulation of smooth | 1 | 0.010 | 102.186 |

muscle cell migration

Further exploration of this dataset with respect to individual cancer cell types revealed that, beyond the conserved transcriptional response, high density collagen also triggered the expression of genes related to vasculogenesis in a cell type dependent manner. For example, breast cancer cell networks upregulated VEGFA fold change=1.65 and MMP14 fold change=1.72, but fibrosarcoma cell networks did not. Some of these genes have been previously associated with the VM network phenotype of melanoma cells (FIG. 14C).

Next the thirty-five genes that were upregulated in response to high density collagen by all three cell types was assessed (FIG. 9E). These genes were enriched primarily for annotations in regulation of cell differentiation (FIG. 9H). However, it is important to take into account the inherent flaws associated with GO enrichment analysis. For example, some categories showing enrichment in the 35 genes common to all cell lines contain very few genes and may not represent real enrichment. However, this limitation is not observed in the top enriched categories in the 70 genes common to cancer cells, where most category contain at least 10 genes (FIG. 9F). The genes associated with each enrichment category are given in Tables 3 and 4.

TABLE 3
Gene ontology enrichment analysis for the genes in the 70 gene list

| | # genes | |
|---|---|---|
| GO Term | in set | genes in set |
| regulation of cell | 10 | EDN1\|JAG1\|PODXL\|TPM1\|HMOX1\| |
| migration | | FURIN\|LAMB1\|RBPJ\|THBS1\|SMAD7 |
| regulation of | 16 | EDN1\|JAG1\|LTBP4\|HPS4\|THBS1\| |
| developmental | | SMAD7\|SIPA1L1\|COL4A2\|ID2\| |
| process | | HMOX1\|ITGAV\|HES1\|VHL\|EPHB2\| |
| | | SKIL\|NKX3-1 |
| regulation of | 10 | EDN1\|JAG1\|PODXL\|TPM1\|HMOX1\| |
| cellular component | | FURIN\|LAMB1\|RBPJ\|THBS1\|SMAD7 |
| movement | | |
| regulation of | 10 | EDN1\|JAG1\|PODXL\|TPM1\|HMOX1\| |
| locomotion | | FURIN\|LAMB1\|RBPJ\|THBS1\|SMAD7 |
| anatomical structure | 27 | TAGLN\|NLGN2\|LAMC2\|RBPJ\| |
| development | | THBS1\|SYNE1\|LFNG\| |
| | | SIPA1L1\|PODXL\|HMOX1\| |
| | | ITGAV\|HES1\|IGF2BP3\|VHL\| |
| | | EPHB2\|SKIL\|NKX3- |
| | | 1\|EDN1\|JAG1\|TPM1\|NAV1\| |
| | | LAMB1\|SMAD7\|COL5A1\| |
| | | COL4A1\|ID2\|KCTD11 |
| regulation of | 17 | EDN1\|NLGN2\|JAG1\|TPM1\|FURIN\| |
| multicellular | | THBS1\|SMAD7\|SIPA1L1\| |
| organismal process | | COL4A2\|ID2\|BHLHE40\|HMOX1\| |
| | | HES1\|IGF2BP3\|EPHB2\| |
| | | SKIL\|NKX3-1 |
| system development | 25 | TAGLN\|NLGN2\|LAMC2\|RBPJ\| |
| | | THBS1\|LFNG\|SIPA1L1\|PODXL\| |
| | | HMOX1\|ITGAV\|HES1\|VHL\|EPHB2\| |
| | | SKIL\|NKX3-1\|EDN1\|JAG1\|TPM1\| |
| | | NAV1\|LAMB1\|SMAD7\|COL5A1\|CO |
| | | L4A1\|ID2\|KCTD11 |
| developmental | 29 | TAGLN\|NLGN2\|LTBP4\|LAMC2\| |
| process | | FURIN\|RBPJ\|THBS1\|SYNE1\| |
| | | LFNG\|SIPA1L1\|PODXL\|HMOX1\| |
| | | ITGAV\|HES1\|IGF2BP3\| |
| | | VHL\|EPHB2\|SKIL\|NKX3-1\| |
| | | EDN1\|JAG1\|TPM1\|NAV1\|LAMB1\| |
| | | SMAD7\|COL5A1\|COL4A1\|ID2\|KCTD11 |
| blood vessel | 9 | EDN1\|JAG1\|COL5A1\|COL4A1\| |
| development | | HMOX1\|ITGAV\|VHL\| |
| | | THBS1\|SMAD7 |
| vasculature | 9 | EDN1\|JAG1\|COL5A1\|COL4A1\|HMOX1\| |
| development | | ITGAV\|VHL\|THBS1\|SMAD7 |
| cellular component | 25 | NLGN2\|LAMC2\|RBPJ\|THBS1\| |
| organization | | SYNE1\|MRC2\|SIPA1L1\|ABLIM3\| |
| | | HMOX1\|ITGAV\|HES1\|VHL\|EPHB2\| |
| | | SKIL\|TPM1\|HPS4\|NAV1\|LAMB1\| |
| | | H2BFS\|SMAD7\|DAAM1\|COL4A2\| |
| | | COL5A1\|LPCAT2\|TGFBI |
| anatomical structure | 10 | EDN1\|JAG1\|COL4A1\|PODXL\|TPM1\| |
| formation involved | | HMOX1\|VHL\|THBS1\|SKIL\|NKX3-1 |
| in morphogenesis | | |
| anatomical structure | 17 | EDN1\|JAG1\|TPM1\|LAMB1\|THBS1\| |
| morphogenesis | | SMAD7\|LFNG\|COL5A1\|COL4A1\| |
| | | PODXL\|HMOX1\|HES1\| |
| | | IGF2BP3\|VHL\|EPHB2\|SKIL\|NKX3-1 |
| regulation of | 5 | LTBP4\|FURIN\|THBS1\|SKIL\|SMAD7 |
| transforming | | |
| growth factor beta | | |
| receptor signaling | | |
| pathway | | |
| organ development | 20 | EDN1\|TAGLN\|JAG1\|TPM1\|LAMC2\| |
| | | LAMB1\|THBS1\|SMAD7\|LFNG\| |
| | | COL5A1\|COL4A1\|PODXL\|ID2\| |
| | | HMOX1\|ITGAV\|HES1\| |
| | | VHL\|EPHB2\|SKIL\|NKX3-1 |
| multicellular | 26 | TAGLN\|NLGN2\|LTBP4\|LAMC2\| |
| organismal | | RBPJ\|THBS1\|LFNG\| |
| development | | SIPA1L1\|PODXL\|HMOX1\|ITGAV\| |
| | | HES1\|VHL\|EPHB2\|SKIL\|NKX3-1\| |
| | | EDN1\|JAG1\|TPM1\|NAV1\|LAMB1\| |
| | | SMAD7\|COL5A1\|COL4A1\|ID2\|KCTD11 |
| negative regulation | 20 | EDN1\|JAG1\|TPM1\|AMIGO2\| |
| of cellular process | | FURIN\|RBPJ\|THBS1\|SMAD7\| |
| | | PODXL\|ID2\|BHLHE40\|HMOX1\| |
| | | ITGAV\|HES1\|IGF2BP3\| |
| | | VHL\|TGFBI\|EPHB2\|SKIL\|NKX3-1 |
| negative regulation | 21 | EDN1\|JAG1\|TPM1\|AMIGO2\| |
| of biological process | | FURIN\|RBPJ\|THBS1\| |
| | | SMAD7\|COL4A2\|PODXL\|ID2\| |
| | | BHLHE40\|HMOX1\|ITGAV\|HES1\| |
| | | IGF2BP3\|VHL\|TGFBI\|EPHB2\| |
| | | SKIL\|NKX3-1 |
| regulation of cell | 11 | EDN1\|SIPA1L1\|JAG1\|ID2\|LTBP4\| |
| differentiation | | ITGAV\|HES1\|VHL\| |
| | | EPHB2\|SKIL\|SMAD7 |

TABLE 4
Gene ontology enrichment analysis for the genes in the 35 gene list

| | #genes | |
|---|---|---|
| GO Term | in set | genes in set |
| regulation of smooth | 3 | ITGA2\|SERPINE1\|TRIB1 |
| muscle cell | | |
| migration | | |
| cell differentiation | 12 | SEMA7A\|UHRF2\|CHST11\| |
| | | GADD45B\|ITGA2\| |
| | | SPHK1\|FN1\|FZD8\|ULK1\| |
| | | JARID2\|FSTL3\|IGF1R |
| cellular | 12 | SEMA7A\|UHRF2\|CHST11\| |
| developmental | | GADD45B\|ITGA2\|SPHK1\| |
| process | | FN1\|FZD8\|ULK1\|JARID2\|FSTL3\|IGF1R |
| regulation of cell | 5 | ITGA2\|SPHK1\|SERPINE1\| |
| migration | | TRIB1\|IGF1R |
| developmental | 4 | CHST11\|SERPINE1\|PLAUR\|ULK1 |
| growth | | |
| negative regulation | 2 | SERPINE1\|TRIB1 |
| of smooth muscle | | |
| cell migration | | |
| regulation of | 5 | ITGA2\|SPHK1\|SERPINE1\| |
| cellular component | | TRIB1\|IGF1R |
| movement | | |
| regulation of | 5 | ITGA2\|SPHK1\|SERPINE1\| |
| locomotion | | TRIB1\|IGF1R |
| positive regulation | 4 | ITGA2\|SPHK1\|SERPINE1\|IGF1R |
| of cell migration | | |
| positive regulation | 4 | ITGA2\|SPHK1\|SERPINE1\|IGF1R |
| of cellular | | |
| component | | |
| movement | | |
| positive regulation | 4 | ITGA2\|SPHK1\|SERPINE1\|IGF1R |
| of locomotion | | |
| regulation of protein | 7 | NDUFA13\|ITGA2\|SPHK1\| |
| metabolic process | | SERPINE1\|PLAUR\|JARID2\|TRIB1 |
| regulation of | 6 | ITGA2\|SPHK1\|SERPINE1\|FN1\| |
| cellular component | | ULK1\|JARID2 |
| organization | | |
| positive regulation | 2 | ITGA2\|SPHK1 |
| of smooth muscle | | |
| contraction | | |
| growth | 4 | CHST11\|SERPINE1\|PLAUR\|ULK1 |
| positive regulation | 4 | ITGA2\|SPHK1\|SERPINE1\|JARID2 |
| of cellular | | |
| component | | |
| organization | | |
| positive regulation | 2 | ITGA2\|SPHK1 |
| of muscle | | |
| contraction | | |
| regulation of cell | 7 | CHST11\|ITGA2\|SPHK1\|SERPINE1\| |
| proliferation | | JARID2\|TRIB1\|IGFIR |

Cancer types for which there is data available in TCGA but no significant differences between high and low CINP groups were detected. Table 5 shows number of patients available, number of deaths reported, hazard ratio and cox model p value.

TABLE 5
TCGA analysis not significant effect cox p > 0.05

| Cancer type | Patient count | Death Observed | HR | Cox p |
|---|---|---|---|---|
| BRCA | 1131 | 104 | 1.1116 | 0.3268 |
| UCEC | 555 | 45 | 1.2440 | 0.1955 |
| HNSC | 518 | 167 | 1.1604 | 0.0748 |
| PRAD | 505 | 8 | 1.3992 | 0.4090 |
| THCA | 504 | 14 | 1.0754 | 0.8104 |
| COAD | 499 | 59 | 0.8250 | 0.1646 |
| LUSC | 489 | 154 | 1.0746 | 0.3913 |
| LIHC | 369 | 89 | 1.1355 | 0.2679 |
| OV | 337 | 185 | 1.0028 | 0.9721 |
| KIRP | 287 | 32 | 1.2456 | 0.2371 |
| STAD | 279 | 77 | 1.2858 | 0.0571 |
| SARC | 257 | 75 | 0.9090 | 0.4110 |
| PCPG | 179 | 6 | 0.8633 | 0.7016 |
| READ | 165 | 9 | 0.5978 | 0.3112 |
| GBM | 156 | 53 | 1.1312 | 0.2311 |
| TGCT | 133 | 3 | 0.9231 | 0.9001 |
| THYM | 120 | 6 | 1.0127 | 0.9496 |
| ESCA | 119 | 57 | 0.7878 | 0.6258 |
| SKCM | 93 | 10 | 1.6624 | 0.2016 |
| UVM | 80 | 13 | 1.4671 | 0.1305 |
| UCS | 57 | 25 | 0.8913 | 0.5450 |
| DLBC | 47 | 5 | 0.9887 | 0.9806 |
| CHOL | 36 | 16 | 1.0232 | 0.9343 |

Interestingly, SERPINE1, a secreted protease inhibitor involved in coagulation and inflammation regulation, was identified in the common-to-all gene module (FIG. 14B). Several Serpine protein family members have previously been implicated as drivers of metastasis correlating with VM and with brain metastases of lung and breast cancers.

### Integrin β1 Upregulation is Required for CINP

Identifying the matrix feature triggering transdifferentiation. The physical parameters of stiffness, pore size, and fiber organization differ between the low density 2.5 mg mL⁻¹ and high density 6 mg mL⁻¹ collagen matrices. Chemical cues may also change. For example adhesive ligand density and binding site presentation to integrins and other matrix receptors may differ as well as accumulation or release of autocrine and paracrine signals sequestered by the ECM. Each of these features could potentially impact cancer cell motility behavior and gene expression.

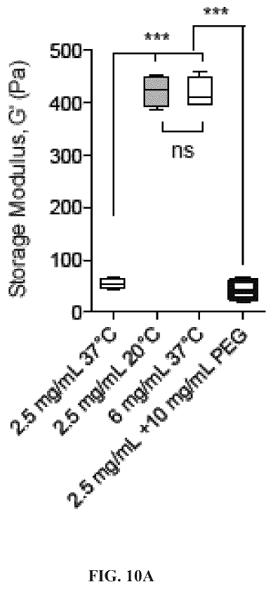

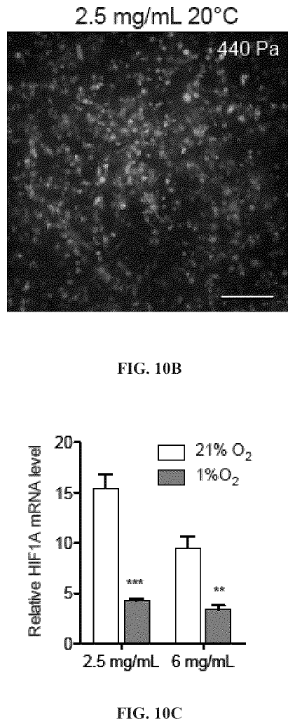

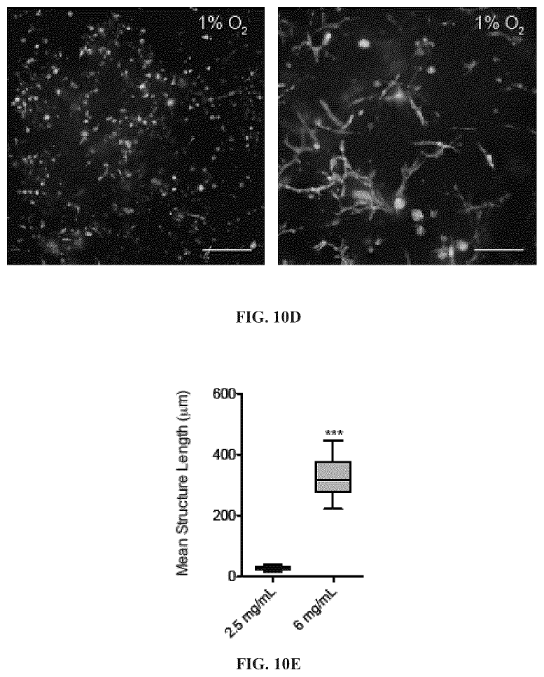

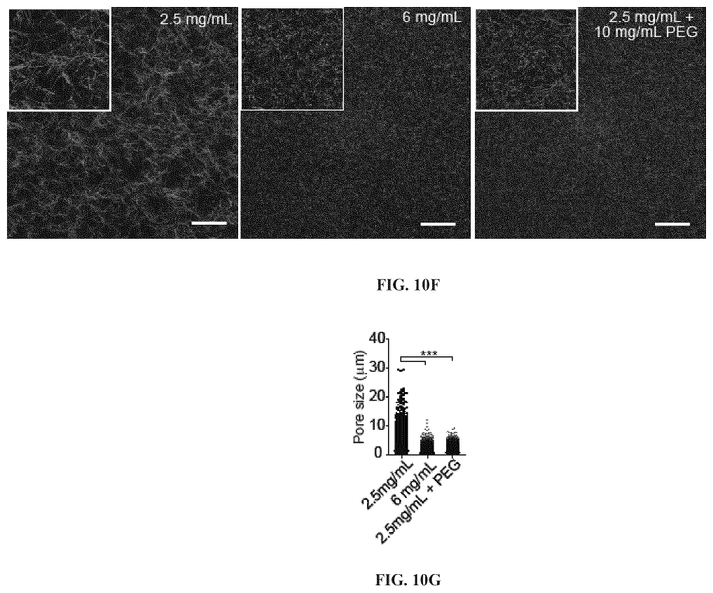

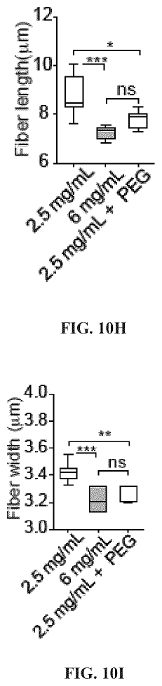

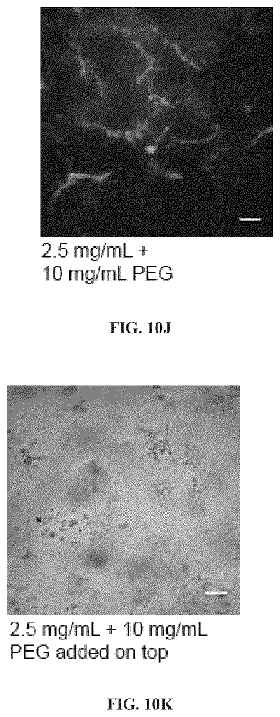

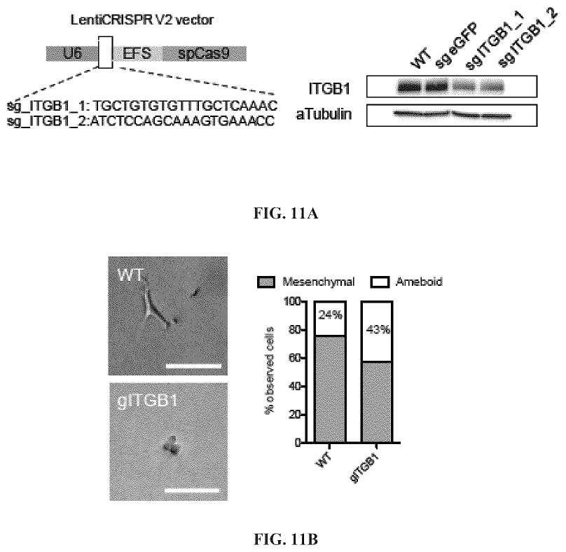

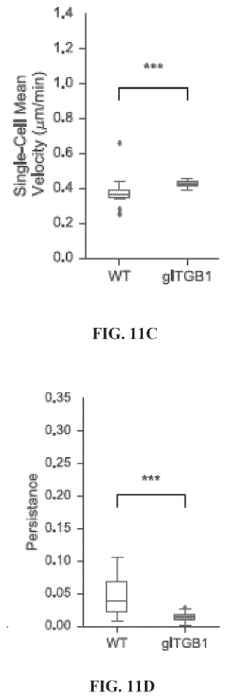

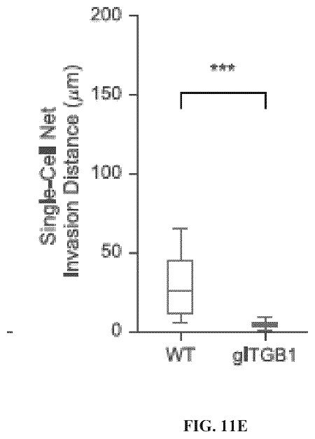

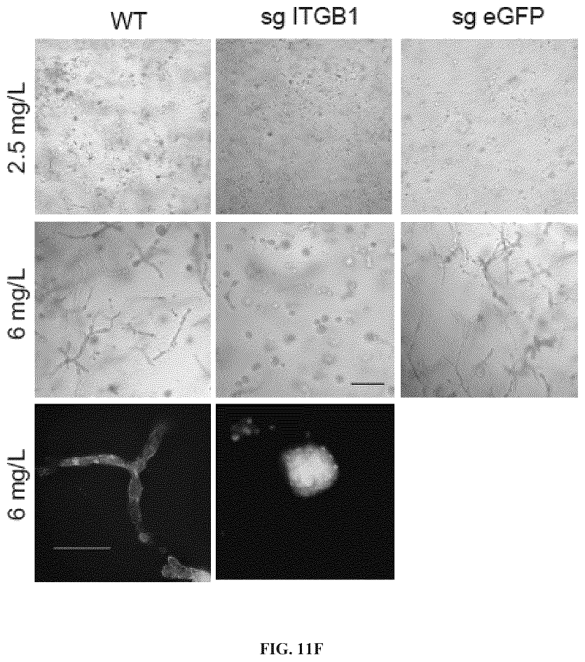

Since matrix stiffness has been implicated in driving epithelial to mesenchymal transitions (EMT) and aggressive phenotypes, it was asked whether increased stiffness of the high density collagen matrix was responsible for triggering transdifferentiation. To test this, a collagen polymerization procedure was developed that increases the stiffness of the low density matrix to match the stiffness of the high density matrix (FIG. 10A). By lowering the polymerization temperature from 37 °C to 20 °C, polymerization slowed, allowing fibers to form more organized and reinforced fiber structures with larger pores (FIG. 13I). Breast cancer cells cultured in this stiffened low density condition did not undergo network formation (FIG. 10B), suggesting that 3D stiffness is not sufficient for triggering the transdifferentiation.

Determining whether the smaller pore size of the high density matrices triggered transdifferentiation. One way in which smaller pore sizes could influence cell behavior is by restricting the diffusion of molecules to and from the cells. More specifically, the imbalance between oxygen diffusion to cells and oxygen consumption by cells in 3D matrices has been shown to promote hypoxic conditions in some cases. Since regions of VM have previously been associated with markers of hypoxia in vivo, without being bound by theory, it is believed that cells in high density collagen created a more hypoxic condition than in low density collagen and that low oxygen levels could trigger network formation. To test this, MDA-MB-231 cells were cultured in low density collagen under a hypoxic atmosphere of 1% oxygen for one week. To confirm that a hypoxic response was achieved, the level of HIF1A mRNA expression by RT-qPCR at day seven was assessed and revealed a significant decrease in HIF1A expression (FIG. 10C). This is a common response to long-term hypoxia by various cancer cell lines. However, hypoxia was not sufficient to induce network formation in any portion of the cancer cell population in the low density collagen matrix (FIG. 10D, left). For comparison, the HIF1A mRNA expression of breast cancer cells cultured for one week in low density collagen under 21% oxygen, in high density collagen under 1% oxygen, and in high density collagen under 21% oxygen was also assessed (FIG. 10C). Without being bound by theory, these results support the conclusion that cells cultured in high density collagen experience increased hypoxia compared to cells cultured in low density collagen under normal atmospheric conditions. Nevertheless, the hypoxic response achieved in low density collagen under 1% oxygen exceeded that induced by high density matrix alone. Cells in high density matrix under 1% oxygen continued to predominately display a network phenotype (FIG. 10D, right), but the average network length (FIG. 10E) was significantly shorter than cells in high density collagen under normoxic conditions (FIG. 13H). Previous studies have reported that hypoxia is not sufficient to induce a VM phenotype in melanoma cells in vitro. Without being bound by theory, it is possible that in vivo, additional stromal cell secreted factors or cell-cell interactions modulated by hypoxia may indirectly influence the VM process.

To further explore whether pore size reduction induced transdifferentiation of cancer cells, this parameter was interrogated independently of collagen density. In this model, the high density condition contains 2.4 times more collagen than the low density condition. This increase in total collagen reduces pore size, but also presents more adhesive ligands to cells, which could increase integrin activation. To separate pore size from bulk density, a collagen structure engineering technique was developed that reduced the pore size and fiber length of the low density matrix to approximate that of the high density matrix. Under normal polymerization conditions, low density collagen self-assembles into relatively long, structured fibers. When non-functionalized, inert polyethylene glycol (PEG) was mixed into collagen monomer solution prior to polymerization, molecular crowding restricted fiber formation. This resulted in shorter, more interconnected fibers yielding smaller pores (FIGS. 10F-I) without increasing stiffness (FIG. 10A). Breast cancer cells encapsulated in this pore-size-reduced low density matrix underwent network formation over the course of one week (FIG. 10J). To control for the possible influence of PEG itself, PEG was added into media on top of a normally polymerized low density gel embedded with cells and allowed to diffuse into the interstitial spaces among the fibers to reach the same final concentration as was used in the pore-size-reduced low density matrix (10 mg mL⁻¹ PEG). Cells maintained in this molecularly crowded condition over one week did not form networks, but instead remained as single cells. However, a noticeable slowing of cell migration occurred, which resulted in an anisotropic patterning of single cells throughout the matrix (FIG. 10K). These results suggested that the fiber architecture of high density collagen induces network formation independently of the bulk increase in adhesive ligand density and confirms that bulk matrix stiffness is not involved.

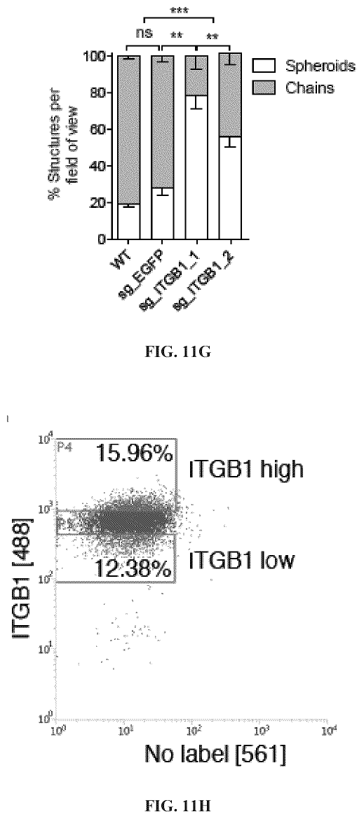

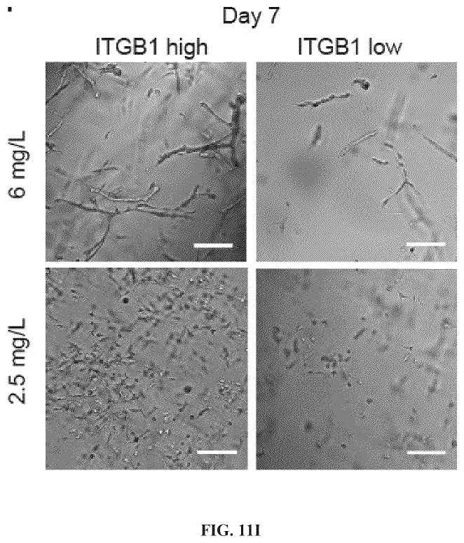

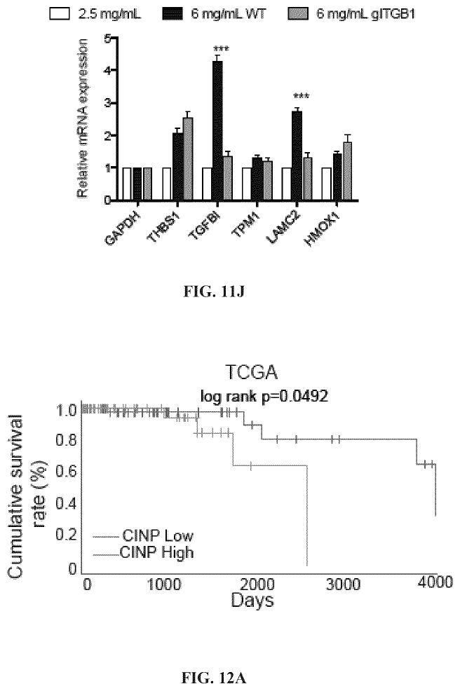

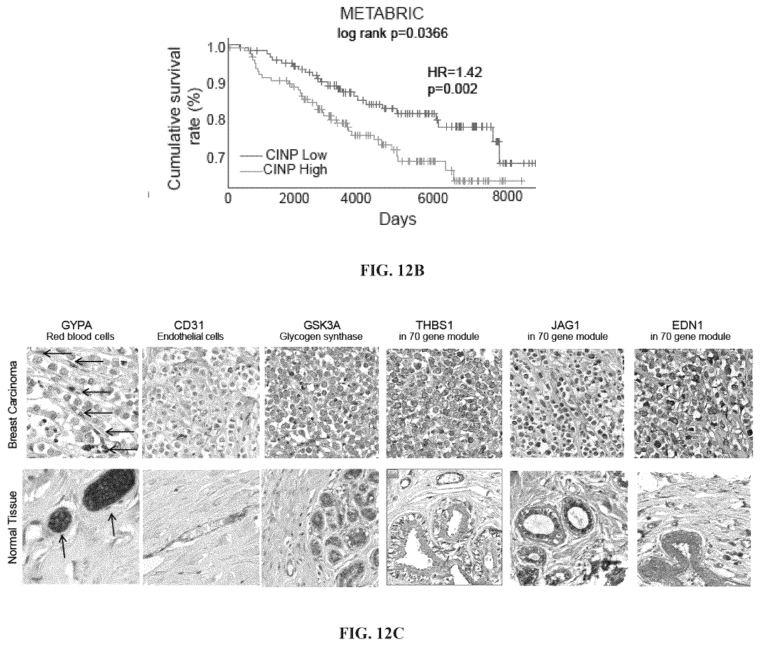

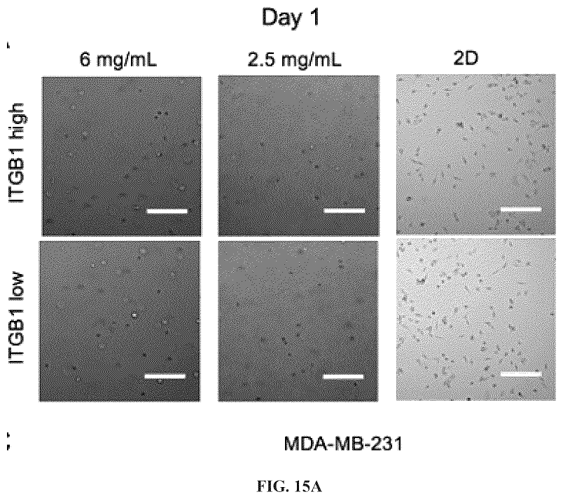

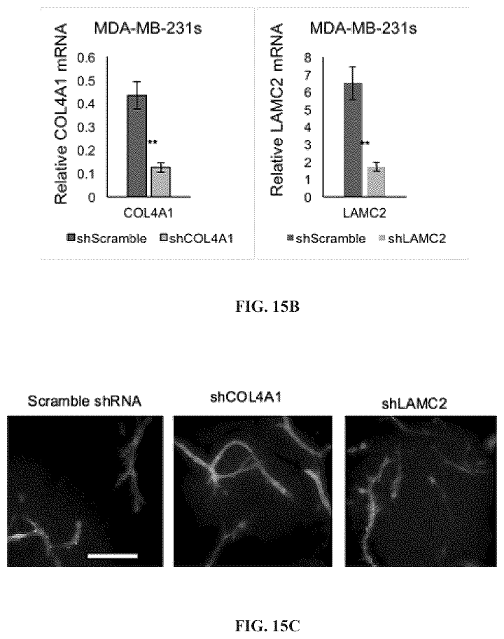

The short, more isotropic arrangement of fibers associated with both the high density collagen and low density PEG crowded collagen conditions could act on cells through local cell-matrix interactions transduced by integrin signaling. Integrin β1 (ITGB1) is a canonical receptor for collagen I, a central node in ECM signal transduction, and a critical mediator of breast cancer progression in mouse and in vitro models. Here, ITGB1 was upregulated by both cancer cell types in response to confining matrix conditions (FIG. 9B). Thus, it was next asked whether the network forming phenotype observed in confining matrix conditions was mediated by ITGB1. CRISPR-Cas9 technology was used to silence ITGB1 expression with single guide RNAs (sgRNAs), and constructs expressing sgRNAs targeting eGFP were used as controls (FIG. 11A). Silenced and control cells were embedded separately and sparsely in low and high density collagen matrices. Cells were monitored by timelapse microscopy for early migration behavior then imaged again after one week. In low density collagen, ITGB1 silenced cells maintained a similar level of migration capability to WT cells in low density matrices, but used an amoeboid blebbing migration phenotype instead of a mesenchymal migration phenotype (FIG. 11B). In high density conditions, ITGB1 silenced cells migrated faster than WT cells, but were significantly less persistent and did not invade (FIGS. 11C-E). Surprisingly, after one week ITGB1-silenced cells in high density collagen formed spheroid structures instead of cell networks, whereas control cells exhibited the same behavior as the wild type in both collagen conditions (FIG. 11F). Retrospective analysis of WT MDA-MB-231 cells in high density collagen revealed that a small fraction spontaneously formed spheroid structures (FIG. 11G). These findings suggest that either basal expression level or upregulation of ITGB1 dictates the network forming phenotype. To distinguish between these two possibilities, the parental WT population was sorted based on basal ITGB1 expression level and then embedded high and low expressing cells separately in confining high density collagen matrices (FIG. 11H). No appreciable differences were observed in the percentage of networks versus spheroids formed by the sorted populations after one week. However, ITGB1 low cells proliferated less and displayed fewer total number of network or spheroid structures (FIG. 11I, and data not shown) even though the initial seeding density was the same (FIG. 15A).

To further explore the link between the upregulated transcriptional module and the network forming phenotype, we asked whether ITGB1 silenced spheroid forming cells showed different gene expression patterns than WT network forming cells. To assess this, qRT-PCR analysis was conducted of a subset of the 70-gene panel in the two cell phenotypes. Upregulation of several key genes were maintained in the spheroid forming cells, while other genes were no longer upregulated (FIG. 11J). These results show that ITGB1 regulates some aspects of the transcriptional module associated with the network forming phenotype.

Finally, it was asked if upregulated genes in the transcriptional module that have previously been implicated as drivers of VM in vitro were functionally active in the network forming phenotype. LAMC2 (Ln-5, gamma 2 chain) was previously found to be upregulated in aggressive melanoma cells that intrinsically display the VM phenotype compared to less aggressive melanoma cells that don't display VM. Moreover, it was implicated as a driver of VM network formation, since the cleavage of this secreted matrix molecule by MMP-2 and MT1-MMP produces pro-migratory fragments. In 2D culture of aggressive melanoma cells on top of collagen I, the inhibition of LAMC2 cleavage blocked VM network formation. Using shRNA to knock down LAMC2, we found that LAMC2 KD MDA-MB-231 cells maintain their ability to form network structures in 3D high density collagen (FIGS. 15 B-C). COL4A1 is another matrix molecule upregulated by cells undergoing the network phenotype (FIG. 8H and FIG. 9G) and previously implicated in driving migration. COL4A1 KD in MDA-MB-231 cells also did not inhibit the ability of cells to form network structures in 3D high density collagen (FIGS. 15B-C).

### CINP Transcriptional Module Predicts Poor Prognosis in Human Cancer

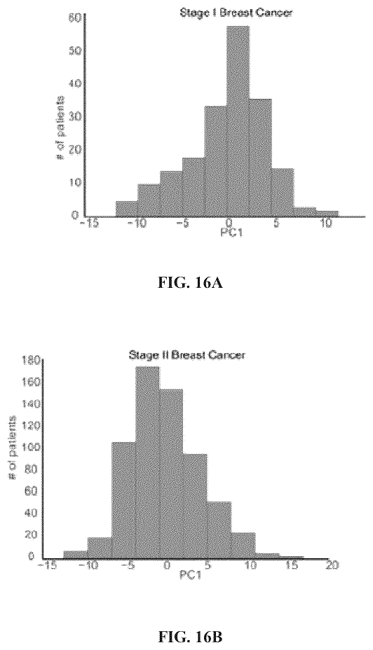

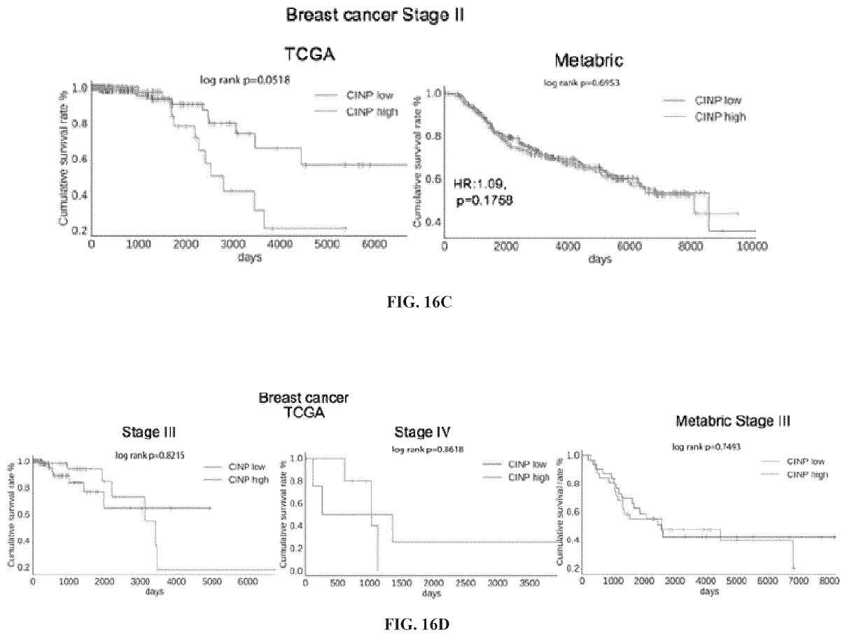

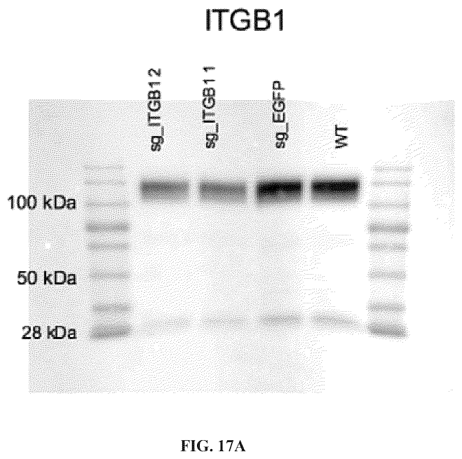

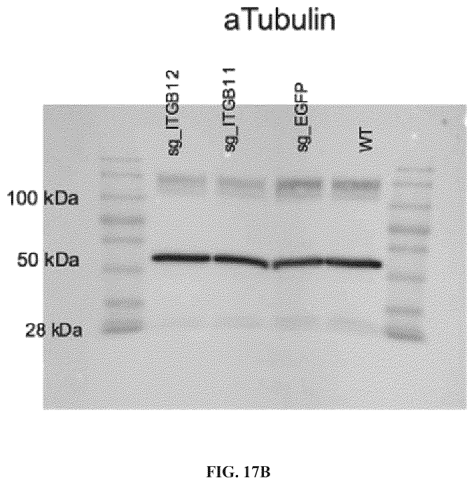

Finally, to determine if the CINP triggered by the 3D system was clinically relevant, analysis was performed to determine whether the 70 common-to-cancer genes associated with the CINP could predict cancer patient prognosis. Without being bound by theory, it was anticipated that if this gene signature was indicative of a more metastatic cancer cell migration phenotype, its expression would correlate with poor patient outcomes. Since late stage tumors are already characterized by migration of tumor cells to distant lymph nodes or organs, without being bound by theory, it was hypothesized that a gene signature associated with metastatic migration would correlate with prognosis in early (Stage I & II) but not late (Stage III & IV) stage tumors. Using the cancer genome atlas (TCGA), data was analyzed for breast cancer patients with respect to the expression of the 70 gene signature. An expression metagene was constructed using the loadings of the first principal component (CINP PC1) of a 195 Stage I patient by 70 gene matrix (FIG. 16A, also see methods). Then a survival analysis was conducted, comparing patients with the highest (top 30%) and lowest (bottom 30%) expression metagene scores by log rank test. The cumulative survival rate of these two groups differed significantly (log rank p=0.049); however, there was insufficient data to power a hazard ratio (HR) calculation (FIG. 12A). Analysis using the more data-rich METABRIC microarray database of breast cancer patients showed similar results for Stage I, confirming the prognostic value of the gene set (log-rank p=0.037, HR=1.40, Cox p=0.002, FIG. 12B). Applying the same analysis to Stage II breast cancer patients revealed that the CINP metagene was associated with a marginally significant difference in 5-year survival by TCGA analysis but not by METABRIC analysis (FIGS. 16B-C). One caveat to this analysis is that data for 11 of the genes in the 70 gene panel were not available in the METABRIC dataset. The CINP metagene also did not separate patients with better prognosis in late stage tumors (FIG. 16D). These results indicate that the CINP gene module could have clinical predictive power in the early stages of breast cancer. Importantly, further analysis of Stage I patients by molecular subtype revealed that the CINP metagene provided significant prognostic value for Luminal A and Triple Negative breast cancer patients (Table 6).

TABLE 6
CINP score potential to predict prognosis in Stage I patients
from metabric database broken down by molecular subtype.

| Metabric molecular | Patient | Death | | |
|---|---|---|---|---|
| subtype | count | Observed | HR | Cox p |
| Luminal B | 126 | 33 | 1.2461 | 0.3194 |
| Luminal A | 202 | 34 | 1.5996 | 0.0162 |
| Triple Negative | 63 | 14 | 3.8537 | 0.0070 |
| HER2+ | 39 | 13 | 0.7152 | 0.3405 |

Analysis of CINP score potential to predict prognosis in Stage I patients from METABRIC database broken down by molecular subtype.

Next, the predictive value of the gene module in additional cancer types in TCGA independently of stage or subtype was screened using only age and CINP score as covariates. The CINP gene module was a significant predictor of survival in lung adenocarcinoma (LUAD), lower grade glioma (LGG), cervical squamous cell carcinoma and endocervical adenocarcinoma (CESC), pancreatic adenocarcinoma (PAAD), mesothelioma (MESO), adrenocortical carcinoma (ACC), bladder urothelial carcinoma (BLCA), and kidney chromophobe carcinoma (KICH) (Table 7), but was not a significant predictor in several other tumor types found in TCGA.

TABLE 7
TCGA Pan cancer analysis independent of Stage

| Cancer type | Patient count | Death Observed | HR | Cox p |
|---|---|---|---|---|
| LGG | 508 | 92 | 1.8434 | 1.1E−13 |
| ACC | 79 | 25 | 3.1863 | 2.8E−04 |
| CESC | 304 | 60 | 1.6560 | 5.2E−04 |
| MESO | 85 | 28 | 1.6101 | 6.9E−04 |
| PAAD | 178 | 59 | 1.5948 | 2.2E−03 |
| BLCA | 409 | 111 | 1.3338 | 0.0053 |
| LUAD | 521 | 124 | 1.2448 | 0.0169 |
| KICH | 64 | 8 | 2.9277 | 0.0210 |

Table showing results from Kaplan Meier and hazard ratio analysis across all cancer types in TCGA where the CINP gene score is significant predictor of prognosis (p<0.05).

Finally, it was determined whether the in vitro network forming phenotype and associated transcriptional signature were related to the clinical VM phenotype. Using the Human Protein Atlas (www.proteinatlas.org), breast cancer tumor slices displaying hallmarks of the VM phenotype were identified, namely linear chains of cells lining glycogen-rich matrix networks that conduct blood flow but do not stain positively for CD31. The tumor of patient 1910 displayed linear chains of cancer cells lining interconnected matrix networks (FIG. 12C). An immunohistochemical stain for GYPA showed red blood cells flowing through the matrix-networks in tumor tissue but highly concentrated in vessel-like structures in healthy tissue. A stain against CD31 showed that there were no endothelial cells lining the matrix networks in the tumor tissues. Although a PAS stain was not available in the protein atlas database, which would determine whether the matrix networks were positive for glycogen, a stain against glycogen synthase (GSK3A) was available and showed that the chains of cancer cells significantly expressed this enzyme. The network forming cell phenotype combined with IHC evidence are consistent with the previously described histopathology of VM. Next, it was asked whether highly upregulated genes in the 70 gene CINP module were evident at the protein level in this clinical sample of VM. Stains for THBS1, JAG1, and EDN1 were available in the protein atlas database for the same tumor and showed significant expression of all three genes from the CINP transcriptional module in the VM tumor tissue but little stain in healthy tissues.

The transcriptional, histopathologic, and phenotypic data suggest that the in vitro CINP and clinical VM share many commonalities. This is the first time that collagen fiber architecture, characterized by short fibers and small pores, has been identified as an inducer of cancer transdifferentiation associated with a VM-like phenotype or more normal acinar phenotype, depending on the capacity of cells to upregulate ITGB1. More broadly, these findings show that collagen fiber architecture modulates the role ITGB1 plays in migration. In one architectural context, ITGB1 facilitates a switch from mesenchymal to amoeboid migration and in another architectural context it mediates migration persistence and the shape of structures formed by collective morphogenesis.

Although ITGB1 was critical for directing the fate of cells during collagen induced transdifferentiation, it was not necessary for initiating the transition from single cell to collective morphogenesis. Without being bound by theory, the response appears to be unique to stem-like cancer cells (MDA-MB-231 and HT1080) as opposed to normal cells (HFF-1). Since, in this system, cells are embedded sparsely and undergo transcriptional reprogramming prior to cell division, the involvement of cell-cell interactions does not appear to play a role in transdifferentiation initiation. Without being bound by theory, it is possible that cell interactions with the unique matrix architecture involve matrix sequestration of soluble factors and autocrine signaling. Indeed, TGF pathways were implicated by GO enrichment analysis (FIG. 9F). Alternatively, the initial confinement and rounded geometry of the cells enforced by the matrix may play a role. Several studies support a role for cellular geometry in numerous cellular processes including gene expression and differentiation, some of which is mediated by RhoA and cytoskeletal tension. However, confinement in Matrigel did not trigger the same process, indicating a unique requirement for cell-collagen interaction. Future work will address these questions.

ECM molecules COL4A1 and LAMC2 were also upregulated by CINP cells and have previously been implicated in driving migration and VM network formation in 2D culture. In this 3D collagen system, knockdown of either gene was not sufficient to block the VM-like phenotype (FIG. 15). This suggests that regulation of in vitro cell network formation in a more physiological 3D culture context is distinct from regulation in a 2D culture context, which has implications for understanding molecular mechanisms. Given the significantly different requirements for cell movement in 3D ECM, such as matrix degradation and remodeling, this study highlights the importance of both the type of matrix and the dimensional context for studying physiological migration strategies.

Interestingly, SERPINE1, a secreted protease inhibitor involved in coagulation and inflammation regulation, was upregulated by cancer cells as well as normal fibroblasts in response to confining collagen architectures. Cells which intrinsically expressed SERPINE family members were most efficient at spreading hematogenously, a characteristic that also correlated with their capacity to undergo VM in vivo. Without being bound by theory, both cell-intrinsic and ECM factors may contribute to the emergence of VM. Interestingly, the finding that fibroblasts and cancer cells both upregulate SERPINE1 expression in confining collagen conditions supports a role for stromal cells in SERPINE mediated VM metastasis.

The significant predictive value of the CINP gene signature in several tumor types may signify the physiological relevance of the ECM context and network forming migration phenotype created in vitro to a conserved mechanism of solid tumor metastasis. Without being bound by theory, it is possible that gene expression analysis of additional cancer cell types induced into VM-like behavior by the 3D collagen system could help to further refine the conserved CINP gene module. Without being bound by theory, this would facilitate prioritization of the genes for targeted functional studies to identify key regulators and potential therapeutic targets. In addition to regulators of the CINP, the conserved gene module also likely contains elements responsive to collagen but not directly involved.

Profiling additional cancer cell types and patient derived tumor cells could also help to refine the gene module's prognostic value in the nine tumor types already identified or define additional cancer specific versions of the CINP. Validation of the prognostic value of this gene module could help patients avoid the long-term side effects of aggressive radiation and chemotherapy if the likelihood of metastasis is very low. Without being bound by theory, molecular detection of VM markers could provide a more quantitative measure.

### Example 3 Methods

Cell culture. HT-1080 and HFF-1 were purchased from (ATCC, Manassas, VA) MDA-MB-231 cells were provided by Adam Engler (UCSD Bioengineering). All cell lines were cultured in high glucose Dulbecco's modified Eagle's medium supplemented with 10% (v/v) fetal bovine serum (FBS, Corning, Corning, NY) and 0.1% gentamicin (Gibco Thermofisher, Waltham, MA) and maintained at 37 °C and 5% CO₂ in a humidified environment during culture and imaging. The cells were passaged every 2-3 days. Cell culture under hypoxia was done on a humidified and temperature controlled environment at 1% O₂. Cells were tested for mycoplasma contamination using the Mycoalert kit (Lonza, Basel, Switzerland) before performing experiments.

3D culture in collagen I matrix. Cells embedded in 3D collagen matrices were prepared by mixing cells suspended in culture medium and 10× reconstitution buffer, 1:1 (v/v), with soluble rat tail type I collagen in acetic acid (Corning, Corning, NY) to achieve the desired final concentration^{10,20,21}. 1 M NaOH was used to normalize pH in a volume proportional to collagen required at each tested concentration (pH 7.0, 10-20 μl 1 M NaOH), and the mixture was placed in 48 well culture plates and let polymerize at 37 °C. Final gel volumes were 200 μL.

Cell tracking and motility analysis. Cells were embedded in 3D collagen matrices in 48 well plates and left polymerize for 1 hour in a standard tissue culture incubator and then 200 μL of complete growth medium were added on top of the gels. The gels were transferred to a microscope stage top incubator and cells were imaged at low magnification (×10) every 2 minutes for 48 h. Coordinates of the cell location at each time frame were determined by tracking single cells using image recognition software (Metamorph/Metavue, Molecular Devices, Sunnyvale, CA). Tracking data was processed using custom written python scripts based on previously published scripts to calculate cell speed, invasion distances and Mean Squared Displacements (MSDs). For cell motility analysis before and after division the time lapse videos were scanned to identify dividing cells within the imaging period and the division point was identified as the frame at which a clear separation could be identified between daughter cells. The dividing cell was tracked up to the division point and one of the daughter cells (randomly chosen) was tracked from that point until the 48 h time point. For collective cell invasion distance the 48 h time lapse video was processed to obtain the maximum intensity projection (MIP), which highlights the tracks taken by the cells/groups of cells. Individual tracks distinguishable in the MIP were measured to obtain an equivalent invasion distance. All cell tracking data comes from 3 independent experiments performed on different days and with different cell passages.

Persistence random walk model implementation. To quantify the differences in the mean squared displacement (MSDs) the MSDs were fitted for each condition using the persistent random walk model (PRW model) as described in^{53,54}. Briefly, the MSDs were calculated as in Equation 1. The Equation 2 describing the PWR was fitted using python's Imfit library for each MSD. The persistent time (parameter P) was then extracted to calculate differences between groups as presented in FIG. 1A-B. DMPSID=1

Where x and y are que coordinates of the position of a cell at each time point and tau is the time lag.

Where, S is the cell speed and P is the persistence time and o is a function of the error in the position of the cell as described in.

Collagen stiffness modification and measurement using shear rheology. To modify the stiffness of collagen matrices without increasing density of material, kept 2.5 mg mL⁻¹ gels at 20 °C for 30 minutes until they were fully polymerized. After the initial polymerization the gels were placed on a humidified tissue culture incubator at 37 °C for at least 1 hour extra before adding cell growth media on top. To measure the effect of polymerization temperature on the gel stiffness recreated the polymerization conditions for rheology testing (hybrid rheometer (DHR-2) from TA Instruments, New Castle, DE) using a cone and plate geometry with a sample volume of 0.6 mL. Shear storage modulus G′ was measured as reported before¹⁰. Briefly, first performed a strain sweep was from 0.1% to 100% strain at a frequency of 1 rad/s to determine the elastic region. Then a frequency sweep was performed at a strain within the linear region (0.8%) between 0.1-100 rad/s. Three independent replicates were performed for each condition tested.

Collagen structure modification using Poly-Ethylene-Glycol. To modify the structure of the collagen fibers within the gels without changing the final collagen concentration, Polyethylene glycol (PEG, MW=8000, Sigma, St. Louis, MO) was solubilized in phosphate-buffered solution (PBS), filter sterilized. Solubilized PEG was then mixed into the cells, reconstitution buffer solution described above to produce a final PEG concentration of 10 mg/mL in the collagen gel. The gels were allowed to polymerized in the same conditions as collagen only gels. Collagen structure modification was verified using confocal reflection microscopy.

RNA Isolation and purification. 3D collagen I gels were seeded in three independent experiments and harvested after 24 hours of culture for RNA extraction and directly homogenized in Trizol reagent (Thermofisher, Waltham, MA). Total RNA was isolated following manufacturer's instructions. Isolated RNA was further purified using High Pure RNA Isolation Kit (ROCHE, Branford, CT). RNA integrity was verified using RNA Analysis Screen Tape (Agilent Technologies, La Jolla, CA) before sequencing.

RNA sequencing and data analysis. Biological triplicates of total RNA were prepared for sequencing using the TruSeq Stranded mRNA Sample Prep Kit (Illumina, San Diego, CA) and sequenced on the Illumina MiSeq platform at a depth of >25 million reads per sample. The read aligner Bowtie2 was used to build an index of the reference human genome hg19 UCSC and transcriptome. Paired-end reads were aligned to this index using Bowtie2 and streamed to eXpress for transcript abundance quantification using command line “bowtie2 −a −p 10 −x/hg19 −1 reads_R1.fastq −2 reads_R2.fastq|express transcripts_hg19.fasta”. For downstream analysis TPM was used as a measure of gene expression. A gene was considered detected if it had mean TPM>5.

Gene ontology term overrepresentation analysis. To assess the overrepresented GO terms the cytoscape app BiNGO was used. Statistical test used was hypergeometric test, Benjamini-Hochberg false discovery rate (FDR) correction was used to account for multiple tests and the significance level was set at 0.05. For a given term, to assess the sensitivity of the enriched gene sets to the genes used in the analysis, varied the threshold for including a gene as differentially upregulated from a fold change of 1.3 to a fold change of 1.9. The probability of a gene enriched with term is (# of genes in background with term)/(# of genes in background). The fold enrichment is the observed number of genes associated with term divided by the expected number of genes associated with term.

Gene expression using qPCR. For qPCR experiments RNA was extracted as stated above and cDNA was synthesized using superscript iii first-strand synthesis system (Thermofisher, Waltham, MA). Relative mRNA levels were quantified using predesigned TaqMan gene expression assays (Thermofisher, Waltham, MA). Relative expression was calculated using the DCt method using GAPDH as reference gene. Assays used were: GAPDH (Hs02758991_g1), HIF1A (Hs00153153_m1), THBS1 (Hs00962908_m1), TGFBI (Hs00932747_m1), TPM1 (Hs04398572_m1), LAMC2 (Hs01043717_m1), HMOX1 (Hs01110250_m1).

Immunofluorescence and cell imaging. For cell imaging after 7 days of culture to visualize VM structures collagen gels were fixed using 2 washes of 4% PFA for 30 mins each at room temperature. F-actin was stained using Alexa Fluor® 488 Phalloidin (Cell signaling technology, Danver, MA) and the nuclei were counterstained with DAPI. For immunofluorescence staining the gels were incubated with the primary antibody for 48 to 72 hours. Anti-COL4A1 (1:200 dilution, NB120-6586, novus biologicals).

Confocal reflection imaging and quantification: Confocal reflection images were acquired using a Leica SP5 confocal microscope (Buffalo Grove, IL) equipped with a HCX APO L 20×1.0 water immersion objective. The sample was excited at 488 nm and reflected light was collected without an emission filter. For the estimation of pore size, used modification of a previously reported digital imaging processing technique. Briefly, the images were normalized to account for uneven illumination effects. Then a threshold was applied to generate a binary mask where pores were identified as the darkest areas of the image. Pore diameter was measured using NIS elements software (Nikon Instruments Inc., Melville, NY) measure objects tool.

Gene Suppression: The lentiCRISPR v2 was a gift from Feng Zhang (Addgene plasmid #52961). Small guide RNAs were cloned targeting the genes of interest into the lentiCRISPR v2 following Zhang's lab instructions. The sg_RNA sequences using were taken from the GECKO human library A. Used sequences were: ITGB1 sg_RNA1 (5′-TGCTGTGTGTTTGCTCAAAC-3′) (SEQ ID NO.: 1), ITGB1 sg_RNA2 (5′-ATCTCCAGCAAAGTGAAACC-3′)) (SEQ ID NO.: 2), EGFP sgRNA (5′-GGGCGAGGAGCTGTTCACCG-3′)) (SEQ ID NO.: 3). The lentiCRISPR v2 vectors with the cloned desired sgRNA were sequence verified and viral particles were generated by transfecting into lentiX293T cells (Clonetech, Mountain View, CA. Cat #632180) along with packaging expressing plasmid (psPAX2, Addgene #12260) and envelope expressing plasmid (pMD2.G, Addgene #12259). Viral particles were collected at 48 h after transfection and they were purified by filtering through a 0.45 μm filter. Target cells were transduced with the viral particles in the presence of polybrene (Allele Biotechnology, San Diego, CA). After overnight incubation media was changed and cells were left 24 h-48 h in normal growth media and then changed to puromycin selection media (2.5 μg/mL puromycin) for 7 days before experiments were performed. For shRNA mediated gene knock down, Glycerol stocks of TRC2-pLKO.1-puro shRNA targeting LAMC2 (NM_005562.1-1019s1c1: CCGGGCTCACCAAGACTTACACATTCTCGAGAATGTGTAAGTCTTGGTGAGCTTTTTG) (SEQ ID NO.: 4), COL4A1 (NM_001845.3-3859slc1: CCGGCCTGGGATTGATGGAGTTAAACTCGAGTTTAACTCCATCAA TCCCAGGTTTTTG) (SEQ ID NO.: 5) and a non-targeting scramble sequence (SHC016: CCGGGCGCGATAGCGCTAATAATT TCTCGAGAAATTATTAGCGCTATCGCGCTTTTT) (SEQ ID NO.: 6) were purchased from Sigma-Aldrich packaged in LentiX293T (Clonetech, Mountain View, CA. Cat #632180) along with packaging expressing plasmid as described above. Lentiviral particles were transduced into target cells and stably expressing cells were selected with puromycin (2 ug/mL) for at least 5 days before using.

Western blotting: Cells were grown to >90% confluency in 100 mm dishes. After washing 2× with PBS cells were collected into 100 μL of lysis buffer with 1× Halt protease inhibitor cocktail (Pierce IP lysis Buffer, Thermofisher, Waltham, MA) by thoroughly scraping the dish surface. Cell lysate was incubate in ice with constant shaking for 30 min and then centrifuged at 15,000×g for 20 for protein purification. Samples were loaded at 50 ug total protein concentration for SDS-PAGE. Membranes were probed with antibodies against ITGB1 (#4706 from Cell signaling technology, Danver, MA. 1:10000 dilution) and Tubulin (TU-01 MA1-19162, Thermofisher, Waltham, MA. 1:30000 dilution).

Fluorescence activated cell sorting (FACS): Wild type MDA-MB-231 cells were grown in collagen I coated tissue culture dished until 80% confluence. Cells were harvested using HyClone HyQtase (GE Healthcare Life Sciences, Marlborough, MA) and resuspended in FACS buffer (1% BSA, 0.5 mM EDTA in PBS). The cell suspension was then labeled using a monoclonal antibody against human CD29 (b1 integrin) conjugated to AlexaFluor 488. A cell suspension without added antibody was used as negative control. After labeling, the cells were analyzed within 1 hour of detachment at the stem cell core of Sanford Consortium of Regenerative Medicine (La Jolla, CA) using a BD Influx cell sorter (BD, Franklin lakes, NJ). Cells were sorted based on fluorescence intensity into the top expressing population (~15%, ITGB1 high) and bottom expressing population (~13%, ITGB1 low). Sorted cells were replated into collagen coated dishes and left to recover overnight. After recovery the cells were embedded in 3D collagen gels as described above.

Experimental data analysis and statistics: All cell motility data was analyzed for statistical significance using the scipy python package. Additional experimental data was analyzed using prism graphpad (San Diego, CA). Significance (p) was indicated within the figures using the following scale: * p<0.05 ** p<0.01 *** p<0.001. Additional relevant information is detailed in the figure captions.

TCGA data reprocessing and survival analysis: The TCGA raw data were downloaded from CGHub directly using gtdownload. Corresponding clinical metadata were obtained from the TCGA data portal (https://tcga-data.nci.nih.gov/docs/publications/tcga/). RNAseq fastq files were realigned and quantified using sailfish v.0.7.6 with default parameters. Only primary tumors were considered in the analysis. In the analysis of breast invasive carcinoma, only the patients with reported histological staining for the three markers (Her2, ER, PR) could be associated with a molecular subtype. Patients for which any of the histological markers were not evaluated or were detected at an equivocal level were assigned to an “unknown” subtype. TCGA data for Stage I, II, III and IV breast cancer patients was analyzed by Principal Component Analysis (PCA) with respect to the 70 CINP genes to construct gene expression meta-markers as previously described. PCA-based score quantiles were mapped to CINP high and CINP low categories based on mean CINP gene expression levels. Because the CINP signature comprised only genes that were upregulated in the presence of the network phenotype, the overall mean expression of CINP genes was used to map PCA score to CINP signature activity level.

METABRIC data retrieval and survival analysis. The clinical and microarray expression dataset was from cBioPortal (www.cbioportal.org/study?id=brca_metabric). 59 out of 70 CINP genes mapped to METABRIC microarray data (missing genes: ZNF532, TRMT13, AMIGO2, KIN, NKX3-1, TANC2, TVP23C, SDHAP1, MTND2P28, GTF2IP4, H2BFS). Survival analysis was performed using the same method as described above for TCGA data. The Cox multiple regression uses CINP score, age, and three molecular subtype categories as covariates.

TCGA pan cancer analysis. Tumor types for which at least 100 patients had both expression and clinical metadata were analyzed to determine correlation between a CINP gene expression and 5-year survival. Only primary tumors were considered. Kaplan-Meter analysis was performed comparing the 30% of individuals with the lowest CINP expression score to the 30% with the highest score. The cox multiple regression uses age and CINP score as covariates. Both analyses use the Lifelines python library (lifelines.readthedocs.io/en/latest/). The log rank test was used to determine significance of survival differences between groups.

Human protein atlas data: The online database Human Protein Atlas was used to identify breast cancer tumor slices displaying hallmarks of the VM phenotype and subsequently assess protein expression of the genes associated with the in vitro network forming phenotype. The tumor of patient ID 1910 was found to display linear chains of cancer cells lining interconnected matrix networks and had been stained for numerous other proteins of interest. Histological images shown in FIG. 5D can be found at www.proteinatlas.org by searching for the gene name in the breast cancer database and selecting patient ID 1910.

Data availability. All sequencing data from this study has been deposited in the National Center for Biotechnology Information Gene Expression Omnibus (GEO) and is accessible through the GEO Series accession number GSE101209. All other relevant data are available within the Article and Supplementary Files, or from the corresponding author upon request.

Code availability: Relevant scripts for the analysis of TCGA and METABRIC data are available at: github.com/brianyiktaktsui/Vascular_Mimicry.

### Example 4: 3D High Density Culture System

It is well established that the collagenous extracellular matrix surrounding solid tumors significantly influences the dissemination of cancer cells. However, the underlying mechanisms remain poorly understood, in part because of a lack of methods to progressively modulate collagen fiber topology in the presence of embedded cells. In this work, a technique is developed to tune the fiber architecture of cell-laden 3D collagen matrices using PEG as an inert molecular crowding agent. With this approach, it is demonstrated that fiber length and pore size can be modulated independently of bulk collagen density and stiffness. Using live cell imaging and quantitative analysis, matrices with long fibers are shown to induce cell elongation and single cell migration, while shorter fibers induce cell rounding, collective migration, and morphogenesis. Without being bound by theory, it is concluded that fiber architecture is an independent regulator of cancer cell phenotype and that cell shape and invasion strategy are functions of collagen fiber length.

Accumulating evidence suggests that matrix architecture is capable of modulating cell migration phenotype as profoundly as matrix stiffness. Largely, studies of matrix architecture have relied on micropatterned 2D surfaces and have focused on imparting contact guidance. Systematically controlling 3D ECM architecture remains a substantial challenge. Yet, it is now widely appreciated that cell behavior is distinct in native 3D ECM. Compared to 2D models, changes in the abundance, localization, and functional status of intracellular proteins have been documented in 3D culture. Thus, a major tradeoff exists between the physiological relevance of an ECM model system and the ability to tune and control specific physical features. It is currently impossible to decouple all of the architectural features of a 3D fibrillar protein network. Nonetheless, several studies have developed novel methods to produce highly aligned, anisotropic 3D collagen matrices, which impart both contact guidance and stiffness anisotropy. These methods include magnetic, mechanical, and cell force driven reorganization of collagen fibers as well as electrospinning. From these studies, matrix stiffness and alignment have been established as modulators of cell phenotype through mechanotransduction processes. However, the mechanisms by which matrix architecture may act independently on cells are not clear. This is due in part to the scarcity of in vitro experimental techniques that satisfy the need to modulate fiber characteristics independently of collagen density and stiffness while also allowing cells to be fully embedded in 3D.

Molecular crowding (MC) is one approach that can potentially achieve these goals. Crowding is a physiologically relevant phenomenon whereby high concentrations of macromolecules occupy the extracellular space and generate excluded volume effects. In the context of collagen polymerization, this results in alterations to the rates of nucleation and fiber growth.

Thus far, architectural engineering of 3D collagen hydrogels with MC has been investigated in cell-free conditions. Herein is established a crowding technique for cell-laden 3D collagen matrices using biologically inert PEG. Adjustments to the amount of PEG added during collagen assembly and cell embedding reliably tune fiber topography. The biophysical properties of the MC matrices are quantitatively evaluated as well as the morphological and migration response of cancer cells in the engineered constructs. Importantly, through control experiments it is shown that the influence of the crowding agent on cell morphology and migration behavior occurs only through the topographic alterations MC induces in the matrix. Finally, matrix architecture is demonstrated as a critical modulator of cancer cell phenotype independently of matrix stiffness or density.

Macromolecular Crowding with PEG Tunes Collagen Fiber Characteristics

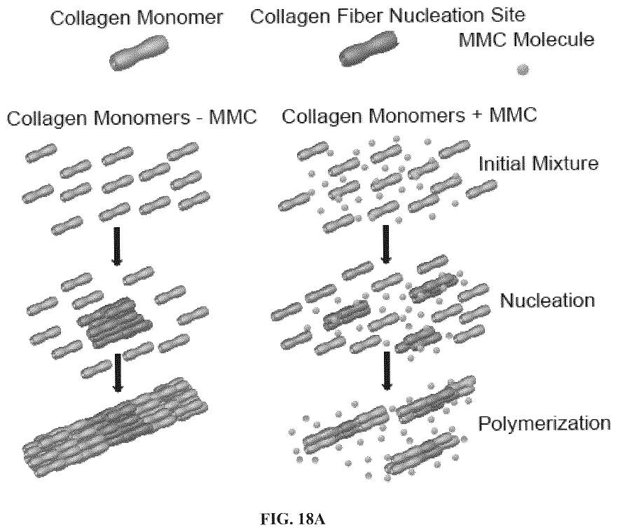

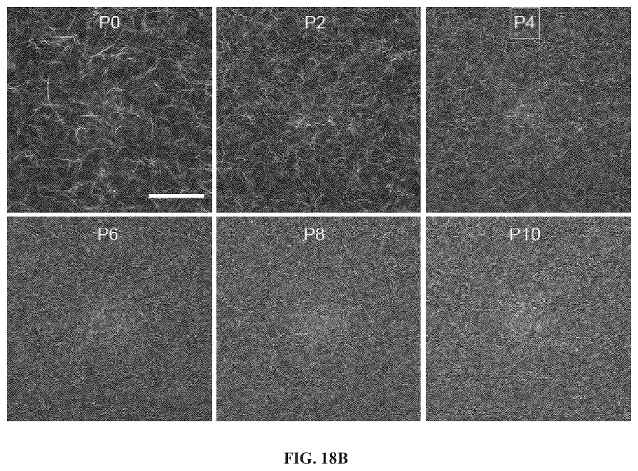

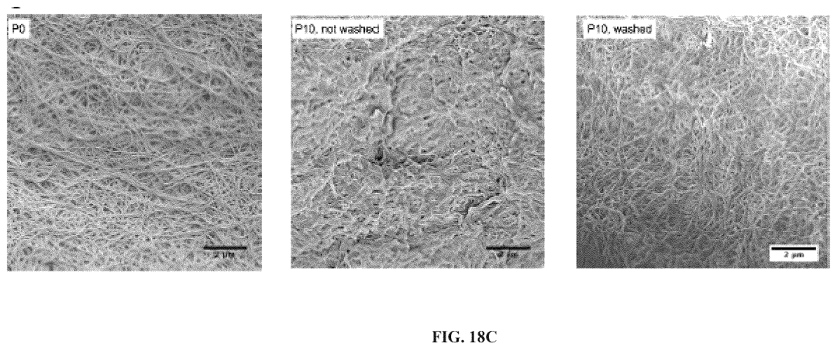

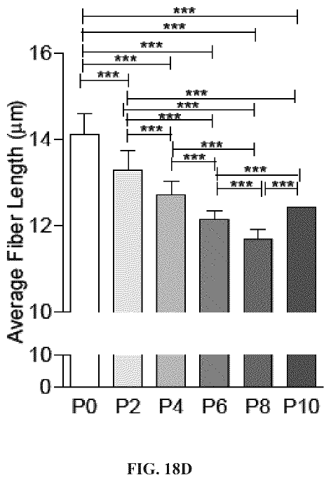

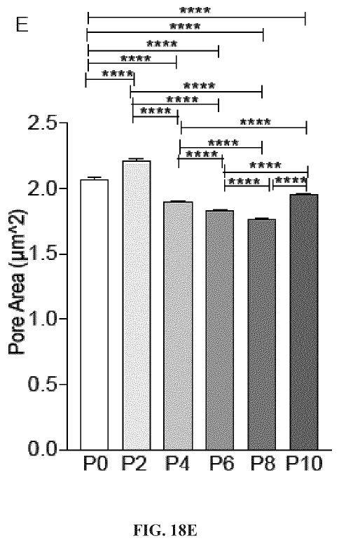

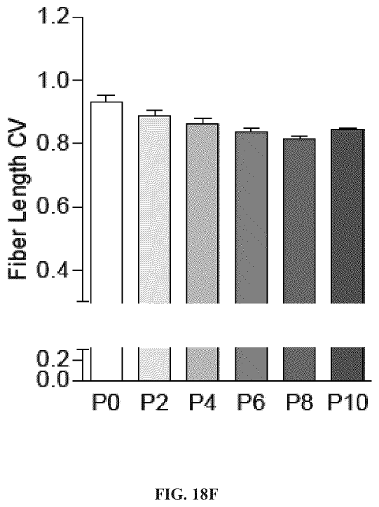

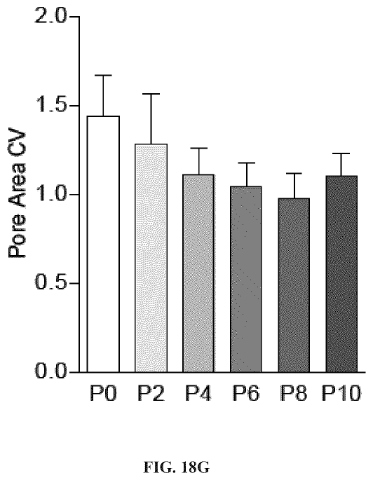

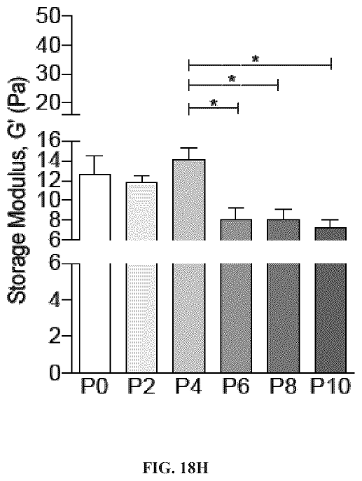

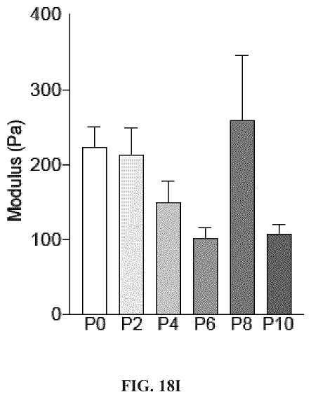

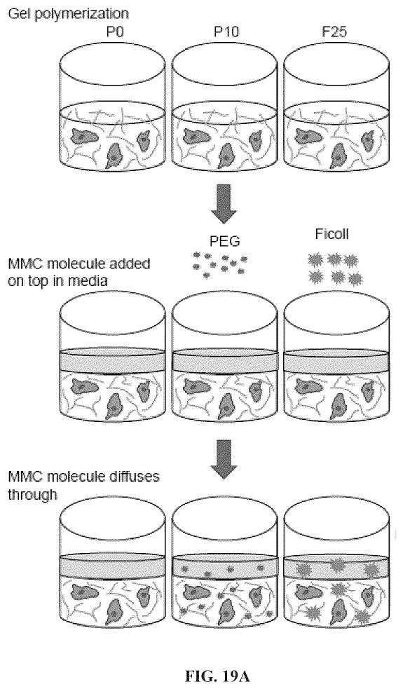

To explore the impact of fiber architecture on cancer cell behavior in a 3D collagen matrix, Applicant sought to develop a method for tuning fiber length in a collagen I hydrogel while simultaneously embedding cells. More specifically, the goal was to shorten fiber length of a 2.5 mg/ml collagen matrix without changing the density or stiffness of the matrix. The assembly of collagen I solution into a fibrous 3D matrix is driven by diffusion-limited growth of nucleated monomers (FIG. 18A). Crowding during collagen polymerization by the addition of 25 mg/ml of 400 Da Ficoll has previously been shown to tune fiber growth rate and architecture in cell-free conditions. However, studies suggest that Ficoll is cytotoxic. Thus, Applicant sought to test PEG as a MC agent for its biological inertness.

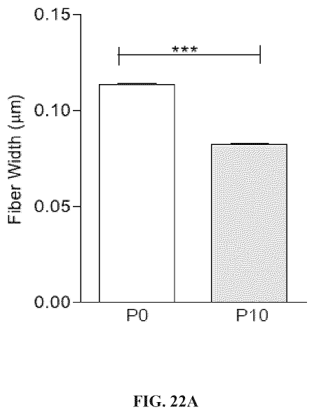

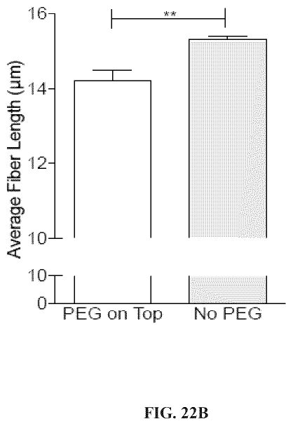

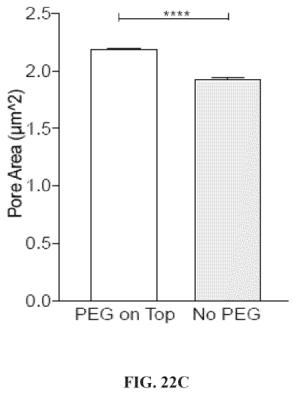

First Applicant tested the ability of PEG to alter collagen architecture. To do so, Applicant introduced increasing amounts of 8,000 Da PEG (0-10 mg/ml, labeled P0-P10) into a 2.5 mg/ml collagen I solution, then washed the gel after polymerization to remove the PEG, and finally imaged the resulting fiber architecture with reflection confocal microscopy. Increasing amounts of PEG led to gradual changes in collagen fibers (FIG. 18B). To ensure that the washing procedure effectively removed the PEG, Applicant conducted SEM imaging (FIG. 18C) of the P10 matrix with and without washing prior to fixation and sample processing for SEM. Applicant also imaged the PO matrix for comparison (FIG. 18C). These images show that PEG is not detectable in the collagen matrix after washing, confirming its function as an inert crowding agent. Quantitative analysis of reflection confocal images of the crowded matrices revealed a linear decrease in average fiber length, from 14.1 μm without PEG to 11.7 μm with 8 mg/ml of PEG mixed in during polymerization (FIG. 18D). Interestingly, this trend reversed between 8 and 10 mg/ml of PEG, where average fiber length increased slightly from 11.7 μm to 12.4 μm (FIG. 18D). The average pore size of the crowded matrices changed only slightly across all conditions, ranging from 1.75 to 2.2 μm² (FIG. 18E). Average fiber width, analyzed by SEM, varied marginally (~0.2 μm) with PEG crowding (FIG. 22A). These analyses indicated that although multiple matrix characteristics change simultaneously as a result of crowding, each feature follows its own characteristic dose-response relationship.

It is also interesting to quantify the relative heterogeneity of the fiber architecture, especially in the context of studying cell behavioral responses. Even the behavior of isogenic cells is heterogeneous, making it challenging to decouple intrinsic from extrinsic sources of cell heterogeneity. Using the coefficient of variation (CV) to assess heterogeneity in the fiber length and pore size of each condition, Applicant found that increased crowding results in a gradual decrease in CV (FIG. 18F-G). This indicates that in general, crowding homogenizes the matrix architecture. However, Applicant observed a slight increase in CV for the most crowded condition, 10 mg/ml PEG, as was observed for average pore size and fiber length.

To further characterize the biophysical properties of the crowded collagen constructs, Applicant measured their bulk and local elastic moduli using shear rheology and atomic force microscopy (AFM), respectively. Slightly differences in the bulk moduli were observed and statistically significant between the P4 crowded condition (14 Pa) and higher crowding conditions (~8 Pa, FIG. 18H). However, no significant differences in local moduli were observed when averaged over multiple locations and biological replicates (FIG. 18I). Thus, overall, matrix architecture was tuned without altering matrix stiffness and without changing the density of the collagen. This behavior may result from a balance between the increase in the connectivity of the network and a simultaneous weakening of the strength of the connections. It is important to note that the stiffness of 2.5 mg/ml collagen (here PO) has previously been shown to mimic normal breast tissue. All of the PEG crowded and non-crowded 2.5 mg/ml collagen constructs are within this range of stiffness and can be considered representative of the mechanical conditions cancer cells encounter during invasion and metastasis.

PEG Crowding Alone does not Directly Influence Cell Morphology or Migration Behavior

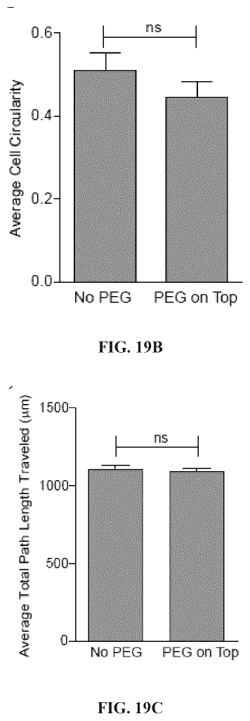

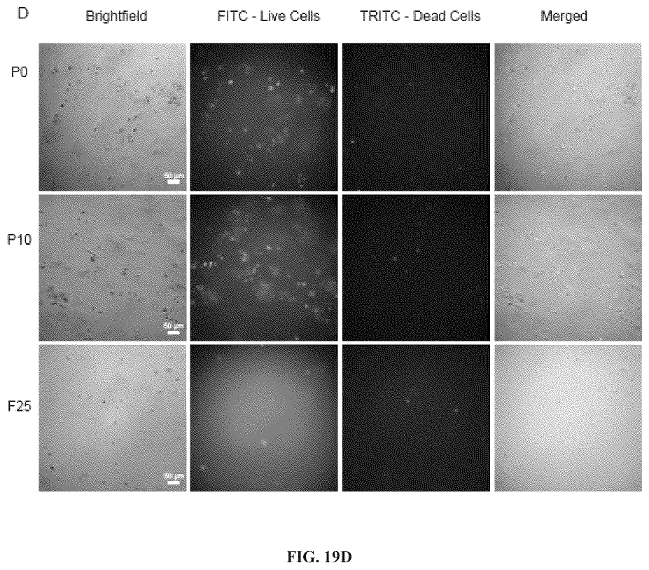

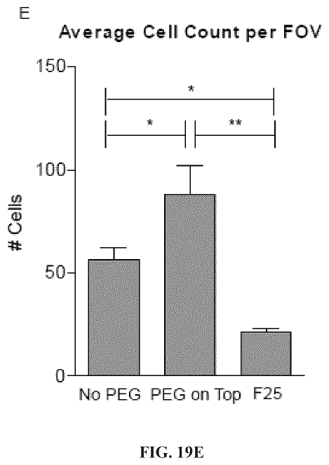

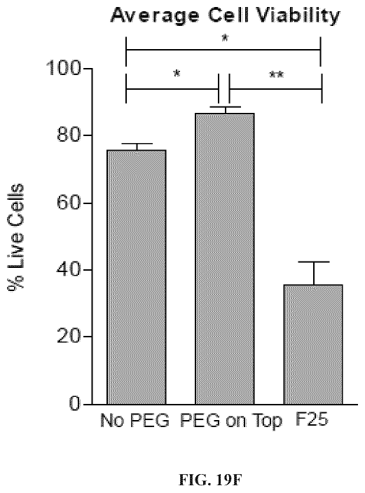

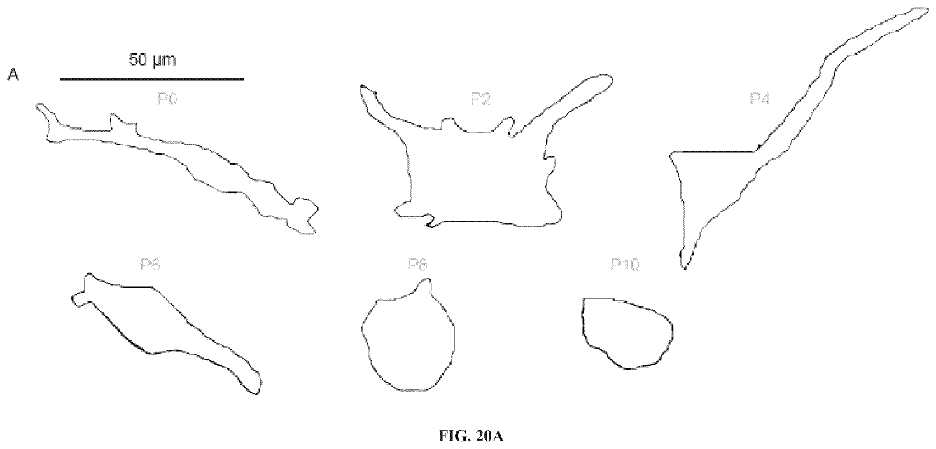

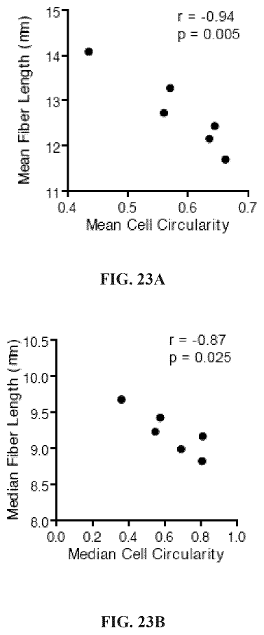

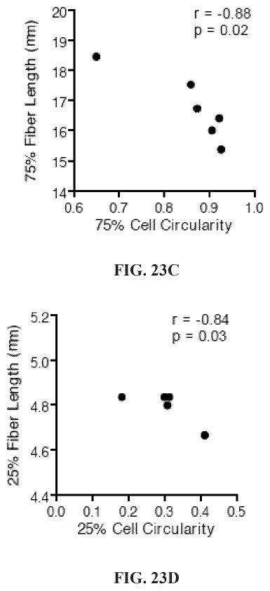

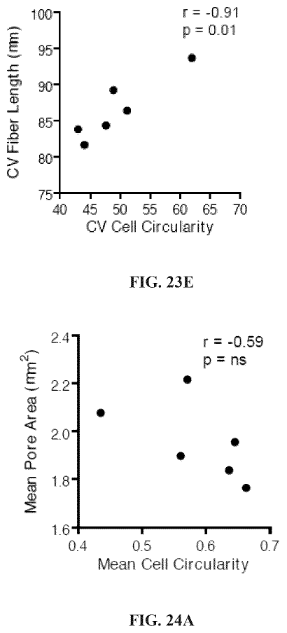

Having confirmed that PEG is an effective MC agent for tuning collagen architecture, Applicant next sought to determine whether it would directly influence cell behavior independently of its effects on matrix architecture. This control experiment was undertaken to ensure that even if the washing procedure did not remove all traces of PEG from the matrix, as suggested by the SEM images in FIG. 1C, cell behavioral differences result from fiber changes not cell-PEG interactions. To test this, Applicant embedded MDA-MB-231 breast cancer cells in a 2.5 mg/ml collagen matrix with no PEG added during polymerization. Then, Applicant added the maximum amount of PEG used for the matrix engineering experiments, 10 mg/ml, on top of the fully polymerized matrix and allowed the 8,000 Da molecules, radius of gyration ~3 nm, to freely diffuse into the interstitial spaces (FIG. 19A). In these experiments, no washing of the matrices was conducted. Reflection confocal analysis of the matrix architecture with and without PEG added on top revealed very slight differences in average fiber length (<1 μm) and pore size (<0.5 μm²) (FIGS. 23A-B). It is important to note that the large number of pores and fibers analyzed tends to generate statistical significance between conditions, even when differences are small. However, after 15 hours of culture in this control condition, no significant differences were observed in cell morphology or migration, as assessed by cell circularity and the total path length traveled by the cells over the first 15 hours respectively (FIGS. 19B-C).

Next, Applicant assessed the viability of the cells after one week of culture in the control and crowded conditions where the MC agent was not washed out. For comparison, Applicant also tested the effects of 25 mg/ml Ficoll 400 (400,000 Da) under the same condition, which has been used previously to tune collagen matrix architecture to approximately the same degree as Applicant accomplish here using 10 mg/ml PEG. Cells were seeded at the same initial density in all conditions. FIG. 19D shows micrographs of cells after one week. Total cell count was significantly lower in the Ficoll crowded conditions compared to the non-crowded and PEG crowded conditions after one week, indicating that Ficoll negatively impacted cell proliferation while PEG did not (FIG. 19D, left column, and FIG. 19E). Live-dead staining also revealed that cell viability was significantly reduced in Ficoll crowded conditions (FIG. 19F). Since Ficoll negatively impacted cell viability while PEG did not, and both achieved comparable changes to matrix architecture, Applicant conclude that PEG crowding is a more useful technique to alter the fiber architecture for embedded cell studies.

### 3D Collagen Fiber Topography Patterns Cell Shape

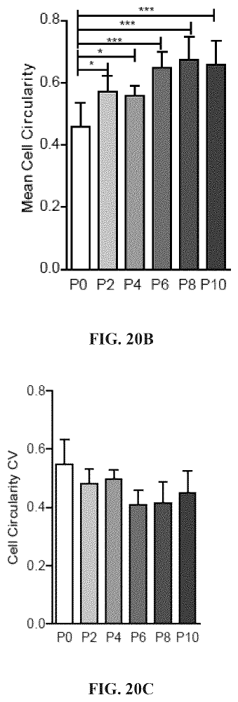

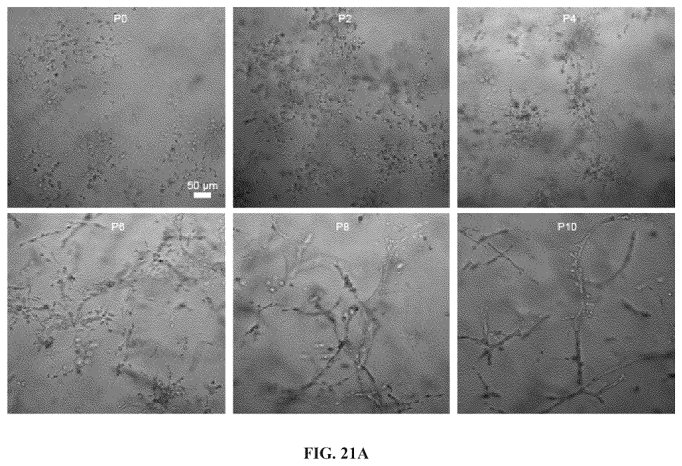

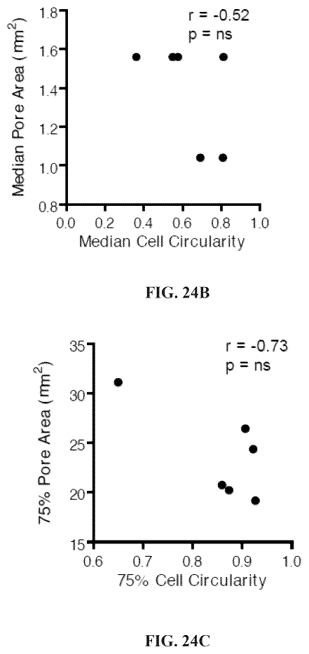

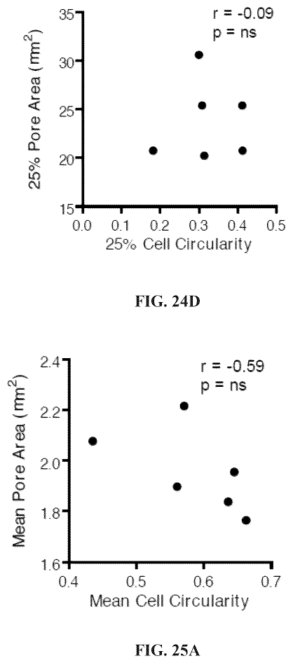

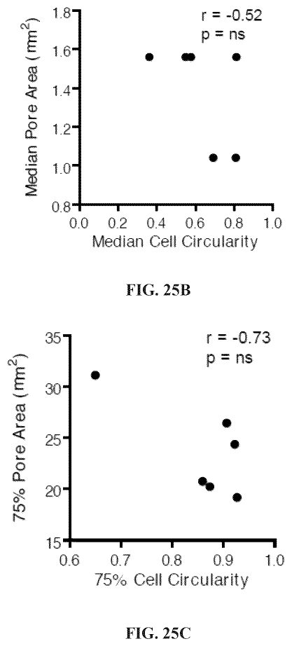

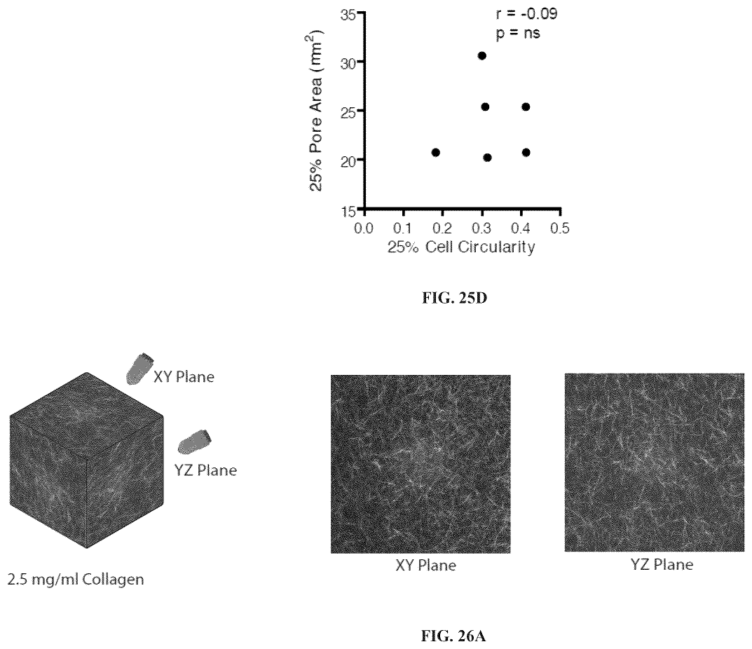

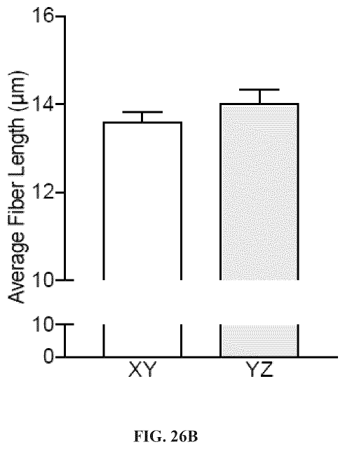

Having established PEG crowding as a method to modulate collagen fiber topology, Applicant next sought to quantify the influence of matrix architecture on the morphology and migration of embedded cancer cells. To do so, Applicant polymerized 2.5 mg/ml collagen with a low seeding density of MDA-MB-231 cells and 0-10 mg/ml PEG mixed in. After polymerization, the cell-laden gels were washed to remove the PEG, a process Applicant confirmed to be successful by SEM (FIG. 18C). Single cells were then monitored by timelapse microscopy. FIG. 20A highlights typical cell morphology differences observed in the crowded matrices as representative cell outlines in each condition after 15 hours. These trends in cell shape were stable throughout the first 15 hours following matrix polymerization and washing. Since cells were seeded sparsely and most had not yet divided during this time period, these morphology differences result from cell-matrix interactions as opposed to cell-cell interactions. Quantitative assessment of individual cell shapes at 15 hours revealed that cell circularity follows a similar trend as fiber length. As fibers were shortened by increased molecular crowding (FIG. 18D), cells became more rounded and less elongated (FIG. 20B) following a trend similar to that of fiber length. Mean, median, 75% values, and 25% values of fiber length each significantly predicted that of cell circularity (Pearson Correlation, Table 7 and FIGS. 23A-D). As another test of this relationship, Applicant would expect the CV of fiber length and the CV of cell circularity to follow a similar trend as well. That is, as fiber length becomes more homogenous with increased crowding, cell circularity would likewise become more homogenous. Indeed, as increased crowding causes lower CV of fiber length (FIG. 18F), cell circularity CV also decreased (FIG. 20C). The relationship between the CV of fiber length and CV of cell circularity was also significantly linear with a Pearson r=0.91 and p=0.01 (Table 7 FIG. 23E). Next Applicant compared the mean, median, 75% values, and 25% values of cell circularity to those of pore size and found that there was no significant relationship (FIGS. 24A-D). These findings suggest that cell shape is a linear function of 3D fiber length.

TABLE 8
Cell circularity in 3D collagen is a function of fiber length.

| | Mean | Median | 75% | 25% | CV |
|---|---|---|---|---|---|
| Cell Circularity | r = −0.94 | r = −0.87 | r = −0.88 | r = −0.84 | r = 0.91 |
| vs. | p = 0.005 | p = 0.025 | p = 0.021 | p = 0.035 | p = 0.01 |
| Fiber Length | | | | | |

3D Collagen Fiber Topography Modulates a Transition from Single Cell Migration Through Collective Cell Migration to Morphogenesis

To examine the impact of collagen fiber topography on breast cancer cell migration, Applicant monitored MDA-MB-231 cells for one week in each construct. A striking transition from single cell migration to collective migration was observed in the 2.5 mg/ml collagen matrices crowded with 6 mg/ml of PEG (P6) and higher. FIG. 21A shows representative micrographs of the breast cancer cells in each construct. Even more surprisingly, in P8 and P10 conditions the chain-like structures became more fused and smooth-edged, and other multicellular structures emerged at low frequency (FIGS. 21B and C). These structures resembled lobular and glandular structures of normal breast tissue. FIG. 4C shows the frequency of single cell, multicellular chain, and multicellular smooth structure phenotypes. It is important to reiterate that SEM imaging confirmed that the washing procedure effectively removed PEG after polymerization (FIG. 18C), and further, the presence of PEG added on top of the matrix in control experiments did not impact cell behavior (FIGS. 19B-C). Thus, without being bound by theory, it is concluded that matrix architecture drives these phenotypic transitions, from single cell migration through collective migration to morphogenesis.

Applicant next sought to identify which matrix feature(s) could be responsible for driving the switch from single to collective migration and further morphogenesis in the constructs, where the total density and overall stiffness of collagen was held constant. Since the phenotypic transition of breast cancer cells in the constructs was not gradual but sharp, Applicant hypothesized that matrix feature(s) could act in a thresholding capacity. To assess this, Applicant compared characteristic values for each matrix feature to the frequency at which Applicant observed single versus multicellular phenotypes across matrix conditions. Plotting the mean, median, and CV of fiber length against the frequency of single cell migration revealed that indeed a threshold in fiber length predicted the reduction in the single cell phenotype and the emergence of multicellular phenotypes (FIGS. 2 D-F). Likewise, an associated cell circularity threshold was identified (FIG. 21G), reinforcing the relationship between fiber length and cell shape. However, pore size could not reliably threshold the phenotypic switch (FIGS. 21H-K).

### Discussion

Applicant created a novel 3D collagen system that physically decouples both stiffness and density from fiber architecture to independently assess the impact of fiber architecture on cell behavior, the study reveals that when individual cells interact with different collagen matrix architectures, initial cell shape is a function of fiber length. Further, this interaction ultimately drives distinct modes of cell migration. Single cell migration is favored in matrices with long fibers whereas multicellular cell migration and morphogenesis is favored in matrices with short fibers. These two behaviors can be predicted based on a fiber length threshold and a related cell circularity threshold.

Previous work using hanging drops and 2D systems demonstrated that cell-ECM adhesion competes with cell-cell cohesion following physical principles related to surface tension. Without being bound by theory, it is possible that the short collagen fibers in the system restrict the size or stability of cell-ECM adhesion compared to longer fibers and thereby promote cell-cell cohesion. Alternatively, without being bound by theory, confinement in a rounded shape could alter the tensegrity of the cell, reducing the activation of cell surface integrins and their affinity for binding ECM. Tensile forces that are generated by contractile actomyosin filaments are resisted inside the cell by microtubules and outside the cell by the ECM and by adhesions to nearby cells. In rounded single cells, microtubules serve as the primary resistance to the pre-stressed cytoskeleton and also provide a mechanical force balance to a tensed network of chromosomes and nuclear scaffolds. This mechanical linkage could alter gene expression and cell behavior in a cell shape dependent way. Previous studies on 2D patterned substrates have shown that cellular geometry influences modular gene expression programs, differentiation, nuclear deformation, cytoskeleton reorganization, chromatin compaction, growth, apoptosis, and cell division. However, cell roundedness due to loss of attachment has also been shown to impair glucose uptake, inducing metabolic defects that drive gene expression changes.

A small percentage of the smooth multicellular structures Applicant observed in the P8 and P10 conditions (FIG. 21B) resemble normal lobular and acinar breast structures. Interestingly, Bissell and colleagues previously reported the reversion of a malignant breast cancer cell line into a normal acinar phenotype through the blockade of integrin beta 1 (ITGB1). Thus, a link may exist between the short fiber architecture, cell roundedness, and a reduction in the ability of ITGB1 to engage with the matrix. Further, the heterogeneity in the structures formed by the breast cancer cells in the system may represent different integrin-dependent responses as well as different metastatic capabilities. The more abundant network forming phenotype Applicant observed is reminiscent of the collective migration pattern implicated as the primary mode of tumor cell dissemination. Without being bound by theory, this collective behavior is thought to be linked to circulating tumor cells that are present as aggregates, which are predictive of poorer clinical outcomes. Thus, collagen architecture may influence the metastatic capabilities of cancer cells through modulation of migration phenotype. It is also be possible that the altered matrix enhances the sequestration of soluble factors and autocrine signaling or exposes cryptic collagen binding sites.

Increases in collagen matrix density induced “cellular jamming” in highly aggressive fibrosarcoma and melanoma cells leading to cell chain formation. These studies implicated pore size as the critical matrix feature inducing this migration switching phenomenon. The multicellular chain phenotype Applicant observed in the P6 construct is highly similar to that previously reported, where individual cell bodies are distinguishable but connected, resembling a pearl necklace. However, the fused networks, glands, and lobule structures formed in the P8 and P10 conditions are distinct. Further, Applicant found no relationship of pore size with the phenotypic switch. Without being bound by theory, these differences could arise from differences in cell type or from the fact that the system allowed us to hold collagen density constant while varying fiber architecture. Another distinction is that Applicant automated the measurement of pore sizes through image processing. However, the automated pore size measurement for the 2.5 mg/ml collagen condition (~2.2 μm²) is consistent with that reported previously by other groups using confocal reflection microscopy image analysis (~0.78-1.8 μm² pore areas for 2.5 mg/ml collagen; 2-5 μm² pore areas for 1.7 mg/ml) and cryo-EM (~3 μm² pore areas, 2 mg/ml collagen).

The successful use of varying concentrations of 8,000 Da PEG to modulate collagen matrix architecture begs the question of whether MC chain size and chemistry could be two additional matrix “tuning knobs” that could be further explored for collagen matrix engineering. The chemical nature of an MC agent as well as its size can determine its exclusion from molecular surfaces. Measurements of the exclusion of different molecular weights of PEG from proteins is largely consistent with the crowding by hard spheres model, where the radii is approximated by the radius of gyration of the PEG polymer. However, other measurements have shown that PEG polymers of different sizes are not always excluded from well defined cavities, as a hard-sphere model would require. A wide range of partial exclusion as a function of molecular size and concentration are possible. Without being bound by theory, this may explain, in part, the reverse in matrix parameter trends Applicant observed in the high concentration, 10 mg/ml PEG, condition. The nonlinearity of the behavior of collagen, a semiflexible crosslinked biopolymer network, in response to crowding by PEG, is not unexpected. A deep and predictive understanding of such networks has proven to be a daunting theoretical challenge in the field of soft matter and polymer physics. Another example of such non-linear behavior has been demonstrated by crowding actin filaments with PEG, where bundling is induced when the concentration of PEG exceeds a critical onset value. Indeed, the unique characteristics of biopolymers compared to synthetic polymers make their study highly important for fundamental biological understanding.

Also intriguing is the observation that cancer cell proliferation and viability increased slightly when PEG was added after collagen polymerization and maintained in culture for one week. Yet, no significant effect on cell morphology and migration was observed under these control conditions. Without being bound by theory, these findings suggest that molecular crowding may promote proliferation of tumor cells. Previous studies have found that PEG crowding can have the effect of increasing the hydration of proteins and imposing osmotic stress on cells. Both increased hydration and osmotic stress have been associated with cancerous tissues.

While collagen is only one of many matrix components within the tissue and tumor microenvironment, both clinical and in vivo studies have established the relevance of this particular ECM molecule. Collagen is both an independent clinical prognostic indicator of cancer progression and a driver of tumorigenesis and metastasis. As such, understanding how 3D collagen regulates cancer cell migration behavior is likely to provide useful insights into disease pathogenesis.

### CONCLUSIONS

A deeper understanding of the microenvironmental regulators of cancer cell migration could help identify therapies to combat metastasis and improve patient outcomes. By decoupling matrix architecture from matrix density and stiffness, the study identifies a novel role for collagen architecture in modulating cancer cell behavior. The techniques developed herein to modulate collagen architecture allowed us to identify relationships between fiber length, cell shape, and migration phenotype. Without being bound by theory, the same techniques could be extendable to investigations of metastatic migration in vivo, since the 3D matrix constructs are collagen and

PEG-based, non-toxic, and implantable. Without being bound by theory, these techniques may also be useful for stem cell and regenerative medicine studies as a means to control 3D cell shape and morphogenesis outcomes.

### Example 4 Methods

### Cancer Cell Culture

MDA-MB-231 breast cancer cells were ordered from ATCC (Manassas, VA) and cultured in Dulbecco's Modified Eagle's Medium (Life Technologies, Carlsbad, CA) supplemented with fetal bovine serum (Corning, Corning, NY) and gentamicin (Life Technologies, Carlsbad, CA), at 37 °C and 5% CO₂. Culture media was changed every other day as needed. Cells were cultured to confluence prior to being trypsinized and embedded inside of collagen gels. Cell laden gels were cultured for a week to observe long-term phenotypic differences.

### Collagen Gel Preparation

High concentration, rat tail acid extracted type I collagen was ordered form Corning (Corning, NY). MMC agents, PEG 8000 (8,000 Da) and Ficoll 400 (400,000 Da), were ordered in powder form from Sigma-Aldrich (St. Louis, MO) and reconstituted in PBS (Life Technologies, Carlsbad, CA) prior to usage. Trypsinized cells to be embedded, were first mixed with 1× reconstitution buffer composed of sodium bicarbonate, HEPES free acid, and nanopure water. Appropriate amounts of MMC agent, PEG or Ficoll, were then added to produce final concentrations of 0, 2, 4, 6, 8, and 10 mg ml⁻¹ PEG (denoted by PO, P2, P4, P6, P8, P10) or 25 mg ml⁻¹ Ficoll (denoted by F25). Afterwards, collagen solution was added to the mixture for a final concentration of 2.5 mg ml⁻¹ collagen. Finally, pH of the final mixture was adjusted using IN sodium hydroxide, prior to polymerization via incubation at 37 °C (~20-30 minutes). Gels were prepared inside of 48-well plates with a total volume of 200 μl. Following gel polymerization and solidification, MMC molecules were washed out of the collagen gels by rinsing with PBS 3× for 5 min each. Cell culture media was then added on top of the gels after and changed every two days as necessary.

### MMC Control Experiments

To ensure that the MMC molecules being used were truly inert, Applicant investigated any potential effect that the MMC molecules may have on cells independent of the changing matrix architecture. Applicant conducted a series of MMC control experiments, where MDA-MB-231 cells were embedded inside of three 2.5 mg ml⁻¹ collagen gels. After the gels had polymerized, normal culture media was added on top to one of the gel, while the other two either had 10 mg ml⁻¹ PEG or 25 mg ml⁻¹ Ficoll added into the media on top. The MMC molecules were left in the media and allowed to diffuse down into the gel with the cells. Subsequent media changes would also include appropriate amounts of MMC agent to maintain the PEG and Ficoll concentrations in the gel.

### Confocal Reflection Imaging of Collagen Architecture

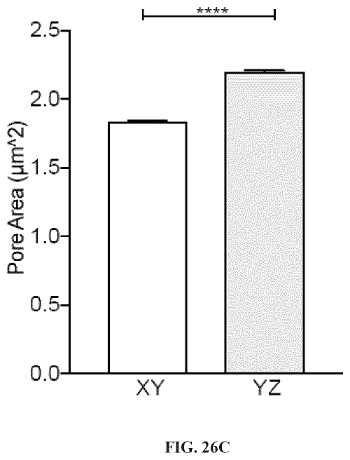

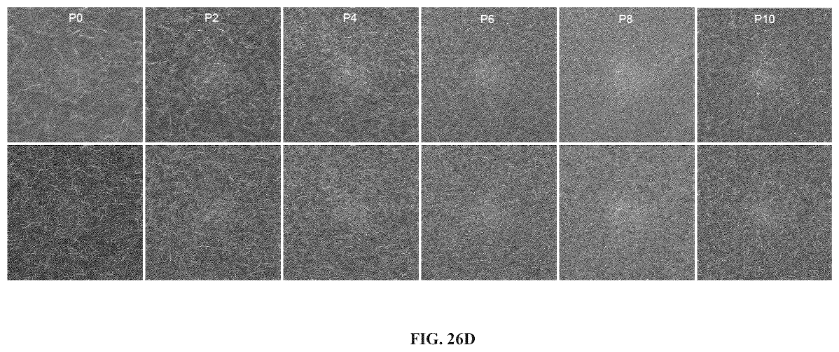

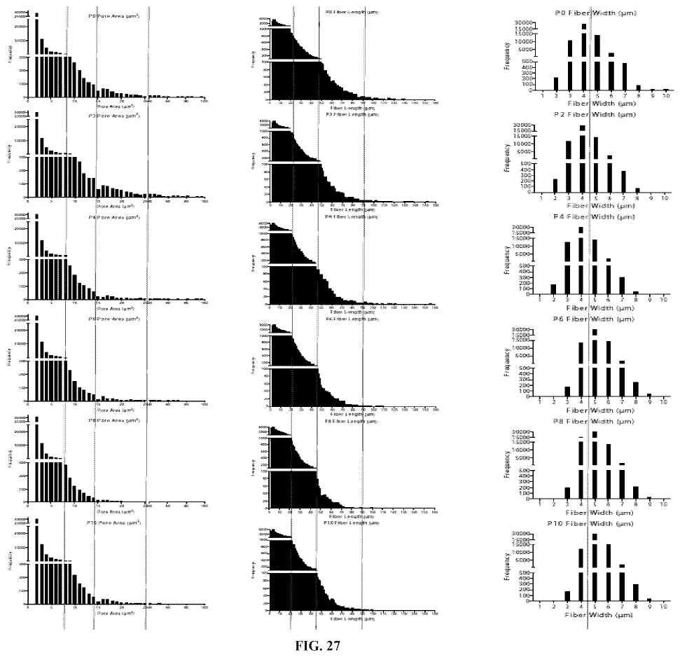

Collagen matrix architecture and topography was investigated by imaging gels using confocal reflection microscopy (CRM) using a Leica SP5 inverted confocal microscope, equipped with a 20× immersion objective (NA=1.0). Collagen fibers were imaged by exciting with and collecting backscattered light at 488 nm. Confocal reflection imaging is restricted to fibers that are oriented within 50° of the imaging plane. To verify that the gels are isotropic in their fiber structure, Applicant imaged a gel from the top and from one of the sides (XY and YZ planes), using the same imaging settings. FIGS. 26A-C shows that differences were negligible.

### Time Lapse Imaging Microscopy

Time lapse microscopy was conducted using a Nikon Ti-E inverted microscope, equipped with a stage top incubation system, to analyze cell motility and migration behavior, morphology, and proliferation and viability. Cells were allowed to settle in the collagen gel in the incubator for approximately 7 h after gel polymerization; time-lapse imaging began at around the 8th hour after the cells were embedded into the collagen gels. Each gel was imaged over 6 fields of view (FOV) for a period of 15 h, with images being taken every 2 min.

### Cell Proliferation Assay

Cell viability was assessed using a Live and Dead Cell Assay (Abcam, Cambridge, UK). Intact, viable cells fluoresce green (imaged under FITC channel) while dead cells fluoresce red (imaged under TRITC channel). The average number of live cells, dead cells, and live cell viability percentages were calculated over 4 FOVs per condition. Live cell viability % is defined as the number of live cells/the total number of cells * 100.

### Matrix Analysis

All matrix analyses were done using the CRM images of the collagen gels in each condition over 3 FOVs. Fiber analysis was conducted in CT-FIRE v1.3 by measuring individual fiber length and width as previously published. Minimum fiber length, dangler length threshold (thresh_dang_L), short fiber length threshold (thresh_short_L), distance for linking same-oriented fibers (thresh_linkd), and minimum length of a free fiber (thresh_flen), were all set to three pixels. Default settings were used for all other fiber extraction parameters and output figure controls. Settings were optimized to detect and analyze discrete fibers in CRM images on scales of 0.72 μm per pixel. Examples of the fibers found by CT-FIRE are shown in FIG. 26D, along with the associated reflection confocal micrographs.

Pore size was calculated using NIS-Elements software (Nikon) as the 2D area encompassed by fibers. The total pore area (€) as a 2D approximation of the 3D tissue ultrastructure (¿) can be described as:

$${ε}^{′} = ε exp ( \frac{- α {D}_{f}}{4 ε} ) \qquad Eq 1$$

as long as the stereological assumption is met. This assumption was validated by imaging XY and YZ planes of a 2.5 mg/ml collagen gel and analyzing the fiber length (no significant difference), pore area (<0.5 μm² difference), which are shown in FIGS. 26A-C. Additionally, if the imaging depth of field (D_{f}) is small enough ε′=ε. Applicant calculated the D_{f} from:

$${D}_{f} = \frac{λ n}{{NA}^{2}} + \frac{n}{M \cdot NA} e \qquad Eq 2$$

to be ~0.67 microns. This is smaller than the pixel size in the system and smaller than the expected ε, which makes the fraction in Eq 1<1 and suggests that ε′=ε is a valid approximation. These analyses suggest that the 2D confocal micrographs are a close representation of the 3D architecture. Pre-processing of images were conducted by implementing a Gauss-Laplace Sharpen set to a power of 2 and then by using a Rolling Ball Correction with a rolling ball radius of 15. The contrast of all images were equalized, then images were binarized by thresholding to the same range. Next the automated measurement tool was used to measure pore areas in the binarized images. Single pixel pore values were attributed to speckle noise and removed from all conditions. Homo-/heterogeneity of the matrices was characterized by calculating the coefficient of variation (CV) of fiber length and pore size data distributions.

### Rheometry

Rheological measurements were conducted as shown previously with a TA Instruments AR-G2 Rheometer. A parallel-plate geometry (20 mm diameter) with a 1000 μm gap height was used on 500 μl collagen gels. For each condition, a strain sweep at frequency of 1 rad/s was recorded to determine each respective linear viscoelastic region. The storage modulus (G′) and loss modulus (G″) were recorded over frequencies of 0.25-100 rad/s for each condition. The storage modulus at 1 rad/s was reported for each condition.

### Atomic Force Microscopy (AFM)

AFM was performed to measure local collagen gel stiffness as previously described59. Briefly, nano-indentations were performed using a MFP-3D Bio Atomic Force Microscope (Oxford Instruments) mounted on a Ti-U fluorescent inverted microscope (Nikon Instruments). A pyrex-nitride probe (spring constants ~0.04 N/m, NanoAndMore USA Corporation, cat #PNP-TR) with a pyramid tip was calibrated using a thermal noise method provided by the Igor 6.34A software (WaveMetrics). Samples were loaded on the AFM, submersed in phosphate buffered saline (PBS), and indented at a velocity of 2 μm/s. Tip deflections were converted to indentation force for all samples using their respective tip spring constants and Hooke's Law. Elastic modulus was calculated based on a Hertz-based fit using a built-in code written in the Igor 6.34A software.

### Scanning Electron Microscope (SEM)

Collagen gels were prepared at 2.5 mg/mL concentration with and without the addition of 10 mg/mL 8 KDa PEG, then placed in a humidified incubator (37 °C) until fully polymerized as described above. The samples polymerized in the presence of PEG were separated into washed and not washed preparations. To wash the PEG after polymerization, PBS was added on top of the gel and placed in the incubator for 5 minutes 3 times. Next, all samples were fixed with 4% PFA for 1 hour at room temperature and the washed 3× with PBS. The samples were then dehydrated by treating them with increasing concentrations of ethanol (50% to 100%). Samples immersed in 100% ethanol were subjected to critical point drying (Autosamdri-815, Tousimis, Rockville, MD, USA), coated with a thin layer of Iridium (Emitech K575X, Quorum technologies, Ashford, UK) and imaged using a Zeiss sigma 500 SEM.

### Cell Analysis

Individual cells in the time lapse videos were tracked in Metamorph for motility characterization. Within the 15 h time lapse window, cells were analyzed in terms of the total path length traveled, their average speed, the invasion distance (displacement), and the persistence of their migration (defined as the invasion distance/the total path length traveled). Cell morphology analysis was conducted using images of the cells during the 15^{th} hour after seeding in the gel, in terms of circularity (computed as: [4*π*area]/perimeter²).

### Correlation Analysis

Correlations were calculated in terms of the Pearson correlation coefficient. Correlations were drawn between various matrix parameters to evaluate whether they had been decoupled, as well as between matrix and cell parameters to investigate cell-matrix interactions.

### Statistics

Data presented in bar graph format was analyzed using one-way analysis of variance (ANOVA) followed by Tukey or Newman-Keuls post hoc test in GraphPad Prism (v5). Correlation plots were analyzed by Pearson Correlation in GraphPad Prism (v5). Pearson r correlation coefficient and two-tailed p values are reported. N=3 biological replicates for each condition tested. Statistical significance was set at values of p<0.05 and reported as p<0.001, ***; p<0.01, **; p<0.05, *.

### REFERENCES

The following articles are referenced in the disclosure hereinabove and are incorporated by reference in their entirety:
Palmer, T. D., Ashby, W. J., Lewis, J. D. & Zijlstra, A. Targeting tumor cell motility to prevent metastasis. Adv Drug Deliv Rev 63, 568-581, doi:10.1016/j.addr.2011.04.008 (2011).
Fang, M., Yuan, J., Peng, C. & Li, Y. Collagen as a double-edged sword in tumor progression. Tumour Biol 35, 2871-2882, doi:10.1007/s13277-013-1511-7 (2014).
Provenzano, P. P. et al. Collagen density promotes mammary tumor initiation and progression. Biomed Central Medicine 6, 11, doi:10.1186/1741-7015-6-11 (2008).
Zhu, G. G. et al. Immunohistochemical study of type I collagen and type I pN-collagen in benign and malignant ovarian neoplasms. Cancer 75, 1010-1017 (1995).
Drifka, C. R. et al. Periductal stromal collagen topology of pancreatic ductal adenocarcinoma differs from that of normal and chronic pancreatitis. Mod Pathol 28, 1470-1480, doi:10.1038/modpathol.2015.97 (2015).
Huijbers, I. J. et al. A role for fibrillar collagen deposition and the collagen internalization receptor endo180 in glioma invasion. PLoS One 5, e9808, doi:10.1371/journal.pone.0009808 (2010).
Conklin, M. W. et al. in Am J Pathol Vol. 178 1221-1232 (2011).
Gligorijevic, B., Bergman, A. & Condeelis, J. Multiparametric classification links tumor microenvironments with tumor cell phenotype. PLoS Biol 12, e1001995, doi:10.1371/journal.pbio.1001995 (2014).
Giampieri, S. et al. Localized and reversible TGFbeta signalling switches breast cancer cells from cohesive to single cell motility. Nat Cell Biol 11, 1287-1296, doi:10.1038/ncb 1973 (2009).
Fraley, S. I. et al. Three-dimensional matrix fiber alignment modulates cell migration and MT1-MMP utility by spatially and temporally directing protrusions. Sci Rep 5, 14580, doi:10.1038/srep14580 (2015).
Kumar, S. & Weaver, V. M. Mechanics, malignancy, and metastasis: The force journey of a tumor cell. Cancer Metastasis Rev 28, 113-127, doi:10.1007/s10555-008-9173-4 (2009).
Folberg, R., Hendrix, M. J. & Maniotis, A. J. Vasculogenic mimicry and tumor angiogenesis. Am J Pathol 156, 361-381, doi:10.1016/s0002-9440 (10) 64739-6 (2000).
Maniotis, A. J. et al. Vascular channel formation by human melanoma cells in vivo and in vitro: vasculogenic mimicry. Am J Pathol 155, 739-752, doi:10.1016/s0002-9440 (10) 65173-5 (1999).
Hendrix, M. J. et al. Expression and functional significance of VE-cadherin in aggressive human melanoma cells: role in vasculogenic mimicry. Proc Natl Acad Sci USA 98, 8018-8023, doi:10.1073/pnas. 131209798 (2001).
Zhang, J. G. et al. ROCK is involved in vasculogenic mimicry formation in hepatocellular carcinoma cell line. PLoS One 9, e107661, doi:10.1371/journal.pone.0107661 (2014).
Williamson, S. C. et al. Vasculogenic mimicry in small cell lung cancer. Nat Commun 7, 13322, doi:10.1038/ncomms13322 (2016).
Liu, T. J. et al. CD133+ cells with cancer stem cell characteristics associates with vasculogenic mimicry in triple-negative breast cancer. Oncogene 32, 544-553, doi:10.1038/one.2012.85 (2013).
Wagenblast, E. et al. A model of breast cancer heterogeneity reveals vascular mimicry as a driver of metastasis. Nature 520, 358-362, doi:10.1038/nature14403 (2015).
Misra, R. M., Bajaj, M. S. & Kale, V. P. Vasculogenic mimicry of HT1080 tumour cells in vivo: critical role of HIF-1alpha-neuropilin-1 axis. PLoS One 7, e50153, doi:10.1371/journal.pone.0050153 (2012).
Fraley, S. I. et al. A distinctive role for focal adhesion proteins in three-dimensional cell motility. Nature Cell Biology 12, 598-604 (2010).
Fraley, S. I., Feng, Y., Giri, A., Longmore, G. D. & Wirtz, D. Dimensional and temporal controls of three-dimensional cell migration by zyxin and binding partners. Nature Communications 3, 719, doi:10.1038/ncomms1711 (2012).
Fraley, S. I., Feng, Y., Wirtz, D. & Longmore, G. D. Reply: reducing background fluorescence reveals adhesions in 3D matrices. Nature Cell Biology 13, 5-7, doi: doi:10.1038/ncb0111-5 (2010).
Saliba, A. E., Westermann, A. J., Gorski, S. A. & Vogel, J. Single-cell RNA-seq: advances and future challenges. Nucleic Acids Res 42, 8845-8860, doi:10.1093/nar/gku555 (2014).
Demou, Z. N. & Hendrix, M. J. Microgenomics profile the endogenous angiogenic phenotype in subpopulations of aggressive melanoma. J Cell Biochem 105, 562-573, doi:10.1002/jcb.21855 (2008).
Hendrix, M. J., Seftor, E. A., Hess, A. R. & Seftor, R. E. Vasculogenic mimicry and tumour-cell plasticity: lessons from melanoma. Nat Rev Cancer 3, 411-421, doi:10.1038/nrc1092 (2003).
Valiente, M. et al. Serpins promote cancer cell survival and vascular co-option in brain metastasis. Cell 156, 1002-1016, doi:10.1016/j.cell.2014.01.040 (2014).
Barczyk, M., Carracedo, S. & Gullberg, D. Integrins. Cell Tissue Res 339, 269-280, doi:10.1007/s00441-009-0834-6 (2010).
Brinkerhoff, C. J. & Linderman, J. J. Integrin Dimerization and Ligand Organization: Key Components in Integrin Clustering for Cell Adhesion. Tissue Eng Part A 11, 865-876, doi:10.1089/ten.2005.11.865 (2005).
Taipale, J. & Keski-Oja, J. Growth factors in the extracellular matrix. Faseb j 11, 51-59 (1997).
Przybyla, L. M., Theunissen, T. W., Jaenisch, R. & Voldman, J. Matrix remodeling maintains embryonic stem cell self-renewal by activating Stat3. Stem Cells 31, 1097-1106, doi:10.1002/stem. 1360 (2013).
Koledova, Z. et al. SPRY1 regulates mammary epithelial morphogenesis by modulating EGFR-dependent stromal paracrine signaling and ECM remodeling. Proc Natl Acad Sci USA 113, E5731-5740, doi:10.1073/pnas. 1611532113 (2016).
Paszek, M. J. et al. Tensional homeostasis and the malignant phenotype. Cancer Cell 8, 241-254, doi:10.1016/j.ccr.2005.08.010 (2005).
Wei, S. C. et al. Matrix stiffness drives epithelial-mesenchymal transition and tumour metastasis through a TWIST1-G3BP2 mechanotransduction pathway. Nat Cell Biol 17, 678-688, doi:10.1038/ncb3157 (2015).
Ramanujan, S. et al. Diffusion and convection in collagen gels: implications for transport in the tumor interstitium. Biophys J 83, 1650-1660, doi:10.1016/s0006-3495 (02) 73933-7 (2002).
Abaci, H. E., Truitt, R., Tan, S. & Gerecht, S. Unforeseen decreases in dissolved oxygen levels affect tube formation kinetics in collagen gels. Am J Physiol Cell Physiol 301, C431-440, doi:10.1152/ajpcell.00074.2011 (2011).
van der Schaft, D. W. et al. Tumor cell plasticity in Ewing sarcoma, an alternative circulatory system stimulated by hypoxia. Cancer Res 65, 11520-11528, doi:10.1158/0008-5472.can-05-2468 (2005).
Sun, B. et al. Hypoxia influences vasculogenic mimicry channel formation and tumor invasion-related protein expression in melanoma. Cancer Lett 249, 188-197, doi:10.1016/j.canlet.2006.08.016 (2007).
Thienpont, B. et al. Tumour hypoxia causes DNA hypermethylation by reducing TET activity. Nature 537, 63-68, doi:10.1038/nature19081 (2016).
Cimmino, F. et al. Inhibition of hypoxia inducible factors combined with all-trans retinoic acid treatment enhances glial transdifferentiation of neuroblastoma cells. Sci Rep 5, 11158, doi:10.1038/srep11158 (2015).
Janaszak-Jasiecka, A. et al. miR-429 regulates the transition between Hypoxia-Inducible Factor (HIF) 1A and HIF3A expression in human endothelial cells. Sci Rep 6, 22775, doi:10.1038/srep22775 (2016).
Seftor, R. E. et al. Tumor cell vasculogenic mimicry: from controversy to therapeutic promise. Am J Pathol 181, 1115-1125, doi:10.1016/j.ajpath.2012.07.013 (2012).
Lahlou, H. & Muller, W. J. in Breast Cancer Res Vol. 13 229 (2011).
Seftor, R. E. et al. Cooperative interactions of laminin 5 gamma2 chain, matrix metalloproteinase-2, and membrane type-1-matrix/metalloproteinase are required for mimicry of embryonic vasculogenesis by aggressive melanoma. Cancer Res 61, 6322-6327 (2001).
Castro-Sanchez, L., Soto-Guzman, A., Guaderrama-Diaz, M., Cortes-Reynosa, P. & Salazar, E. P. Role of DDR1 in the gelatinases secretion induced by native type IV collagen in MDA-MB-231 breast cancer cells. Clin Exp Metastasis 28, 463-477, doi:10.1007/s10585-011-9385-9 (2011).
Howlader, N. et al. US incidence of breast cancer subtypes defined by joint hormone receptor and HER2 status. J Natl Cancer Inst 106, doi:10.1093/jnci/dju055 (2014).
Uhlen, M. et al. Proteomics. Tissue-based map of the human proteome. Science 347, 1260419, doi:10.1126/science.1260419 (2015).
Jain, N., Iyer, K. V., Kumar, A. & Shivashankar, G. V. Cell geometric constraints induce modular gene-expression patterns via redistribution of HDAC3 regulated by actomyosin contractility. Proc Natl Acad Sci USA 110, 11349-11354, doi:10.1073/pnas. 1300801110 (2013).
Jean, R. P., Gray, D. S., Spector, A. A. & Chen, C. S. Characterization of the nuclear deformation caused by changes in endothelial cell shape. J Biomech Eng 126, 552-558 (2004).
Versaevel, M., Grevesse, T. & Gabriele, S. Spatial coordination between cell and nuclear shape within micropatterned endothelial cells. Nat Commun 3, 671, doi:10.1038/ncomms1668 (2012).
Thery, M., Pepin, A., Dressaire, E., Chen, Y. & Bornens, M. Cell distribution of stress fibres in response to the geometry of the adhesive environment. Cell Motil Cytoskeleton 63, 341-355, doi:10.1002/cm.20126 (2006).
Vergani, L., Grattarola, M. & Nicolini, C. Modifications of chromatin structure and gene expression following induced alterations of cellular shape. Int J Biochem Cell Biol 36, 1447-1461, doi:10.1016/j.biocel.2003.11.015 (2004).
Yang, J. P. et al. Tumor vasculogenic mimicry predicts poor prognosis in cancer patients: a meta-analysis. Angiogenesis 19, 191-200, doi:10.1007/s10456-016-9500-2 (2016).
Wu, P. H., Giri, A., Sun, S. X. & Wirtz, D. Three-dimensional cell migration does not follow a random walk. Proc Natl Acad Sci USA 111, 3949-3954, doi:10.1073/pnas. 1318967111 (2014).
Wu, P. H., Giri, A. & Wirtz, D. Statistical analysis of cell migration in 3D using the anisotropic persistent random walk model. Nat Protoc 10, 517-527, doi:10.1038/nprot.2015.030 (2015).
Langmead, B. & Salzberg, S. L. Fast gapped-read alignment with Bowtie 2. Nat Methods 9, 357-359, doi:10.1038/nmeth. 1923 (2012).
Roberts, A. & Pachter, L. Streaming fragment assignment for real-time analysis of sequencing experiments. Nat Methods 10, 71-73, doi:10.1038/nmeth.2251 (2013).
Maere, S., Heymans, K. & Kuiper, M. BiNGO: a Cytoscape plugin to assess overrepresentation of gene ontology categories in biological networks. Bioinformatics 21, 3448-3449, doi:10.1093/bioinformatics/bti551 (2005).
Shalem, O. et al. Genome-scale CRISPR-Cas9 knockout screening in human cells. Science 343, 84-87, doi:10.1126/science. 1247005 (2014).
Wilks, C. et al. The Cancer Genomics Hub (CGHub): overcoming cancer through the power of torrential data. Database (Oxford) 2014, doi:10.1093/database/bau093 (2014).
Patro, R., Mount, S. M. & Kingsford, C. Sailfish enables alignment-free isoform quantification from RNA-seq reads using lightweight algorithms. Nat Biotechnol 32, 462-464, doi:10.1038/nbt.2862 (2014).
Gross, A. M. et al. Multi-tiered genomic analysis of head and neck cancer ties TP53 mutation to 3p loss. Nat Genet 46, 939-943, doi:10.1038/ng.3051 (2014).
Ceballos, D. et al. Magnetically aligned collagen gel filling a collagen nerve guide improves peripheral nerve regeneration. Experimental neurology 158, 290-300, doi:10.1006/exnr. 1999.7111 (1999).
Dubey, N., Letourneau, P. C. & Tranquillo, R. T. Neuronal contact guidance in magnetically aligned fibrin gels: effect of variation in gel mechano-structural properties. Biomaterials 22, 1065-1075 (2001).
Oliveira, A. L. et al. Aligned silk-based 3-D architectures for contact guidance in tissue engineering. Acta biomaterialia 8, 1530-1542, doi:10.1016/j.actbio.2011.12.015 (2012).
Haeger, A., Krause, M., Wolf, K. & Friedl, P. Cell jamming: collective invasion of mesenchymal tumor cells imposed by tissue confinement. Biochimica et biophysica acta 1840, 2386-2395, doi:10.1016/j.bbagen.2014.03.020 (2014).
Jiang, G., Huang, A. H., Cai, Y., Tanase, M. & Sheetz, M. P. Rigidity sensing at the leading edge through alphavbeta3 integrins and RPTPalpha. Biophysical journal 90, 1804-1809, doi:10.1529/biophysj. 105.072462 (2006).
Lo, C. M., Wang, H. B., Dembo, M. & Wang, Y. L. Cell movement is guided by the rigidity of the substrate. Biophysical journal 79, 144-152, doi:10.1016/s0006-3495 (00) 76279-5 (2000).
Paszek, M. J. et al. Tensional homeostasis and the malignant phenotype. Cancer cell 8, 241-254, doi:10.1016/j.ccr.2005.08.010 (2005).
Fraley, S. I., Feng, Y., Giri, A., Longmore, G. D. & Wirtz, D. Dimensional and temporal controls of three-dimensional cell migration by zyxin and binding partners. Nature communications 3, 719, doi:10.1038/ncomms1711 (2012).
Fraley, S. I. et al. A distinctive role for focal adhesion proteins in three-dimensional cell motility. Nature cell biology 12, 598-604, doi:10.1038/ncb2062 (2010).
Yue, X., Lukowski, J. K., Weaver, E. M., Skube, S. B. & Hummon, A. B. Quantitative Proteomic and Phosphoproteomic Comparison of 2D and 3D Colon Cancer Cell Culture Models. Journal of proteome research 15, 4265-4276, doi:10.1021/acs.jproteome.6b00342 (2016).
Ray, A., Slama, Z. M., Morford, R. K., Madden, S. A. & Provenzano, P. P. Enhanced Directional Migration of Cancer Stem Cells in 3D Aligned Collagen Matrices. Biophysical journal 112, 1023-1036, doi:10.1016/j.bpj.2017.01.007 (2017).
Zeng, Y. N., Kang, Y. L., Rau, L. R., Hsu, F. Y. & Tsai, S. W. Construction of cell-containing, anisotropic, three-dimensional collagen fibril scaffolds using external vibration and their influence on smooth muscle cell phenotype modulation. Biomedical materials (Bristol, England), doi:10.1088/1748-605X/aa766d (2017).
Antman-Passig, M., Levy, S., Gartenberg, C., Schori, H. & Shefi, O. Mechanically Oriented 3D Collagen Hydrogel for Directing Neurite Growth. Tissue engineering. Part A 23, 403-414, doi:10.1089/ten.TEA.2016.0185 (2017).
Provenzano, P. P., Eliceiri, K. W., Inman, D. R. & Keely, P. J. Engineering three-dimensional collagen matrices to provide contact guidance during 3D cell migration. Current protocols in cell biology Chapter 10, Unit 10.17, doi:10.1002/0471143030.cb1017s47 (2010).
Engler, A. J. et al. Myotubes differentiate optimally on substrates with tissue-like stiffness: pathological implications for soft or stiff microenvironments. The Journal of cell biology 166, 877-887, doi:10.1083/jcb.200405004 (2004).
Chen, C., Loe, F., Blocki, A., Peng, Y. & Raghunath, M. Applying macromolecular crowding to enhance extracellular matrix deposition and its remodeling in vitro for tissue engineering and cell-based therapies. Advanced drug delivery reviews 63, 277-290, doi:10.1016/j.addr.2011.03.003 (2011).
Dewavrin, J. Y., Hamzavi, N., Shim, V. P. & Raghunath, M. Tuning the architecture of three-dimensional collagen hydrogels by physiological macromolecular crowding. Acta biomaterialia 10, 4351-4359, doi:10.1016/j.actbio.2014.06.006 (2014).
Parkinson, J., Kadler, K. E. & Brass, A. Simple physical model of collagen fibrillogenesis based on diffusion limited aggregation. Journal of molecular biology 247, 823-831, doi:10.1006/jmbi. 1994.0182 (1995).
Salvalaggio, P. R. et al. Islet filtration: a simple and rapid new purification procedure that avoids ficoll and improves islet mass and function. Transplantation 74, 877-879, doi:10.1097/01.tp.0000028781.41729.5b (2002).
Szymanska, P., Gritti, N., Keegstra, J. M., Soltani, M. & Munsky, B. Using noise to control heterogeneity of isogenic populations in homogenous environments. Physical biology 12, 045003, doi:10.1088/1478-3975/12/4/045003 (2015).
Abdallah, B. Y. et al. in Cell Cycle Vol. 12 3640-3649 (2013).
Depalle, B., Qin, Z., Shefelbine, S. J. & Buehler, M. J. Influence of cross-link structure, density and mechanical properties in the mesoscale deformation mechanisms of collagen fibrils. J Mech Behav Biomed Mater 52, 1-13, doi:10.1016/j.jmbbm.2014.07.008 (2015).
Rubinson, K. A. & Krueger, S. Poly(ethylene glycol) s 2000-8000 in water may be planar: A small-angle neutron scattering (SANS) structure study. Polymer 50, 4852-4858 (2009).
Sokol, E. S. et al. Growth of human breast tissues from patient cells in 3D hydrogel scaffolds. Breast cancer research: BCR 18, 19, doi:10.1186/s13058-016-0677-5 (2016).
Steinberg, M. S. Differential adhesion in morphogenesis: a modern view. Curr Opin Genet Dev 17, 281-286, doi:10.1016/j.gde.2007.05.002 (2007).
Coughlin, M. F. & Stamenovic, D. A tensegrity model of the cytoskeleton in spread and round cells. J Biomech Eng 120, 770-777 (1998).
Ingber, D. E., Wang, N. & Stamenovic, D. Tensegrity, cellular biophysics, and the mechanics of living systems. Rep Prog Phys 77, 046603, doi:10.1088/0034-4885/77/4/046603 (2014).
Jain, N., Iyer, K. V., Kumar, A. & Shivashankar, G. V. Cell geometric constraints induce modular gene-expression patterns via redistribution of HDAC3 regulated by actomyosin contractility. Proceedings of the National Academy of Sciences of the United States of America 110, 11349-11354, doi:10.1073/pnas. 1300801110 (2013).
Jean, R. P., Gray, D. S., Spector, A. A. & Chen, C. S. Characterization of the nuclear deformation caused by changes in endothelial cell shape. Journal of biomechanical engineering 126, 552-558 (2004).
Versaevel, M., Grevesse, T. & Gabriele, S. Spatial coordination between cell and nuclear shape within micropatterned endothelial cells. Nature communications 3, 671, doi:10.1038/ncomms1668 (2012).
Thery, M., Pepin, A., Dressaire, E., Chen, Y. & Bornens, M. Cell distribution of stress fibres in response to the geometry of the adhesive environment. Cell motility and the cytoskeleton 63, 341-355, doi:10.1002/cm.20126 (2006).
Vergani, L., Grattarola, M. & Nicolini, C. Modifications of chromatin structure and gene expression following induced alterations of cellular shape. The international journal of biochemistry & cell biology 36, 1447-1461, doi:10.1016/j.biocel.2003.11.015 (2004).
Chen, C. S., Mrksich, M., Huang, S., Whitesides, G. M. & Ingber, D. E. Geometric control of cell life and death. Science (New York, N.Y.) 276, 1425-1428 (1997).
Schafer, Z. T. et al. Antioxidant and oncogene rescue of metabolic defects caused by loss of matrix attachment. Nature 461, 109-113, doi:10.1038/nature08268 (2009).
Weaver, V. M. et al. Reversion of the malignant phenotype of human breast cells in three-dimensional culture and in vivo by integrin blocking antibodies. The Journal of cell biology 137, 231-245 (1997).
Kang, Y. & Pantel, K. Tumor cell dissemination: emerging biological insights from animal models and cancer patients. Cancer Cell 23, 573-581, doi:10.1016/j.ccr.2013.04.017 (2013).
Gaggioli, C. et al. Fibroblast-led collective invasion of carcinoma cells with differing roles for RhoGTPases in leading and following cells. Nat Cell Biol 9, 1392-1400, doi:10.1038/ncb1658 (2007).
Hou, J. M. et al. Clinical significance and molecular characteristics of circulating tumor cells and circulating tumor microemboli in patients with small-cell lung cancer. J Clin Oncol 30, 525-532, doi:10.1200/jco.2010.33.3716 (2012).
Braun, S. & Naume, B. Circulating and disseminated tumor cells. J Clin Oncol 23, 1623-1626, doi:10.1200/jco.2005.10.073 (2005).
Short, B. in The Journal of cell biology Vol. 201 965 (2013).
Lang, N. R. et al. Estimating the 3D pore size distribution of biopolymer networks from directionally biased data. Biophys J 105, 1967-1975, doi:10.1016/j.bpj.2013.09.038 (2013).
Wolf, K. et al. in The Journal of cell biology Vol. 201 1069-1084 (2013).
Banerjee, P., Lenz, D., Robinson, J. P., Rickus, J. L. & Bhunia, A. K. A novel and simple cell-based detection system with a collagen-encapsulated B-lymphocyte cell line as a biosensor for rapid detection of pathogens and toxins. Laboratory investigation; a journal of technical methods and pathology 88, 196-206, doi:10.1038/labinvest.3700703 (2008).
Wills, P. R., Georgalis, Y., Dijk, J. & Winzor, D. J. Measurement of thermodynamic nonideality arising from volume-exclusion interactions between proteins and polymers. Biophysical chemistry 57, 37-46 (1995).
Bhat, R. & Timasheff, S. N. Steric exclusion is the principal source of the preferential hydration of proteins in the presence of polyethylene glycols. Protein science: a publication of the Protein Society 1, 1133-1143, doi:10.1002/pro.5560010907 (1992).
Bezrukov, S. M., Vodyanoy, I. & Parsegian, V. A. Counting polymers moving through a single ion channel. Nature 370, 279-281, doi:10.1038/370279a0 (1994).
Parsegian, V. A., Rand, R. P. & Rau, D. C. Osmotic stress, crowding, preferential hydration, and binding: A comparison of perspectives. Proceedings of the National Academy of Sciences of the United States of America 97, 3987-3992 (2000).
Kas, J. S., Tina, H. & Josef. Semiflexible Biopolymers in Bundled Arrangements. Polymers 8, 274, doi:10.3390/polym8080274 (2016).
Ji, H. et al. PEG-mediated osmotic stress induces premature differentiation of the root apical meristem and outgrowth of lateral roots in wheat. Journal of experimental botany 65, 4863-4872, doi:10.1093/jxb/eru255 (2014).
Parnaud, G., Corpet, D. E. & Gamet-Payrastre, L. Cytostatic effect of polyethylene glycol on human colonic adenocarcinoma cells. International journal of cancer 92, 63-69, doi:10.1002/1097-0215 (200102) 9999:9999<: : aid-ijc1158>3.0.co; 2-8 (2001).
Nieskoski, M. D. et al. Separation of Solid Stress From Interstitial Fluid Pressure in Pancreas Cancer Correlates With Collagen Area Fraction. Journal of biomechanical engineering 139, doi:10.1115/1.4036392 (2017).
Abramczyk, H., Brozek-Pluska, B., Surmacki, J., Jablonska-Gajewicz, J. & Kordek, R. Raman ‘optical biopsy’ of human breast cancer. Progress in biophysics and molecular biology 108, 74-81, doi:10.1016/j.pbiomolbio.2011.10.004 (2012).
Conklin, M. W. et al. Aligned collagen is a prognostic signature for survival in human breast carcinoma. The American journal of pathology 178, 1221-1232, doi:10.1016/j.ajpath.2010.11.076 (2011).
Provenzano, P. P. et al. Collagen density promotes mammary tumor initiation and progression. BMC medicine 6, 11, doi:10.1186/1741-7015-6-11 (2008).
Jawerth, L. M., Münster, S., Vader, D. A., Fabry, B. & Weitz, D. A. A Blind Spot in Confocal Reflection Microscopy: The Dependence of Fiber Brightness on Fiber Orientation in Imaging Biopolymer Networks. Biophysical journal 98, L1-3, doi:10.1016/j.bpj.2009.09.065 (2010).
Bredfeldt, J. S. et al. Computational segmentation of collagen fibers from second-harmonic generation images of breast cancer. J Biomed Opt 19, 16007, doi:10.1117/1.jbo.19.1.016007 (2014).
Johnson, D. O., Jeffrey, R., Haiyan, G., Thomas, F. F. & Mark. Specific Hydraulic Conductivity of Corneal Stroma as Seen by Quick-Freeze/Deep-Etch. Journal of biomechanical engineering 123, 154-161, doi:10.1115/1.1351888 (2017).
Johnson, T. D., Lin, S. Y. & Christman, K. L. Tailoring material properties of a nanofibrous extracellular matrix derived hydrogel. Nanotechnology 22, 494015, doi:10.1088/0957-4484/22/49/494015 (2011).
Radmacher, M., Fritz, M. & Hansma, P. K. Imaging soft samples with the atomic force microscope: gelatin in water and propanol. Biophys J 69, 264-270, doi:10.1016/s0006-3495 (95) 79897-6 (1995).

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A method of promoting a migration phenotype of a cancer or tumor cell in vitro, the method comprising embedding a cancer or tumor cell in a three-dimensional (3D) type-I collagen matrix; culturing the matrix for a period sufficient to elicit cell migration.

22. The method of claim 1, wherein the cancer or tumor cell is isolated from a cancer patient.

23. The method of claim 1, wherein the cancer or tumor cell is embedded as a single cell in the 3D type-I collagen matrix, or wherein a plurality of the cancer or tumor cells are embedded in the 3D type-I collagen matrix.

24. The method of claim 1, wherein the 3D type-I collagen matrix is a high density collagen matrix.

25. The method of claim 1, wherein a pore size of the 3D type-I collagen matrix has a coefficient of variation of less than 100%.

26. The method of claim 1, wherein the 3D type-I collagen matrix comprises a collagen density of greater than 4 mg/ml.

27. The method of claim 1, wherein the 3D type-I collagen matrix comprises a median fiber length of less than or equal to about 9.5 μm.

28. The method of claim 1, wherein the 3D type-I collagen matrix comprises a median pore size of less than or equal to about 10 μm.

29. The method of claim 1, wherein the 3D type-I collagen matrix is a low density collagen matrix further comprises a crowding agent.

30. The method of claim 9, wherein the crowding agent is selected from polyethylene glycol (PEG), polyvinyl alcohol, dextran, or ficoll.

31. The method of claim 9, wherein the concentration of the crowding agent in the 3D type-I collagen matrix is between about 4 mg/ml and about 10 mg/ml.

32. The method of claim 1, wherein the 3D type-I collagen matrix has been prepared by a method comprising the step of mixing an inert crowding agent into a type-I collagen monomer solution prior to polymerization of the 3D type-I collagen matrix or wherein the culturing comprises mixing the cancer or tumor cell suspended in a culture medium and a reconstitution buffer with soluble type I collagen or wherein the soluble type I collagen is in acetic acid, or wherein the culturing further comprises allowing the matrix to polymerize at a temperature of at least 37 °C.

33. The method of claim 1, further comprising determining the migration capacity of the cancer or tumor cell following at least one division cycle or the migration capacity of the cell is tracked by imaging the tumor cell.

34. The method of claim 1, further comprising, following culturing of the matrix, determining a gene expression level of one or more genes in the cancer or tumor cell selected from one or more of COL5A1, FRMD6, TANC2, THBS1, PEAK1, ITGAV, DAAM1, RASEF, JAG1, LAMC2, ZNF532, SKIL, NAV1, ARHGAP32, SYNE1, GALNTIO, LHFPL2, ABL2, LTBP1, COL4A1, DPY19L1, LPCAT2, TBC1D2B, LAMB1, AMIG02, NREP, SNX30, TPM1, COL4A2, ARNTL, MRC2, TGFBI, TVP23C, BHLHE40, SMAD7, ABLFM3, ZNF224, PODXL, TAGLN, VHL, EPHB2, EDN1, GTF2IP4, HPS4, SIPA1L1, PID1, NLGN2, LTBP4, TRMT13, IGF2BP3, RBPJ, MKL1, ZMYM5, EFCAB1 1, WDR66, NKX3-1, HMOX1, TYR03, SDHAP1, FURIN, FAM43A, AGTRAP, KCTD11, ID2, FERMT1, MTND2P28, H2BFS, LFNG, HES1, E-cadherin, CD24, Vimentin and KIN, and wherein gene expression is determined by a method comprising one or more of in situ hybridization, northern blot, PCR, quantitative PCR, RNA-seq, and microarray analysis.

35. The method of claim 1, wherein the migration phenotype comprises elongated single-cell invasion or formation of interconnected matrix networks.

36. A method for identifying an anti-metastatic drug, comprising:
(a) performing the method of claim 1 in a plurality of sample wells;
(b) contacting the cultured cell with a drug; and
(c) assaying the culture cell for an identifying behavior selected from a reduction or inhibition of cell viability or a reduction in at one metastatic behavior selected from invasion distance, network formation, viability, proliferation or gene expression relative to an untreated control, wherein the presence of at least one identifying behavior identifies the drug as an anti-metastatic drug.

37. The method of claim 16 wherein the plurality sample wells comprises at least 48 wells, or wherein the metastatic behavior is measured by confocal microscopy image analysis, or
wherein the cultured cell is contacted with different concentrations of one or more drug, different combinations of three or more drugs, and/or different dose times for one or more drugs, or wherein the method further comprises determining a cell viability in a well plate based on an assay, wherein the assay measures cell viability in response to a drug treatment to a given well in the well plate.

38. A method of:
a) quantifying network structures or determining the length network structures formed by a cancer or tumor cell,
b) determining an invasion distance of a cancer or tumor cell,
c) determining the shape of a cancer or tumor cell,
d) determining the viability, proliferation or death of a cancer or tumor cell, or
e) determining the probability of cancer or tumor cell metastasis in a subject, comprising promoting a migration phenotype in a cancer or tumor cell in vitro by the method of claim 1, and
a) quantifying the number of network structures or determining the length of network structures formed by a cancer or tumor cell following the culturing of the matrix,
b) determining the invasion distance of the cancer or tumor cell following the culturing of the matrix,
c) determining the shape of the cancer or tumor cell by one or more of time lapse imaging microscopy and/or confocal reflection imaging,
d) determining the viability, proliferation or death of the cancer or tumor cell by one or more of time lapse imaging microscopy, confocal reflection imaging, or a live/dead cell assay, or
e) determining the invasion distance of the tumor cell, wherein the cancer or tumor cell is from an early stage I or stage II cancer or tumor.

39. The method of claim 18, wherein the quantity or length of network structures are determined by a method comprising time lapse imaging microscopy and/or confocal reflection imaging, or wherein the method further comprises following culture of the matrix, determining a gene expression level of one or more genes in the cancer or tumor cell selected from one or more of E-cadherin, CD24, Vimentin, and a vasculogenic-mimicry gene module.

40. A kit comprising a cancer or tumor cell cultured by the method of claim 1.
